(19) United States
(12) Patent Application Publication
(10) Pub. No.: US 2011/0257223 A1
(43) Pub. Date: Oct. 20, 2011

(54) Modulators of Cystic Fibrosis Transmembrane Conductance Regulator

(75) Inventors: Fredrick Van Goor, San Diego, CA (US); Hayley Binch, Encinitas, CA (US); Martyn Botfield, Concord, MA (US); Lev T.D. Fanning, San Marcos, CA (US); Peter D.J. Grootenhuis, San Diego, CA (US); Dennis Hurley, San Marcos, CA (US); Medhi Michel Djamel Numa, San Diego, CA (US); Urvi Sheth, San Diego, CA (US); Alina Silina, San Diego, CA (US); Xiaoqing Yang, San Diego, CA (US); Gregor Zlokarnik, La Jolla, CA (US)

(73) Assignee: Vertex Pharmaceuticals Incorporated, Cambridge, MA (US)

(21) Appl. No.: 13/092,838

(22) Filed: Apr. 22, 2011

Related U.S. Application Data
(63) Continuation-in-part of application No. PCT/US2009/061882, filed on Oct. 23, 2009.
(60) Provisional application No. 61/327,095, filed on Apr. 22, 2010.
(60) Provisional application No. 61/107,830, filed on Oct. 23, 2008.

(51) Int. Cl. A61K 31/4709; A61P 11/06; A61P 11/08; A61P 1/10; A61P 11/00; A61P 15/00; A61P 1/16; A61P 1/18; A61P 3/10; A61P 35/00; A61P 21/00; A61P 25/28; A61P 25/16; A61P 25/00; A61P 27/02; A61P 19/10; A61P 19/08; A61P 25/08; G01N 33/567

(52) U.S. Cl. 514/304; 435/7.21; 514/312

(57) ABSTRACT

The present invention relates to modulators of cystic fibrosis transmembrane conductance regulator (“CFTR”), compositions thereof, and methods therewith. The present invention also relates to pharmaceutical compositions comprising a compound of Formula I with one or both of a Compound of Formula II and/or a Compound of Formula III. Further, the present invention relates to methods of treating CFTR mediated diseases, particularly cystic fibrosis, using modulators of CFTR, and compositions and combinations thereof.

### CROSS-REFERENCE TO RELATED APPLICATIONS

The present application claims the benefit of priority to U.S. provisional application 61/327,095, filed on Apr. 22, 2010, and is a Continuation-In-Part of International Application Serial No. PCT/US2009/061882, filed Oct. 23, 2009 (claiming the benefit of priority under 35 U.S.C. §120 and 35 U.S.C. §365(c)), which claims the benefit of priority to U.S. Provisional Application Ser. No. 61/107,830, filed Oct. 23, 2008 and is entitled “MODULATORS OF CYSTIC FIBROSIS TRANSMEMBRANE CONDUCTANCE REGULATOR,” the entire contents of the priority documents are incorporated herein by reference.

### TECHNICAL FIELD OF THE INVENTION

The present invention relates to modulators of cystic fibrosis transmembrane conductance regulator (“CFTR”), compositions thereof, and methods therewith. The present invention also relates to pharmaceutical compositions comprising a compound of Formula I with one or both of a Compound of Formula II and/or a Compound of Formula III. Further, the present invention relates to methods of treating CFTR mediated diseases, particularly cystic fibrosis, using modulators of CFTR, and compositions and combinations thereof.

### BACKGROUND OF THE INVENTION

ATP cassette transporters are a family of membrane transporter proteins that regulate the transport of a wide variety of pharmacological agents, potentially toxic drugs, and xenobiotics, as well as anions. They are homologous membrane proteins that bind and use cellular adenosine triphosphate (ATP) for their specific activities. Some of these transporters were discovered as multidrug resistance proteins (like the MDR1-P glycoprotein, or the multidrug resistance protein, MRP1), defending malignant cancer cells against chemotherapeutic agents. To date, 48 such transporters have been identified and grouped into 7 families based on their sequence identity and function.

One member of the ATP cassette transporters family commonly associated with disease is the cAMP/ATP-mediated anion channel, CFTR. CFTR is expressed in a variety of cells types, including absorptive and secretory epithelia cells, where it regulates anion flux across the membrane, as well as the activity of other ion channels and proteins. In epithelial cells, normal functioning of CFTR is critical for the maintenance of electrolyte transport throughout the body, including respiratory and digestive tissue. CFTR is composed of approximately 1480 amino acids that encode a protein made up of a tandem repeat of transmembrane domains, each containing six transmembrane helices and a nucleotide binding domain. The two transmembrane domains are linked by a large, polar, regulatory (R)-domain with multiple phosphorylation sites that regulate channel activity and cellular trafficking.

The gene encoding CFTR has been identified and sequenced (See Gregory, R. J. et al. (1990) Nature 347:382-386; Rich, D. P. et al. (1990) Nature 347:358-362), Riordan, J. R. et al. (1989) Science 245:1066-1073). A defect in this gene causes mutations in CFTR resulting in cystic fibrosis (“CF”), the most common fatal genetic disease in humans. Cystic fibrosis affects approximately one in every 2,500 infants in the United States. Within the general United States population, up to 10 million people carry a single copy of the defective gene without apparent ill effects. In contrast, individuals with two copies of the CF associated gene suffer from the debilitating and fatal effects of CF, including chronic lung disease.

In patients with cystic fibrosis, mutations in CFTR endogenously expressed in respiratory epithelia lead to reduced apical anion secretion causing an imbalance in ion and fluid transport. The resulting decrease in anion transport contributes to enhanced mucus accumulation in the lung and the accompanying microbial infections that ultimately cause death in CF patients. In addition to respiratory disease, CF patients typically suffer from gastrointestinal problems and pancreatic insufficiency that, if left untreated, results in death. In addition, the majority of males with cystic fibrosis are infertile and fertility is decreased among females with cystic fibrosis. In contrast to the severe effects of two copies of the CF associated gene, individuals with a single copy of the CF associated gene exhibit increased resistance to cholera and to dehydration resulting from diarrhea—perhaps explaining the relatively high frequency of the CF gene within the population.

Sequence analysis of the CFTR gene of CF chromosomes has revealed a variety of disease causing mutations (Cutting, G. R. et al. (1990) Nature 346:366-369; Dean, M. et al. (1990) Cell 61:863:870; and Kerem, B-S. et al. (1989) Science 245:1073-1080; Kerem, B-S et al. (1990) Proc. Natl. Acad. Sci. USA 87:8447-8451). To date, more than 1000 disease causing mutations in the CF gene have been identified (http://www.genet.sickkids.on.ca/cftr/). The most prevalent mutation is a deletion of phenylalanine at position 508 of the CFTR amino acid sequence, and is commonly referred to as ΔF508-CFTR. This mutation occurs in approximately 70 percent of the cases of cystic fibrosis and is associated with a severe disease.

The deletion of residue 508 in ΔF508-CFTR prevents the nascent protein from folding correctly. This results in the inability of the mutant protein to exit the ER, and traffic to the plasma membrane. As a result, the number of channels present in the membrane is far less than observed in cells expressing wild-type CFTR. In addition to impaired trafficking, the mutation results in defective channel gating. Together, the reduced number of channels in the membrane and the defective gating lead to reduced anion transport across epithelia, leading to defective ion and fluid transport. (Quinton, P. M. (1990), FASEB J. 4: 2709-2727). Studies have shown, however, that the reduced numbers of ΔF508-CFTR in the membrane are functional, albeit less than wild-type CFTR. (Dolmans et al. (1991), Nature Lond. 354: 526-528; Denning et al., supra; Pasyk and Foskett (1995), J. Cell. Biochem. 270: 12347-50). In addition to ΔF508-CFTR, R117H-CFTR and G551D-CFTR, other disease causing mutations in CFTR that result in defective trafficking, synthesis, and/or channel gating, could be up- or down-regulated to alter anion secretion and modify disease progression and/or severity.

Although CFTR transports a variety of molecules in addition to anions, it is clear that this role (the transport of anions, chloride and bicarbonate) represents one element in an important mechanism of transporting ions and water across the epithelium. The other elements include the epithelial Na⁺ channel, ENaC, Na⁺/2Cl⁻/K⁺ co-transporter, Na⁺—K⁺-ATPase pump and the basolateral membrane K⁺ channels, that are responsible for the uptake of chloride into the cell.

These elements work together to achieve directional transport across the epithelium via their selective expression and localization within the cell. Chloride absorption takes place by the coordinated activity of ENaC and CFTR present on the apical membrane and the Na⁺—K⁺-ATPase pump and Cl channels expressed on the basolateral surface of the cell. Secondary active transport of chloride from the luminal side leads to the accumulation of intracellular chloride, which can then passively leave the cell via Cl⁻ ion channels, resulting in a vectorial transport.

Arrangement of Na⁺/2Cl⁻/K⁺ co-transporter, Na⁺—K⁺-ATPase pump and the basolateral membrane IC channels on the basolateral surface and CFTR on the luminal side coordinate the secretion of chloride via CFTR on the luminal side. Because water is probably never actively transported itself, its flow across epithelia depends on tiny transepithelial osmotic gradients generated by the bulk flow of sodium and chloride.

Defective bicarbonate transport due to mutations in CFTR is hypothesized to cause defects in certain secretory functions. See, e.g., “Cystic fibrosis: impaired bicarbonate secretion and mucoviscidosis,” Paul M. Quinton, Lancet 2008; 372: 415-417.

Mutations in CFTR that are associated with moderate CFTR dysfunction are also evident in patients with conditions that share certain disease manifestations with CF but do not meet the diagnostic criteria for CF. These include congenital bilateral absence of the vas deferens, idiopathic chronic pancreatitis, chronic bronchitis, and chronic rhinosinusitis. Other diseases in which mutant CFTR is believed to be a risk factor along with modifier genes or environmental factors include primary sclerosing cholangitis, allergic bronchopulmonary aspergillosis, and asthma.

Cigarette smoke, hypoxia, and environmental factors that induce hypoxic signaling have also been demonstrated to impair CFTR function and may contribute to certain forms of respiratory disease, such as chronic bronchitis. Diseases that may be due to defective CFTR function but do not meet the diagnostic criteria for CF are characterized as CFTR-related diseases.

In addition to cystic fibrosis, modulation of CFTR activity may be beneficial for other diseases not directly caused by mutations in CFTR, such as secretory diseases and other protein folding diseases mediated by CFTR. CFTR regulates chloride and bicarbonate flux across the epithelia of many cells to control fluid movement, protein solubilization, mucus viscosity, and enzyme activity. Defects in CFTR can cause blockage of the airway or ducts in many organs, including the liver and pancreas. Potentiators are compounds that enhance the gating activity of CFTR present in the cell membrane. Any disease which involves thickening of the mucus, impaired fluid regulation, impaired mucus clearance, or blocked ducts leading to inflammation and tissue destruction could be a candidate for potentiators. Another potential therapeutic strategy involves small molecule drugs known as CF correctors that increase the number and function of CFTR channels.

These include, but are not limited to, chronic obstructive pulmonary disease (COPD), asthma, smoke induced COPD, chronic bronchitis, rhinosinusitis, constipation, dry eye disease, and Sjögren's Syndrome, gastroesophageal reflux disease, gallstones, rectal prolapse, and inflammatory bowel disease. COPD is characterized by airflow limitation that is progressive and not fully reversible. The airflow limitation is due to mucus hypersecretion, emphysema, and bronchiolitis. Activators of mutant or wild-type CFTR offer a potential treatment of mucus hypersecretion and impaired mucociliary clearance that is common in COPD. Specifically, increasing anion secretion across CFTR may facilitate fluid transport into the airway surface liquid to hydrate the mucus and optimized periciliary fluid viscosity. This would lead to enhanced mucociliary clearance and a reduction in the symptoms associated with COPD. In addition, by preventing ongoing infection and inflammation due to improved airway clearance, CFTR modulators may prevent or slow the parenchimal destruction of the airway that characterizes emphysema and reduce or reverse the increase in mucus secreting cell number and size that underlyses mucus hypersecretion in airway diseases. Dry eye disease is characterized by a decrease in tear aqueous production and abnormal tear film lipid, protein and mucin profiles. There are many causes of dry eye, some of which include age, Lasik eye surgery, arthritis, medications, chemical/thermal burns, allergies, and diseases, such as cystic fibrosis and Sjögren's syndrome. Increasing anion secretion via CFTR would enhance fluid transport from the corneal endothelial cells and secretory glands surrounding the eye to increase corneal hydration. This would help to alleviate the symptoms associated with dry eye disease. Sjögrens's syndrome is an autoimmune disease in which the immune system attacks moisture-producing glands throughout the body, including the eye, mouth, skin, respiratory tissue, liver, vagina, and gut. Symptoms, include, dry eye, mouth, and vagina, as well as lung disease. The disease is also associated with rheumatoid arthritis, systemic lupus, systemic sclerosis, and polymypositis/dermatomyositis. Defective protein trafficking is believed to cause the disease, for which treatment options are limited. Modulators of CFTR activity may hydrate the various organs afflicted by the disease and may help to alleviate the associated symptoms. Individuals with cystic fibrosis have recurrent episodes of intestinal obstruction and higher incidences of rectal polapse, gallstones, gastroesophageal reflux disease, GI malignancies, and inflammatory bowel disease, indicating that CFTR function may play an important role in preventing such diseases.

As discussed above, it is believed that the deletion of residue 508 in ΔF508-CFTR prevents the nascent protein from folding correctly, resulting in the inability of this mutant protein to exit the ER, and traffic to the plasma membrane. As a result, insufficient amounts of the mature protein are present at the plasma membrane and chloride transport within epithelial tissues is significantly reduced. In fact, this cellular phenomenon of defective ER processing of CFTR by the ER machinery, has been shown to be the underlying basis not only for CF disease, but for a wide range of other isolated and inherited diseases. The two ways that the ER machinery can malfunction is either by loss of coupling to ER export of the proteins leading to degradation, or by the ER accumulation of these defective/misfolded proteins [Aridor M, et al., Nature Med., 5(7), pp 745-751 (1999); Shastry, B. S., et al., Neurochem. International, 43, pp 1-7 (2003); Rutishauser, J., et al., Swiss Med Wkly, 132, pp 211-222 (2002); Morello, J P et al., TIPS, 21, pp. 466-469 (2000); Bross P., et al., Human Mut., 14, pp. 186-198 (1999)]. The diseases associated with the first class of ER malfunction are cystic fibrosis (due to misfolded ΔF508-CFTR as discussed above), hereditary emphysema (due to a1-antitrypsin; non Piz variants), hereditary hemochromatosis, coagulation-fibrinolysis deficiencies, such as protein C deficiency, Type 1 hereditary angioedema, lipid processing deficiencies, such as familial hypercholesterolemia, Type 1 chylomicronemia, abetalipoproteinemia, lysosomal storage diseases, such as I-cell disease/pseudo-Hurler, Mucopolysaccharidoses (due to lysosomal processing enzymes), Sandhof/Tay-Sachs (due to β-hexosaminidase), Crigler-Najjar type II (due to UDP-glucuronyl-sialyc-transferase), polyendocrinopathy/hyperinsulemia, Diabetes mellitus (due to insulin receptor), Laron dwarfism (due to growth hormone receptor), myleoperoxidase deficiency, primary hypoparathyroidism (due to preproparathyroid hormone), melanoma (due to tyrosinase). The diseases associated with the latter class of ER malfunction are Glycanosis CDG type 1, hereditary emphysema (due to al-Antitrypsin (PiZ variant), congenital hyperthyroidism, osteogenesis imperfecta (due to Type I, II, IV procollagen), hereditary hypofibrinogenemia (due to fibrinogen), ACT deficiency (due to α1-antichymotrypsin), Diabetes insipidus (DI), neurophyseal DI (due to vasopvessin hormone/V2-receptor), neprogenic DI (due to aquaporin II), Charcot-Marie Tooth syndrome (due to peripheral myelin protein 22), Perlizaeus-Merzbacher disease, neurodegenerative diseases such as Alzheimer's disease (due to βAPP and presenilins), Parkinson's disease, amyotrophic lateral sclerosis, progressive supranuclear palsy, Pick's disease, several polyglutamine neurological disorders such as Huntington's, spinocerebullar ataxia type I, spinal and bulbar muscular atrophy, dentatorubal pallidoluysian, and myotonic dystrophy, as well as spongiform encephalopathies, such as hereditary Creutzfeldt-Jakob disease (due to prion protein processing defect), Fabry disease (due to lysosomal α-galactosidase A), Straussler-Scheinker syndrome (due to Prp processing defect), infertility pancreatitis, pancreatic insufficiency, osteoporosis, osteopenia, Gorham's Syndrome, chloride channelopathies, myotonia congenita (Thomson and Becker forms), Bartter's syndrome type III, Dent's disease, epilepsy, hyperekplexia, lysosomal storage disease, Angelman syndrome, Primary Ciliary Dyskinesia (PCD), PCD with situs inversus (also known as Kartagener syndrome), PCD without situs inversus and ciliary aplasia, and liver disease.

Other diseases implicated by a mutation in CFTR include male infertility caused by congenital bilateral absence of the vas deferens (CBAVD), mild pulmonary disease, idiopathic pancreatitis, and allergic bronchopulmonary aspergillosis (ABPA). See, “CFTR-opathies: disease phenotypes associated with cystic fibrosis transmembrane regulator gene mutations,” Peader G. Noone and Michael R. Knowles, Respir. Res. 2001, 2: 328-332 (incorporated herein by reference).

In addition to up-regulation of CFTR activity, reducing anion secretion by CFTR modulators may be beneficial for the treatment of secretory diarrheas, in which epithelial water transport is dramatically increased as a result of secretagogue activated chloride transport. The mechanism involves elevation of cAMP and stimulation of CFTR.

Although there are numerous causes of diarrhea, the major consequences of diarrheal diseases, resulting from excessive chloride transport are common to all, and include dehydration, acidosis, impaired growth and death. Acute and chronic diarrheas represent a major medical problem in many areas of the world. Diarrhea is both a significant factor in malnutrition and the leading cause of death (5,000,000 deaths/year) in children less than five years old.

Secretory diarrheas are also a dangerous condition in patients with acquired immunodeficiency syndrome (AIDS) and chronic inflammatory bowel disease (IBD). Sixteen million travelers to developing countries from industrialized nations every year develop diarrhea, with the severity and number of cases of diarrhea varying depending on the country and area of travel.

Accordingly, there is a need for potent and selective CFTR potentiators of wild-type and mutant forms of human CFTR. These mutant CFTR forms include, but are not limited to, ΔF508del, G551D, R117H, 2789+5G->A.

Compounds which are potentiators of CFTR protein, such as those of Formula I, and compounds which are correctors of CFTR protein, such as those of Formula II or Formula III, have been shown independently to have utility in the treatment of CFTR modulated diseases, such as Cystic Fibrosis.

Accordingly, there is a need for novel treatments of CFTR mediated diseases which involve CFTR corrector and potentiator compounds.

Particularly, there is a need for combination therapies to treat CFTR mediated diseases, such as Cystic Fibrosis, which include CFTR potentiator and corrector compounds.

More particularly, there is a need for combination therapies to treat CFTR mediated diseases, such as Cystic Fibrosis, which include CFTR potentiator compounds, such as compounds of Formula I, in combination with CFTR corrector compounds such as compounds of Formula II and/or Formula III.

Even more particularly, there is a need for therapies to treat CFTR mediated diseases, such as Cystic Fibrosis, comprising a CFTR potentiator compound such as Compound 1, in combination with a CFTR corrector such as Compound 2 and/or Compound 3.

There is also a need for modulators of CFTR activity, and compositions thereof, which can be used to modulate the activity of the CFTR in the cell membrane of a mammal.

There is a need for methods of treating diseases caused by mutation in CFTR using such modulators of CFTR activity.

There is a need for methods of modulating CFTR activity in an ex vivo cell membrane of a mammal.

### SUMMARY OF THE INVENTION

It has now been found that compounds of this invention, and pharmaceutically acceptable compositions thereof, are useful as modulators of CFTR activity. The compounds have the general Formula I:

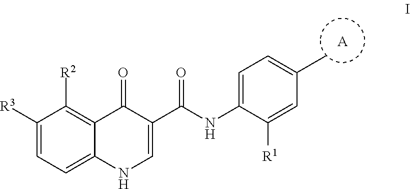

or pharmaceutically acceptable salts thereof, wherein R¹, R², R³ and A are described generally and in classes and subclasses below.

In another aspect, the invention is directed to a pharmaceutical composition comprising a first component, which is selected from Column A of Table I, and a second component, which is selected from one or both of Column B and/or Column C of Table I. These components are described in the corresponding sections of the following pages as embodiments of the invention. For convenience, Table I recites the section number and corresponding heading title of the embodiments of the compounds, solid forms and formulations.

TABLE I

| Column A: | | Column B: | | Column C: | |
|---|---|---|---|---|---|
| Potentiator Component | | Corrector Component | | Corrector Component | |
| Embodiments | | Embodiments | | Embodiments | |
| Section | Heading | Section | Heading | Section | Heading |
| II.A.1 | Compounds of | II.B.1 | Compounds of | II.C.1 | Compounds of |
| | Formula I | | Formula II | | Formula III |
| II.A.2 | Compound 1 | II.B.2 | Compound 2 | II.C.2 | Compound 3 |

These compounds, combinations and pharmaceutically acceptable compositions are useful for treating or lessening the severity of a variety of diseases, disorders, or conditions associated with mutations in CFTR.

In one aspect, the invention includes a pharmaceutical composition comprising a component selected from any embodiment described in Column A of Table I in combination with a component selected from any embodiment described in Column B and/or a component selected from any embodiment described in Column C of Table I.

Thus, in one embodiment, the invention is directed to a pharmaceutical composition comprising a Compound of Formula I and a Compound of Formula II.

In another embodiment, the invention is directed to a pharmaceutical composition comprising a Compound of Formula I and a Compound of Formula III.

In a further embodiment, the invention is directed to a pharmaceutical composition comprising a Compound of Formula I, a Compound of Formula II and a Compound of Formula III.

In another embodiment, the invention is directed to a pharmaceutical composition comprising Compound 1 and a Compound of Formula II.

In another embodiment, the invention is directed to a pharmaceutical composition comprising Compound 1 and a Compound of Formula III.

In another embodiment, the invention is directed to a pharmaceutical composition comprising Compound 1, a Compound of Formula II and a Compound of Formula III.

In another embodiment, the invention is directed to a pharmaceutical composition comprising Compound 1 and Compound 2.

In another embodiment, the invention is directed to a pharmaceutical composition comprising Compound 1 and Compound 3.

In another embodiment, the invention is directed to a pharmaceutical composition comprising Compound 1, Compound 2 and Compound 3.

In another embodiment, the invention is directed to a pharmaceutical composition comprising a Compound of Formula I and Compound 2.

In another further embodiment, the invention is directed to a pharmaceutical composition comprising a Compound of Formula I and Compound 3.

In another further embodiment, the invention is directed to a pharmaceutical composition comprising a Compound of Formula I, Compound 2 and Compound 3.

Various components listed in Table I have been disclosed and can be found in U.S. Pat. No. 7,776,905, U.S. Pat. No. 7,645,789, US 2010/0113508, US 2010/0130547, US 2008/0113985A1, US2008/0019915A1, US 2008/0306062A1, US 2009/0170905 A1, US 2009/0176839 and US 2010/0087490 the contents of which are incorporated herein by reference.

### DETAILED DESCRIPTION OF THE INVENTION

### I. Compounds and Definitions

Compounds of this invention include those described generally above, and are further illustrated by the classes, subclasses, and species disclosed herein. As used herein, the following definitions shall apply unless otherwise indicated.

The term “ABC-transporter” as used herein means an ABC-transporter protein or a fragment thereof comprising at least one binding domain, wherein said protein or fragment thereof is present in vivo or in vitro. The term “binding domain” as used herein means a domain on the ABC-transporter that can bind to a modulator. See, e.g., Hwang, T. C. et al., J. Gen. Physiol. (1998): 111(3), 477-90.

The term “CFTR” as used herein means cystic fibrosis transmembrane conductance regulator or a mutation thereof capable of regulator activity, including, but not limited to, ΔF508 CFTR, R117H CFTR, and G551D CFTR (see, e.g., http://www.genet.sickkids.on.ca/cftr/, for CFTR mutations).

The term “modulating” as used herein means increasing or decreasing by a measurable amount.

The term “normal CFTR” or “normal CFTR function” as used herein means wild-type like CFTR without any impairment due to environmental factors such as smoking, pollution, or anything that produces inflammation in the lungs.

The term “reduced CFTR” or “reduced CFTR function” as used herein means less than normal CFTR or less than normal CFTR function.

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-20 aliphatic carbon atoms. In some embodiments, aliphatic groups contain 1-10 aliphatic carbon atoms. In other embodiments, aliphatic groups contain 1-8 aliphatic carbon atoms. In still other embodiments, aliphatic groups contain 1-6 aliphatic carbon atoms, and in yet other embodiments aliphatic groups contain 1-4 aliphatic carbon atoms. In some embodiments, “cycloaliphatic” (or “carbocycle” or “cycloalkyl”) refers to a monocyclic C₃-C₈ hydrocarbon or bicyclic or tricyclic C₈-C₁₄ hydrocarbon that is completely saturated or that contains one or more units of unsaturation, but which is not aromatic, that has a single point of attachment to the rest of the molecule wherein any individual ring in said bicyclic ring system has 3-7 members. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl. Suitable cycloaliphatic groups include cycloalkyl, bicyclic cycloalkyl (e.g., decalin), bridged bicycloalkyl such as norbornyl or [2.2.2]bicyclo-octyl, or bridged tricyclic such as adamantyl.

The term “alkyl” as used herein refers to a saturated aliphatic hydrocarbon group containing 1-15 (including, but not limited to, 1-8, 1-6, 1-4, 2-6, 3-12) carbon atoms. An alkyl group can be straight or branched.

The term “heteroaliphatic,” as used herein, means aliphatic groups wherein one or two carbon atoms are independently replaced by one or more of oxygen, sulfur, nitrogen, phosphorus, or silicon. Heteroaliphatic groups may be substituted or unsubstituted, branched or unbranched, cyclic or acyclic, and include “heterocycle,” “heterocyclyl,” “heterocycloaliphatic,” or “heterocyclic” groups.

The term “heterocycle,” “heterocyclyl,” “heterocycloaliphatic,” or “heterocyclic” as used herein means non-aromatic, monocyclic, bicyclic, or tricyclic ring systems in which one or more ring members is an independently selected heteroatom. In some embodiments, the “heterocycle,” “heterocyclyl,” “heterocycloaliphatic,” or “heterocyclic” group has three to fourteen ring members in which one or more ring members is a heteroatom independently selected from oxygen, sulfur, nitrogen, or phosphorus, and each ring in the system contains 3 to 7 ring members.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

The term “aryl” used alone or as part of a larger moiety as in “aralkyl,” “aralkoxy,” or “aryloxyalkyl,” refers to monocyclic, bicyclic, and tricyclic ring systems having a total of five to fourteen ring members, wherein at least one ring in the system is aromatic and wherein each ring in the system contains 3 to 7 ring members. The term “aryl” may be used interchangeably with the term “aryl ring.” The term “aryl” also refers to heteroaryl ring systems as defined herein below.

An aliphatic or heteroaliphatic group, or a non-aromatic heterocyclic ring may contain one or more substituents. Suitable substituents on the saturated carbon of an aliphatic or heteroaliphatic group, or of a non-aromatic heterocyclic ring are selected from those listed above for the unsaturated carbon of an aryl or heteroaryl group and additionally include the following: ═O, ═S, ═NNHR*, ═NN(R*)₂, ═NNHC(O)R*, ═NNHCO₂(alkyl), ═NNHSO₂(alkyl), or ═NR*, where each R* is independently selected from hydrogen or an optionally substituted C₁₋₆ aliphatic. Optional substituents on the aliphatic group of R* are selected from NH₂, NH(C₁₋₄ aliphatic), N(C₁₋₄ aliphatic)₂, halo, C₁₋₄ aliphatic, OH, O(C₁₋₄ aliphatic), NO₂, CN, CO₂H, CO₂(C₁₋₄ aliphatic), O(halo C₁₋₄ aliphatic), or halo(C₁₋₁₄ aliphatic), wherein each of the foregoing C₁₋₄aliphatic groups of R* is unsubstituted.

Optional substituents on the nitrogen of a non-aromatic heterocyclic ring are selected from —R⁺, —N(R⁺)₂, —C(O)R⁺, —CO₂R⁺, —C(O)C(O)R⁺, —C(O)CH₂C(O)R⁺, —SO₂R⁺, —SO₂N(R⁺)₂, —C(═S)N(R⁺)₂, —C(═NH)—N(R⁺)₂, or —NR⁺SO₂R⁺; wherein R⁺ is hydrogen, an optionally substituted C₁₋₆ aliphatic, optionally substituted phenyl, optionally substituted —O(Ph), optionally substituted —CH₂(Ph), optionally substituted —(CH₂)₁₋₂(Ph); optionally substituted —CH═CH(Ph); or an unsubstituted 5-6 membered heteroaryl or heterocyclic ring having one to four heteroatoms independently selected from oxygen, nitrogen, or sulfur, or, notwithstanding the definition above, two independent occurrences of R⁺, on the same substituent or different substituents, taken together with the atom(s) to which each R⁺ group is bound, form a 3-8-membered cycloalkyl, heterocyclyl, aryl, or heteroaryl ring having 0-3 heteroatoms independently selected from nitrogen, oxygen, or sulfur. Optional substituents on the aliphatic group or the phenyl ring of R⁺ are selected from NH₂, NH(C₁₋₄ aliphatic), N(C₁₋₁₄ aliphatic)₂, halo, C₁₋₄ aliphatic, OH, O(C₁₋₄ aliphatic), NO₂, CN, CO₂H, CO₂(C₁₋₄ aliphatic), O(halo C₁₋₄ aliphatic), or halo(C₁₋₁₄ aliphatic), wherein each of the foregoing C₁₋₄aliphatic groups of R⁺ is unsubstituted.

As detailed above, in some embodiments, two independent occurrences of R′ (or any other variable similarly defined herein), are taken together with the atom(s) to which each variable is bound to form a 3-8-membered cycloalkyl, heterocyclyl, aryl, or heteroaryl ring having 0-3 heteroatoms independently selected from nitrogen, oxygen, or sulfur. Exemplary rings that are formed when two independent occurrences of R′ (or any other variable similarly defined herein) are taken together with the atom(s) to which each variable is bound include, but are not limited to the following: a) two independent occurrences of R′ (or any other variable similarly defined herein) that are bound to the same atom and are taken together with that atom to form a ring, for example, N(R′)₂, where both occurrences of R′ are taken together with the nitrogen atom to form a piperidin-1-yl, piperazin-1-yl, or morpholin-4-yl group; and b) two independent occurrences of R′ (or any other variable similarly defined herein) that are bound to different atoms and are taken together with both of those atoms to form a ring, for example where a phenyl group is substituted with two occurrences of OR′

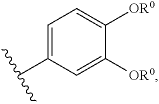

these two occurrences of R^{o} are taken together with the oxygen atoms to which they are bound to form a fused 6-membered oxygen containing ring:

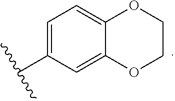

It will be appreciated that a variety of other rings can be formed when two independent occurrences of R′ (or any other variable similarly defined herein) are taken together with the atom(s) to which each variable is bound and that the examples detailed above are not intended to be limiting.

A substituent bond in, e.g., a bicyclic ring system, as shown below, means that the substituent can be attached to any substitutable ring atom on either ring of the bicyclic ring system:

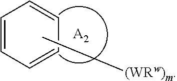

For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

Combinations of substituents envisioned by this invention are preferably those that result in the formation of stable or chemically feasible compounds. The term “stable”, as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

The term “protecting group,” as used herein, refers to an agent used to temporarily to block one or more desired reactive sites in a multifunctional compound. In certain embodiments, a protecting group has one or more, or preferably all, of the following characteristics: a) reacts selectively in good yield to give a protected substrate that is stable to the reactions occurring at one or more of the other reactive sites; and b) is selectively removable in good yield by reagents that do not attack the regenerated functional group. Exemplary protecting groups are detailed in Greene, T. W., Wuts, P. G in “Protective Groups in Organic Synthesis”, Third Edition, John Wiley & Sons, New York: 1999, and other editions of this book, the entire contents of which are hereby incorporated by reference.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention; e.g., compounds of Formula I may exist as tautomers:

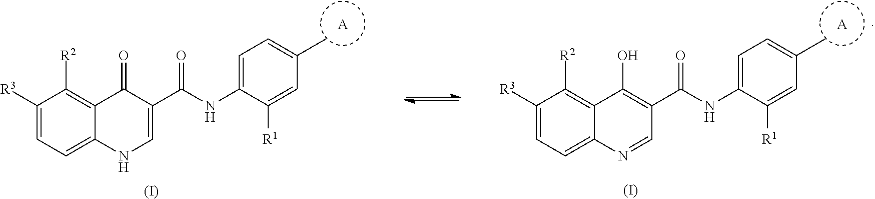

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C— or ¹⁴C-enriched carbon are within the scope of this invention. Such compounds are useful, for example, as analytical tools or probes in biological assays. Such compounds, particularly compounds that contain deuterium atoms, may exhibit modified metabolic properties.

### II. Compounds of the Invention

In one aspect, the invention is directed to a compound of Formula I

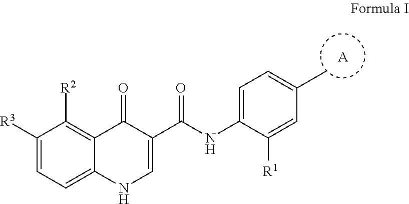

In another aspect, the invention is directed to a pharmaceutical composition comprising a compound of Formula I in combination with a Compound of Formula II and/or a Compound of Formula III.

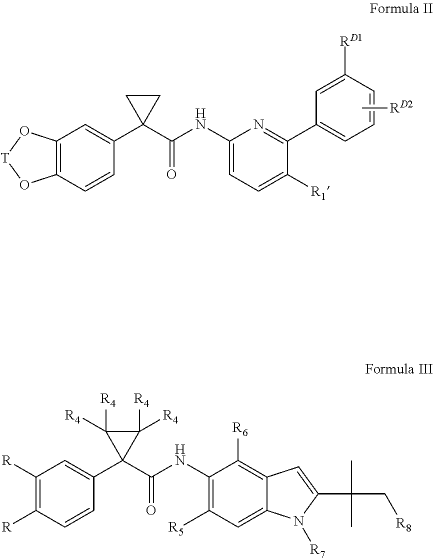

### II.A. Compounds of Formula I

### II.A.1. Embodiments of the Compounds of Formula I

In one aspect, the present invention relates to compounds of Formula I, and pharmaceutical compositions comprising compounds of Formula I, which are useful as modulators of CFTR activity:

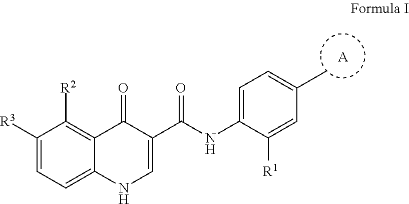

or pharmaceutically acceptable salts thereof, wherein:
ring A is selected from:

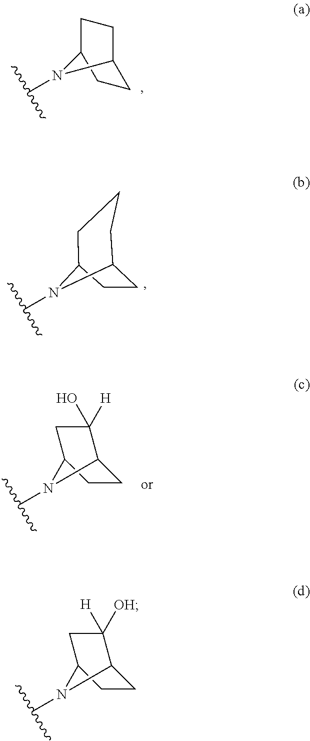

R¹ is —CF₃, —CN, or —C≡CH₂N(CH₃)₂;
R² is hydrogen, —CH₃, —CF₃, —OH, or —CH₂OH;
R³ is hydrogen, —CH₃, —OCH₃, or —CN;

provided that both R² and R³ are not simultaneously hydrogen;

In one embodiment, ring A of Formula I is

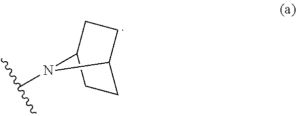

In one embodiment, ring A of Formula I is

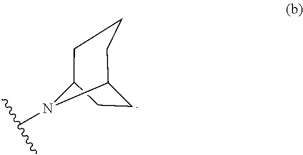

In another embodiment, ring A of Formula I is

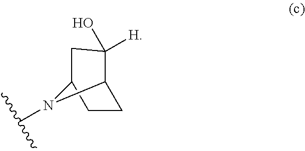

In yet another embodiment, ring A of Formula I is

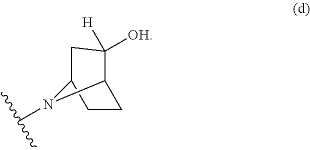

In one embodiment, R¹ of Formula I is —CF₃.

In another embodiment, R¹ of Formula I is —CN.

In another embodiment, R¹ of Formula I is —C≡CCH₂N(CH₃)₂.

In one embodiment, R² of Formula I is —CH₃.

In another embodiment, R² of Formula I is —CF₃.

In another embodiment, R² of Formula I is —OH.

In another embodiment, R² of Formula I is —CH₂OH.

In one embodiment, R³ of Formula I is —CH₃.

In one embodiment, R³ of Formula I is —OCH₃.

In another embodiment, R³ of Formula I is —CN.

In one embodiment, R² of Formula I is hydrogen; and R³ of Formula I is —CH₃, —OCH₃, or —CN.

In another embodiment, R² of Formula I is —CH₃, —CF₃, —OH, or —CH₂OH; and R³ of Formula I is hydrogen.

In several embodiments of the present invention, ring A of Formula I is

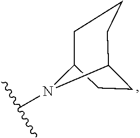

is —CF₃, R² is hydrogen; and R³ is —CH₃, —OCH₃, or —CN. In other embodiments, R¹ is —CN. In still further embodiments, R¹ is —C≡CCH₂N(CH₃)₂. In one embodiment, R³ is —CH₃. Or, R³ is —OCH₃. Or, R³ is —CN.

In further embodiments of the present invention, ring A of Formula I

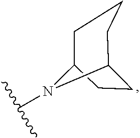

is R¹ is —CF₃, R² is —CH₃, —CF₃, —OH, or —CH₂OH, and R³ is hydrogen. In other embodiments, R¹ is —CN. In still further embodiments, R¹ is —C≡CCH₂N(CH₃)₂. In one embodiment, R² is —CH₃. Or, R² is —CF₃. Or, R² is —OH. Or, R² is —CH₂OH.

In several embodiments of the present invention, ring A of Formula I

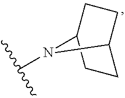

is is —CF₃, R² is hydrogen; and R³ is —CH₃, —OCH₃, or —CN. In other embodiments, R¹ is —CN. In still further embodiments, R¹ is —C≡CCH₂N(CH₃)₂. In one embodiment, R³ is —OCH₃. Or, R³ is —CH₃. Or, R³ is —CN.

In further embodiments of the present invention, ring A of Formula I

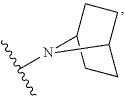

is is —CF₃, R² is —CH₃, —CF₃, —OH, or —CH₂OH, and R³ is hydrogen. In other embodiments, R¹ is —CN. In still further embodiments, R¹ is —C≡CCH₂N(CH₃)₂. In one embodiment, R² is —CH₃. Or, R² is —CF₃. Or, R² is —OH. Or, R² is —CH₂OH.

In several embodiments of the present invention, ring A of Formula I

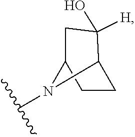

is R¹ is —CF₃, R² is hydrogen; and R³ is —CH₃, —OCH₃, or —CN. In other embodiments, R¹ is —CN. In still further embodiments, R¹ is —C≡CCH₂N(CH₃)₂. In one embodiment, R³ is —CH₃. Or, R³ is —OCH₃. Or, R³ is —CN.

In further embodiments of the present invention, ring A of Formula I

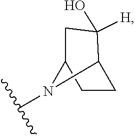

is R¹ is —CF₃, R² is —CH₃, —CF₃, —OH, or —CH₂OH, and R³ is hydrogen. In other embodiments, R¹ is —CN. In still further embodiments, R¹ is —C≡CCH₂N(CH₃)₂. In one embodiment, R² is —CH₃. Or, R² is —CF₃. Or, R² is —OH. Or, R² is —CH₂OH.

In several embodiments of the present invention, ring A of Formula I

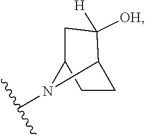

is R¹ is —CF₃, R² is hydrogen; and R³ is —CH₃, —OCH₃, or —CN. In other embodiments, R¹ is —CN. In still further embodiments, R¹ is —C≡CCH₂N(CH₃)₂. In one embodiment, R³ is —CH₃. Or, R³ is —OCH₃. Or, R³ is —CN.

In further embodiments of the present invention, ring A of Formula I

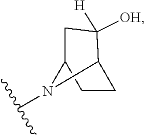

is R¹ is —CF₃, R² is —CH₃, —CF₃, —OH, or —CH₂OH, and R³ is hydrogen. In other embodiments, R¹ is —CN. In still further embodiments, R¹ is —C≡CCH₂N(CH₃)₂. In one embodiment, R² is —CH₃. Or, R² is —CF₃. Or, R² is —OH. Or, R² is —CH₂OH.

Representative compounds of Formula I are set forth in Table 1-1 below.

TABLE 1-1 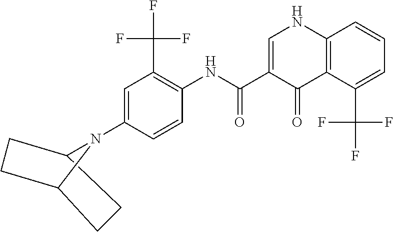
1 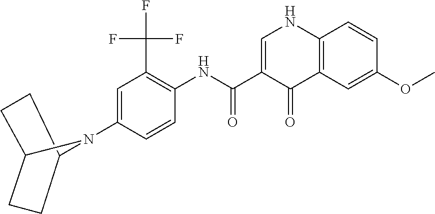
1-2 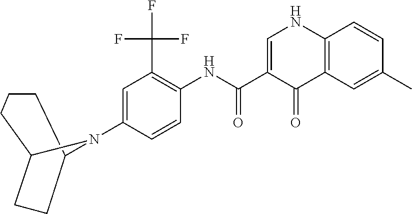
1-3 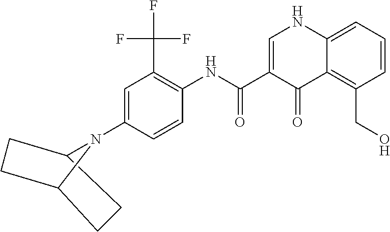
1-4 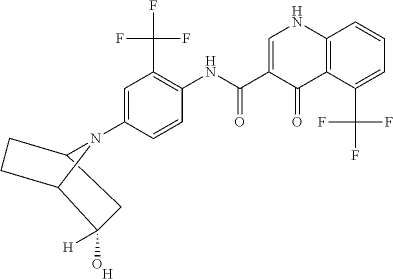
1-5 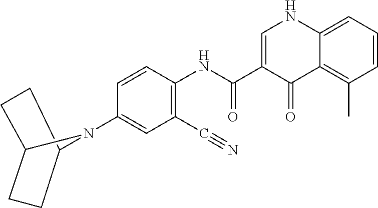
1-6 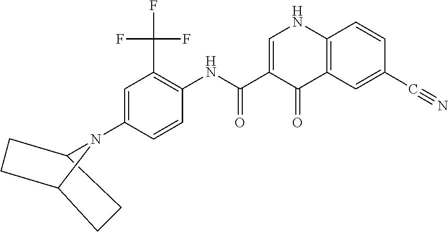
1-7 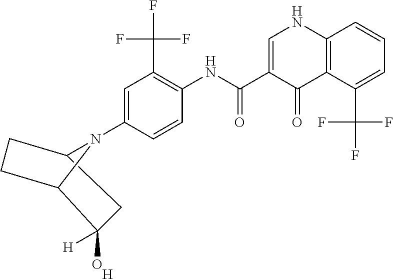
1-8 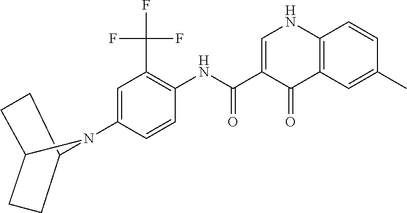
1-9 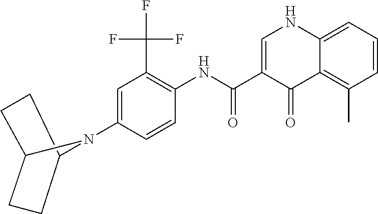
1-10 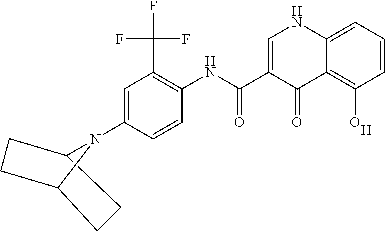
1-11 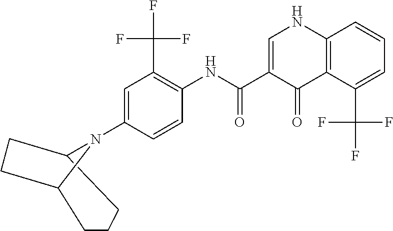
1-12 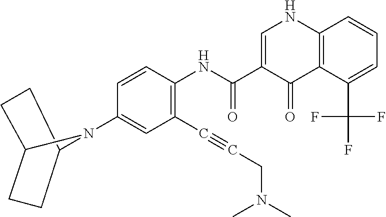
1-13 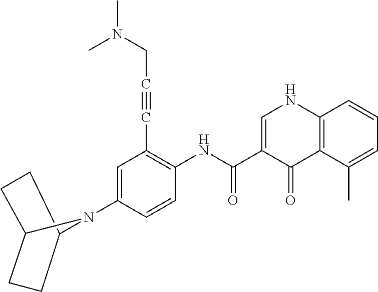
1-14

### II.A.2. Compound 1

In another embodiment, the Compound of Formula I is Compound 1, which is known by its chemical name N-(4-(7-azabicyclo[2.2.1]heptan-7-yl)-2-(trifluoromethyl)phenyl)-4-oxo-5-(trifluoromethyl)-1,4-dihydroquinoline-3-carboxamide.

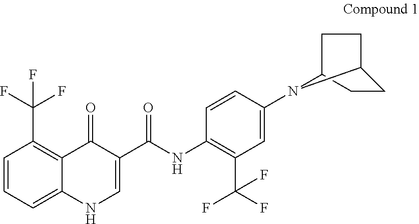

### II.A.3. Synthesis of Formula I Compounds

II.A.3.a. General Schemes

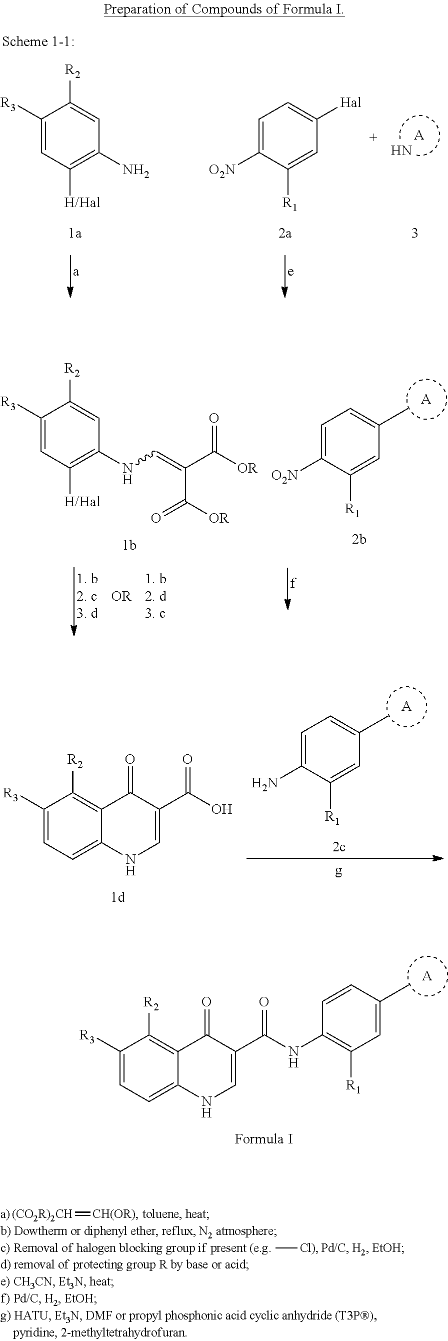

Scheme 1-1 depicts a convergent approach to the preparation of compounds of Formula I from substituted benzene derivatives 1a and 2a. In the ultimate transformation, amide formation via coupling of carboxylic acid 1d with amine 2c to give a compound of Formula I can be achieved using either O-(7-azabenzotriazol-1-yl)-N,N,N′,N′-tetramethyluronium hexafluorophosphate (HATU) and triethylamine in N,N-dimethyl formamide (DMF) or propyl sulfronic acid cyclic anhydride (T3P®) and pyridine in 2-methyltetrahydrofuran. Carboxylic acid 1d is prepared from the corresponding substituted benzene derivative 1a via a sequence commencing with heat-mediated condensation of 1a with an appropriate malonate (CO₂R)₂CH═CH(OR), wherein R is an alkyl or aryl group such as methyl, ethyl, t-butyl, phenyl, p-nitro phenyl or the like, to provide 1b.

Compound 1b is converted to carboxylic acid 1d via a three step sequence including intramolecular cyclization upon heating at reflux in Dowtherm or diphenyl ether (step b), followed by removal (if needed) of the blocking halo group (step c) under palladium-catalyzed dehalogenation conditions and acid- or base-catalyzed saponification (step d). The order of the deprotection and saponification steps can be reversed; i.e., step c can occur before or after step d, as depicted in Scheme 1-1.

Referring again to Scheme 1-1, aniline derivative 2c can be prepared from nitrobenzene 2a via a three step sequence. Thus, coupling of nitrobenzene 2a with a cyclic amine

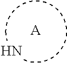

3 as defined herein in the presence of triethylamine provides compound 2b. Palladium-catalyzed reduction of 2b provides amine 2c.

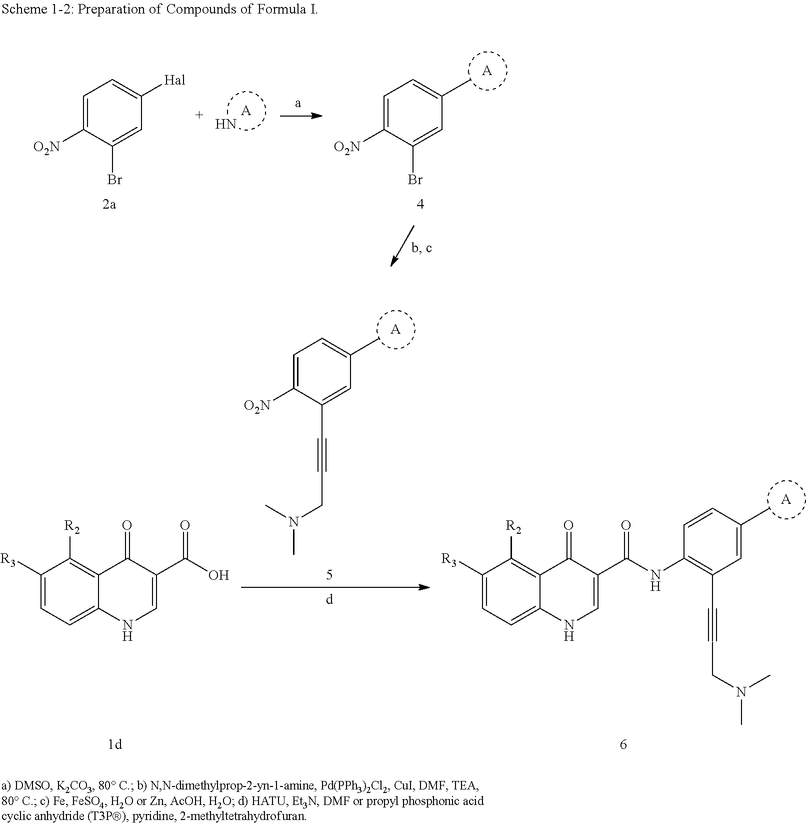

Scheme 1-2 depicts the synthesis of compounds of Formula I bearing a propynyl amine sidechain. Thus, coupling of nitrobenzene 2a, wherein Hal is bromide, chloride, or the like, with

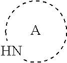

3 as defined herein in the presence potassium carbonate in DMSO provides compound 4. Palladium-catalyzed coupling of compound 4 with N,N-dimethylprop-2-yn-1-amine, followed by iron or zinc catalyzed reduction of the nitro moiety, provides amine 5. Coupling of amine 5 with carboxylic acid 1d provides compound 6 which is a compound of Formula I.

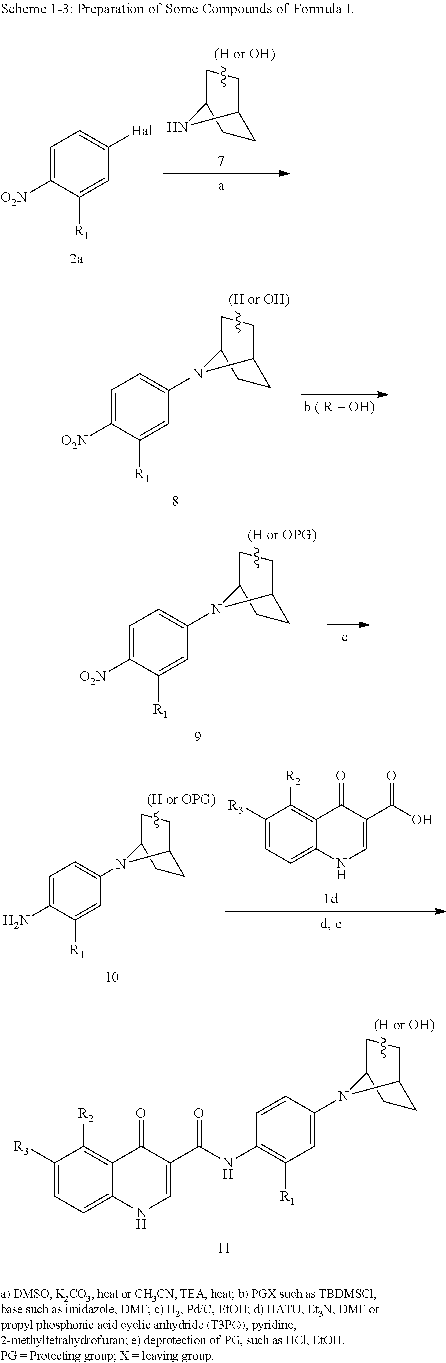

Scheme 1-3 depicts the synthesis of a compound of Formula I wherein

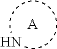

3 is 7-azabicyclo[2.2.1]heptane, optionally bearing an exo or endo hydroxy group at the 2-position. The hydroxy-substituted adducts (+)-endo-7-azabicyclo[2.2.1]heptan-2-ol, (−)-endo-7-azabicyclo[2.2.1]heptan-2-ol, (+)-exo-7-azabicyclo[2.2.1]heptan-2-ol, and (−)-exo-7-azabicyclo[2.2.1]heptan-2-ol can be prepared using procedures as described in Fletcher, S. R., et al., “Total Synthesis and Determination of the Absolute Configuration of Epibatidine,” J. Org. Chem., 59, pp. 1771-1778 (1994). 7-Azabicyclo[2.2.1]heptane itself is commercially available from Tyger Scientific Inc. 324 Stokes Avenue Ewing, N.J., 08638 USA.

Thus, as with the series of transformations summarized in Schemes 1-1 and 1-2, coupling of compound 2a with the bicyclo[2.2.1]amine of Formula 7 provides a compound of Formula 8. If the compound of Formula 8 has a hydroxy group, it may be necessary to protect the hydroxy group with a protecting group, such as a silyl protecting group as in step b, prior to subsequent transformations. Treatment of the hydroxylated compound of Formula 8 with a silylating agent such as tert-butyl dimethylsilyl chloride, using known conditions, provides the protected compound of Formula 9. Reduction of the nitro moiety provides an amine of Formula 10. Amide formation with 1d (cf. Scheme 1-3) and removal of the hydroxy protecting group (step e—as needed) provides a compound of Formula 11 which is also a compound of Formula I.

II.A.3.b. Embodiments of the Process for Making Compounds of Formula I

Another aspect of the invention relates to a process for preparing a compound of Formula (Ic):

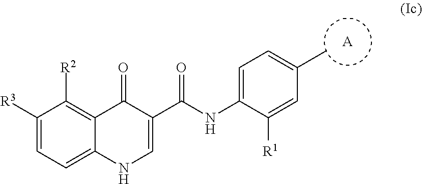

or pharmaceutically acceptable salts thereof, wherein the process comprises:

(a) reacting the acid of formula 1d with an amine of formula 2c to provide a compound of Formula (Ic)

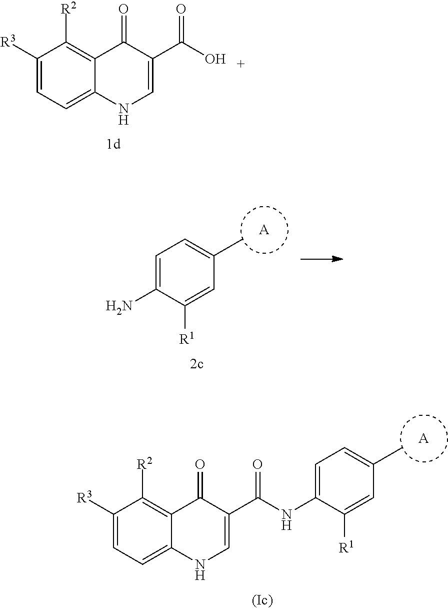

wherein:

Ring A is selected from:

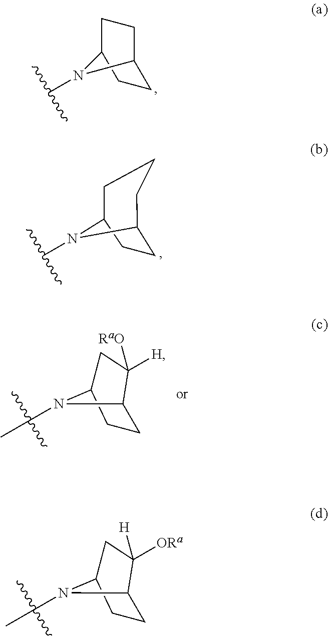

wherein
R¹ is —CF₃, —CN, or —C≡CCH₂N(CH₃)₂;
R² is hydrogen, —CH₃, —CF₃, —OH, or —CH₂OH;
R³ is hydrogen, —CH₃, —OCH₃, or —CN;
   provided that both R² and R³ are not simultaneously hydrogen, and
R^{a} is hydrogen or a silyl protecting group selected from the group consisting of trimethylsilyl (TMS), tert-butyldiphenylsilyl (TBDPS), tert-butyldimethylsilyl (TBDMS), triisopropylsilyl (TIPS), and [2-(trimethylsilyl)ethoxy]methyl (SEM).

In one embodiment, the reaction of the acid of formula 1d with the amine of formula 2c occurs in a solvent in the presence of O-(7-azabenzotriazol-1-yl)-N,N,N′,N′-tetramethyluronium hexafluorophosphate (HATU) and triethylamine or in a solvent in the presence of propyl phosphonic acid cyclic anhydride (T3P®) and pyridine. More particularly, the solvent comprises N,N-dimethyl formamide, ethyl acetate, or 2-methyltetrahydrofuran.

In another embodiment, R^{a} is hydrogen or TBDMS.

In another embodiment, R^{a} is TBDMS.

In another embodiment, the process comprises a further deprotection step; for instance, when ring A is

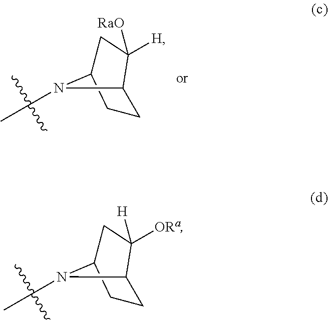

wherein R^{a} is a silyl protecting group, to generate a compound of Formula (Ic), wherein ring A is

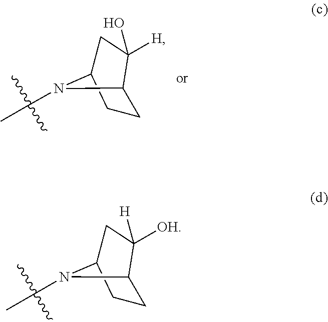

Typically, removal of a silyl protecting group requires treatment with acid such as acetic acid or a dilute mineral acid or the like, although other reagents, such as a source of fluoride ion (e.g., tetrabutylammonium fluoride), may be used.

In the process, the amine of formula 2c is prepared from a compound of formula 2a comprising the steps of:
(a) reacting the compound of formula 2a with an amine of formula 3 to provide the compound of formula 2b

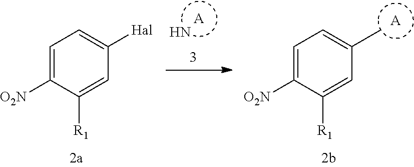

wherein:
Hal is F, Cl, Br, or I; and the amine of formula 3 is

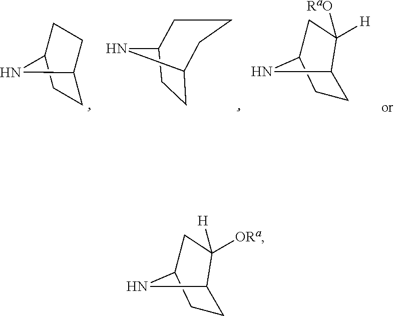

and
(b) reducing the compound of formula 2b to the amine of formula 2c.

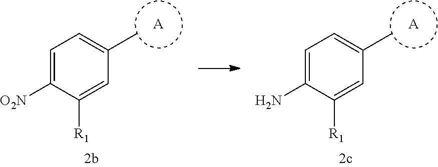

In one embodiment of the process for making the amine of formula 2c, the amine of formula 3 in step (a) is generated in situ from the corresponding quaternary ammonium salt, such as an amine hydrochloride salt, although other ammonium salts (e.g. the trifluoracetate salt), may be used as well.

In one embodiment of step (a) for forming the amine of formula 2c, when the amine of formula 3 is

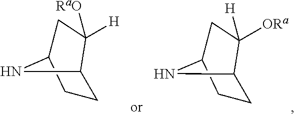

R^{a} is hydrogen or TBDMS. More particularly, R^{a} is TBDMS.

In another embodiment, step (a) occurs in a polar aprotic solvent in the presence of a tertiary amine base. Examples of tertiary amines that can be employed include triethylamine, diisopropylethyl amine, 1,5-diazabicyclo[4.3.0]non-5-ene (DBN), 1,8-diazabicyclo[5.4.0]undec-7-ene (DBU), 1,4-diazabicyclo[2.2.2]octane (DABCO) and pyridine. Examples of solvents that can be employed include N,N-dimethyl formamide, dimethyl sulfoxide or acetonitrile.

In one embodiment, the tertiary amine base is triethylamine.

In another embodiment, step (a) occurs in acetonitrile in the presence of triethylamine.

In another embodiment, the reaction temperature of step (a) is between approximately 75° C. and approximately 85° C.

In another embodiment, the reaction time for step (a) is between approximately 2 and approximately 30 hours.

In one embodiment of the process for making the amine of formula 2c, step (b) occurs in a polar protic solvent or a mixture of polar protic solvents in the presence of a palladium catalyst. When palladium is the catalyst, the solvent in step (b) typically is a polar protic solvent such as an alcohol. More particularly, comprises methanol or ethanol.

In another embodiment, step (b) occurs in a polar protic solvent, such as water, in the presence of Fe and FeSO₄ or Zn and AcOH.

Another aspect of the invention relates to a process for preparing a compound of Formula (Ic):

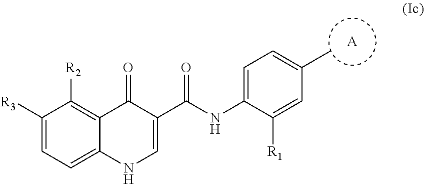

or pharmaceutically acceptable salts thereof, comprising the steps of:

(a) reacting a compound of formula 2a with an amine of formula 3 to provide a compound of formula 2b

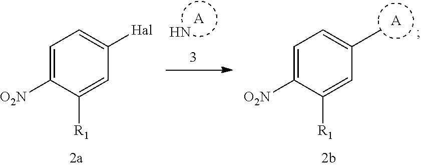

(b) converting the compound of formula 2b to the amine of formula 2c via reduction

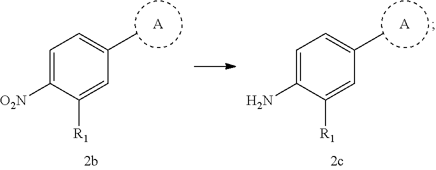

and

(c) reacting the amine of formula 2c with an acid of formula id to provide a compound of Formula (Ic)

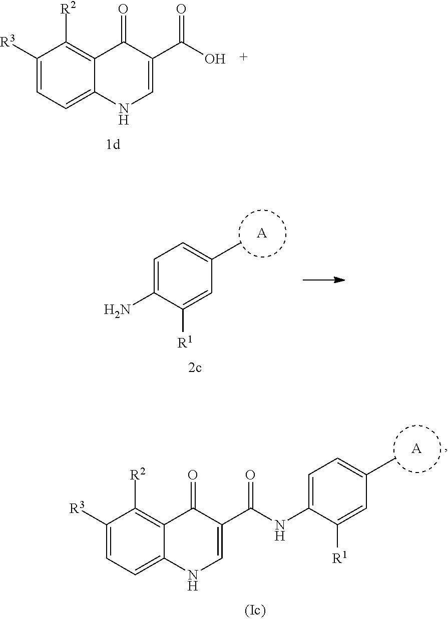

wherein Hal is F, Cl, Br, or I;

the amine of formula 3 is

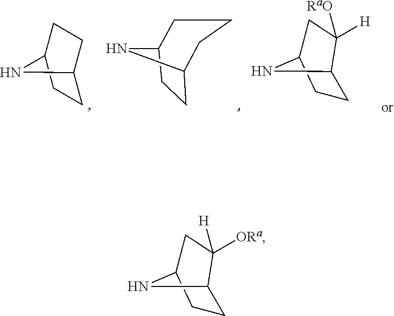

and ring A is selected from:

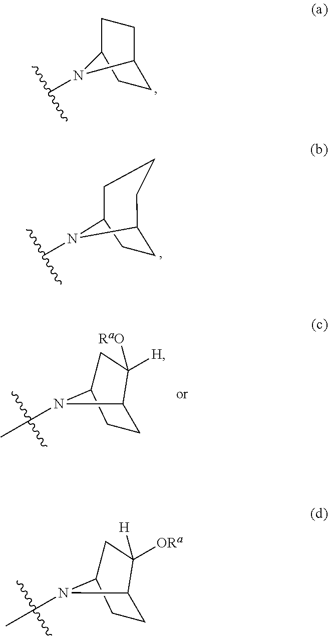

wherein
R¹ is —CF₃, —CN, or —C≡CH₂N(CH₃)₂;
R² is hydrogen, —CH₃, —CF₃, —OH, or —CH₂OH;
R³ is hydrogen, —CH₃, —OCH₃, or —CN;
   provided that both R² and R³ are not simultaneously hydrogen, and
R^{a} is hydrogen or a silyl protecting group selected from the group consisting of trimethylsilyl (TMS), tert-butyldiphenylsilyl (TBDPS), tert-butyldimethylsilyl (TBDMS), triisopropylsilyl (TIPS), and [2-(trimethylsilyl)ethoxy]methyl (SEM).

In one embodiment, the amine of formula 3 in step (a) is generated in situ from the corresponding quaternary ammonium salt, such as an amine hydrochloride salt, although other ammonium salts (e.g. the trifluoracetate salt), may be used as well.

In one embodiment of step (a) for forming the amine of formula 2c, when the amine of formula 3 is

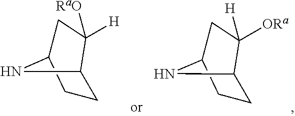

R^{a} is hydrogen or TBDMS. More particularly, R^{a} is TBDMS.

In another embodiment, step (a) occurs in a polar aprotic solvent in the presence of a tertiary amine base. Examples of tertiary amines that can be employed include triethylamine, diisopropylethyl amine, 1,5-diazabicyclo[4.3.0]non-5-ene (DBN), 1,8-diazabicyclo[5.4.0]undec-7-ene (DBU), 1,4-diazabicyclo[2.2.2]octane (DABCO) and pyridine.

In one embodiment, the tertiary amine base is triethylamine.

In another embodiment, step (a) occurs in acetonitrile in the presence of triethylamine.

In another embodiment, the reaction temperature of step (a) is between approximately 75° C. and approximately 85° C.

In another embodiment, the reaction time for step (a) is between approximately 2 and approximately 30 hours.

In one embodiment of the process for making the amine of formula 2c, step (b) occurs in a polar protic solvent or a mixture of polar protic solvents in the presence of a palladium catalyst. When palladium is the catalyst, the solvent in step (b) typically is a polar protic solvent such as an alcohol. More particularly, the solvent comprises methanol or ethanol.

In another embodiment, step (b) occurs in a polar protic solvent, such as water, in the presence of Fe and FeSO₄ or Zn and AcOH.

In one embodiment of step (c), the reaction of the acid of formula 1d with the amine of formula 2c occurs in a solvent in the presence of O-(7-azabenzotriazol-1-yl)-N,N,N′,N′-tetramethyluronium hexafluorophosphate (HATU) and triethylamine or in a solvent in the presence of propyl phosphonic acid cyclic anhydride (T3P®) and pyridine. More particularly, the solvent comprises N,N-dimethyl formamide, ethyl acetate, or 2-methyltetrahydrofuran.

In another embodiment, R^{a} is hydrogen or TBDMS.

In another embodiment, R^{a} is TBDMS.

In another embodiment, the process comprises a further deprotection step; for instance, when ring A is

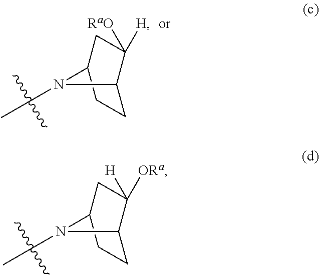

wherein R^{a} is a silyl protecting group, to generate a compound of Formula (I), wherein ring A is

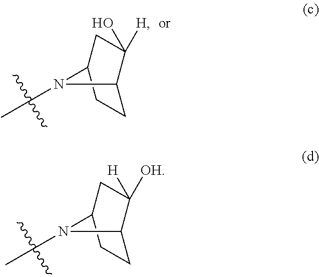

Typically, removal of a silyl protecting group requires treatment with acid such as acetic acid or a dilute mineral acid or the like, although other reagents, such as a source of fluoride ion (e.g., tetrabutylammonium fluoride), may be used.

Another aspect of the invention relates to a compound which is

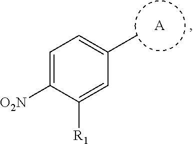

wherein ring A is

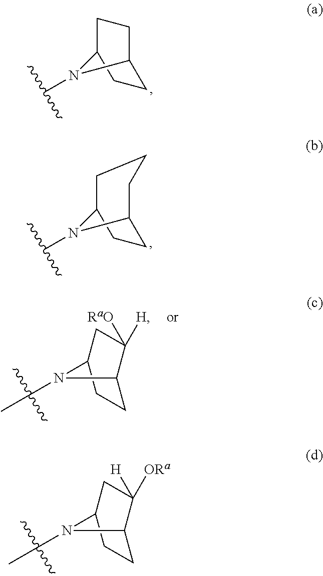

wherein

R¹ is —CF₃, —CN, or —C≡CCH₂N(CH₃)₂, and

R^{a} is a silyl protecting group selected from the group consisting of trimethylsilyl (TMS), tert-butyldiphenylsilyl (TBDPS), tert-butyldimethylsilyl (TBDMS), triisopropylsilyl (TIPS), and [2-(trimethylsilyl)ethoxy]methyl (SEM).

Another aspect of the invention relates to a compound which is

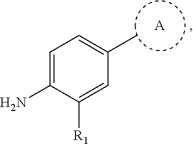

wherein ring A is

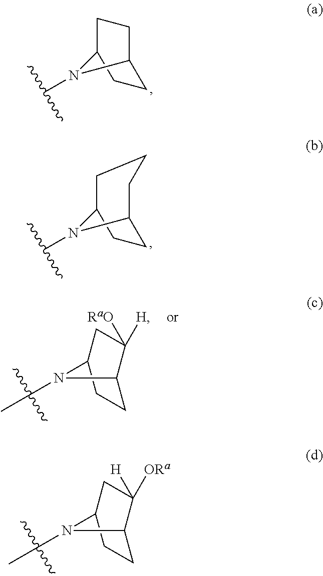

wherein

R¹ is —CF₃, —CN, or —C≡CCH₂N(CH₃)₂, and

R^{a} is a silyl protecting group selected from the group consisting of trimethylsilyl (TMS), tert-butyldiphenylsilyl (TBDPS), tert-butyldimethylsilyl (TBDMS), triisopropylsilyl (TIPS), and [2-(trimethylsilyl)ethoxy]methyl (SEM).

Another aspect of the invention relates to a compound of Formula (IA):

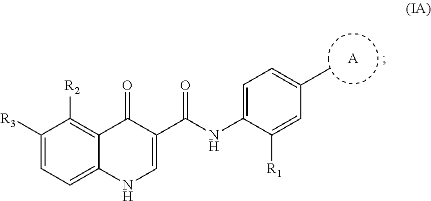

or pharmaceutically acceptable salts thereof, wherein:

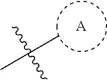

is selected from

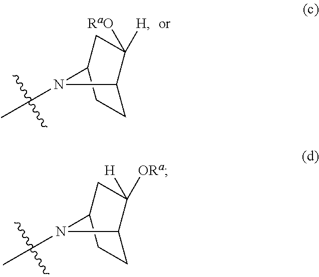

wherein
R¹ is —CF₃, —CN, or —C≡CH₂N(CH₃)₂;
R² is hydrogen, —CH₃, —CF₃, —OH, or —CH₂OH;
R³ is hydrogen, —CH₃, —OCH₃, or —CN;
   provided that both R² and R³ are not simultaneously hydrogen, and
R^{a} is a silyl protecting group selected from the group consisting of trimethylsilyl (TMS), tert-butyldiphenylsilyl (TBDPS), tert-butyldimethylsilyl (TBDMS), triisopropylsilyl (TIPS), and [2-(trimethylsilyl)ethoxy]methyl (SEM).

Another aspect of the invention relates to a compound of Formula (I)

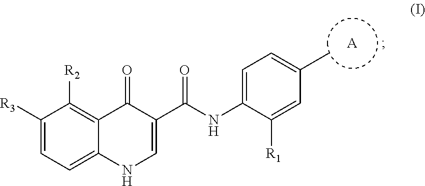

or pharmaceutically acceptable salts thereof, wherein:

Ring A is selected from:

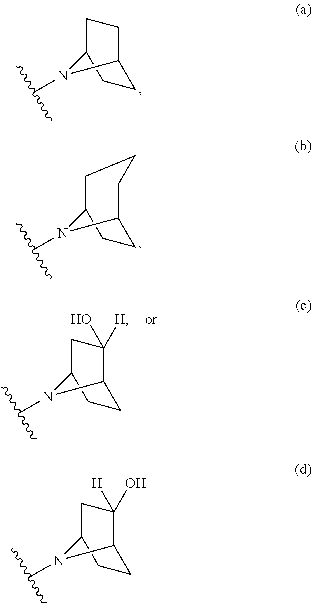

wherein

R¹ is —CF₃, —CN, or —C≡CCH₂N(CH₃)₂;

R² is hydrogen, —CH₃, —CF₃, —OH, or —CH₂OH;

R³ is hydrogen, —CH₃, —OCH₃, or —CN;
provided that both R² and R³ are not simultaneously hydrogen;

made by any of the processes disclosed herein.

Another aspect of the invention relates to a compound selected from the group consisting of:

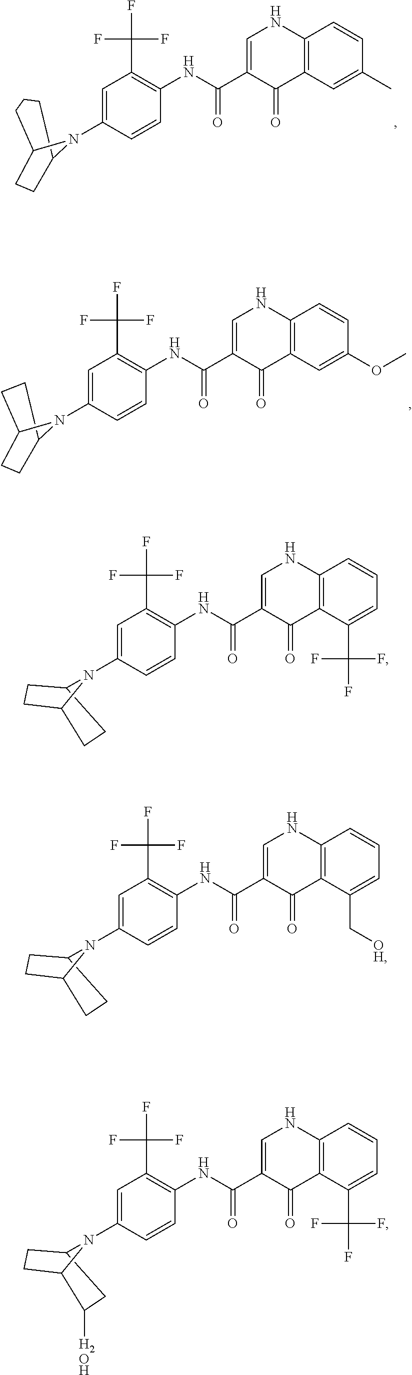

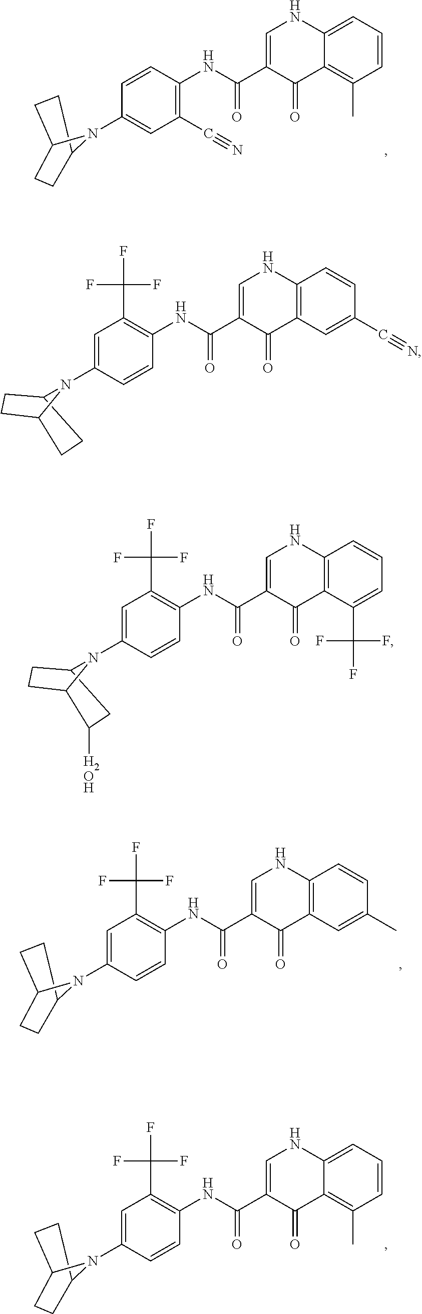

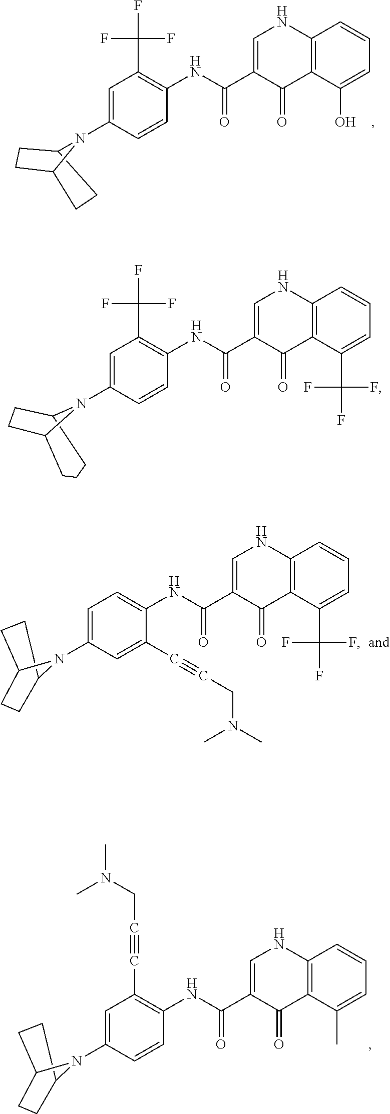

made by any of the processes disclosed herein.
II.A.3.c. Examples

Intermediate 1: 4-oxo-5-(trifluoromethyl)-1,4-dihydroquinoline-3-carboxylic acid (17). The synthesis of the title compound is depicted in Scheme 1-4.

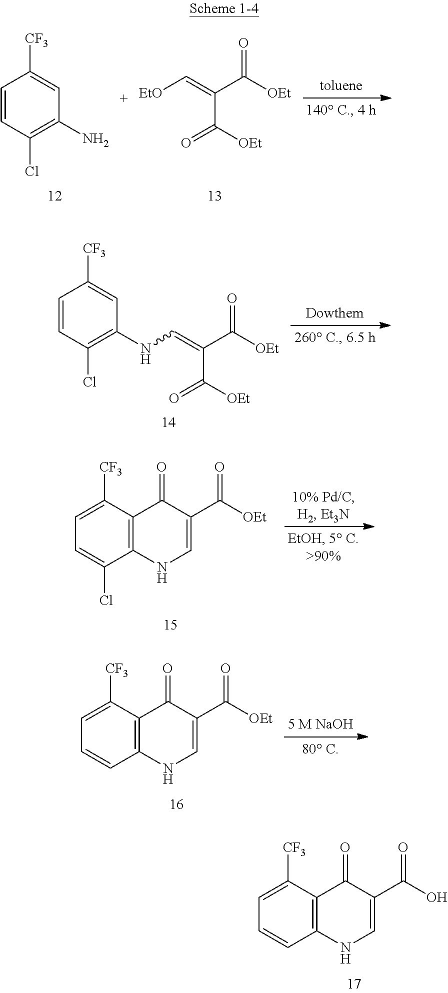

Preparation of diethyl 2-((2-chloro-5-(trifluoromethyl)phenylamino) methylene) malonate (14). 2-Chloro-5-(trifluoromethyl)aniline 12 (200 g, 1.023 mol), diethyl 2-(ethoxymethylene)malonate 13 (276 g, 1.3 mol) and toluene (100 mL) were combined under a nitrogen atmosphere in a three-neck, 1-L round bottom flask equipped with Dean-Stark condenser. The solution was heated with stirring to 140° C. and the temperature was maintained for 4 h. The reaction mixture was cooled to 70° C. and hexane (600 mL) was slowly added. The resulting slurry was stirred and allowed to warm to room temperature. The solid was collected by filtration, washed with 10% ethyl acetate in hexane (2×400 mL) and then dried under vacuum to provide a white solid (350 g, 94% yield) as the desired condensation product diethyl 2-((2-chloro-5-(trifluoromethyl)phenylamino) methylene) malonate 14. ¹H NMR (400 MHz, DMSO-d₆) δ 11.28 (d, J=13.0 Hz, 1H), 8.63 (d, J=13.0 Hz, 1H), 8.10 (s, 1H), 7.80 (d, J=8.3 Hz, 1H), 7.50 (dd, J=1.5, 8.4 Hz, 1H), 4.24 (q, J=7.1 Hz, 2H), 4.17 (q, J=7.1 Hz, 2H), 1.27 (m, 6H).

Preparation of ethyl 8-chloro-4-oxo-5-(trifluoromethyl)-1,4-dihydroquinoline-3-carboxylate (15). A 3-neck, 1-L flask was charged with Dowtherm® (200 mL, 8 mL/g), which was degassed at 200° C. for 1 h. The solvent was heated to 260° C. and charged in portions over 10 min with diethyl 2-((2-chloro-5-(trifluoromethyl)phenylamino) methylene)malonate 14 (25 g, 0.07 mol). The resulting mixture was stirred at 260° C. for 6.5 hours (h) and the resulting ethanol byproduct removed by distillation. The mixture was allowed to slowly cool to 80° C. Hexane (150 mL) was slowly added over 30 minutes (min), followed by an additional 200 mL of hexane added in one portion. The slurry was stirred until it had reached room temperature. The solid was filtered, washed with hexane (3×150 mL), and then dried under vacuum to provide ethyl 8-chloro-4-oxo-5-(trifluoromethyl)-1,4-dihydroquinoline-3-carboxylate 15 as a tan solid (13.9 g, 65% yield). ¹H NMR (400 MHz, DMSO-d₆) δ 11.91 (s, 1H), 8.39 (s, 1H), 8.06 (d, J=8.3 Hz, 1H), 7.81 (d, J=8.4 Hz, 1H), 4.24 (q, J=7.1 Hz, 2H), 1.29 (t, J=7.1 Hz, 3H).

Preparation of ethyl 4-oxo-5-(trifluoromethyl)-1H-quinoline-3-carboxylate (16). A 3-neck, 5-L flask was charged with of ethyl 8-chloro-4-oxo-5-(trifluoromethyl)-1,4-dihydroquinoline-3-carboxylate 15 (100 g, 0.3 mol), ethanol (1250 mL, 12.5 mL/g) and triethylamine (220 mL, 1.6 mol). The vessel was then charged with 10 g of 10% Pd/C (50% wet) at 5° C. The reaction was stirred vigorously under hydrogen atmosphere for 20 h at 5° C., after which time the reaction mixture was concentrated to a volume of approximately 150 mL. The product, ethyl 4-oxo-5-(trifluoromethyl)-1H-quinoline-3-carboxylate 16, as a slurry with Pd/C, was taken directly into the next step.

Preparation of 4-oxo-5-(trifluoromethyl)-1,4-dihydroquinoline-3-carboxylic acid (17). Ethyl 4-oxo-5-(trifluoromethyl)-1H-quinoline-3-carboxylate 16 (58 g, 0.2 mol, crude reaction slurry containing Pd/C) was suspended in NaOH (814 mL of 5 M, 4.1 mol) in a 1-L flask with a reflux condenser and heated at 80° C. for 18 h, followed by further heating at 100° C. for 5 h. The reaction was filtered warm through packed Celite to remove Pd/C and the Celite was rinsed with 1 N NaOH. The filtrate was acidified to about pH 1 to obtain a thick, white precipitate. The precipitate was filtered then rinsed with water and cold acetonitrile. The solid was then dried under vacuum to provide 4-oxo-5-(trifluoromethyl)-1,4-dihydroquinoline-3-carboxylic acid 17 as a white solid (48 g, 92% yield). ¹H NMR (400.0 MHz, DMSO-d₆) δ 15.26 (s, 1H), 13.66 (s, 1H), 8.98 (s, 1H), 8.13 (dd, J=1.6, 7.8 Hz, 1H), 8.06-7.99 (m, 2H).

Intermediate 2: 4-(7-azabicyclo[2.2.1]heptan-7-yl)-2-(trifluoromethyl)aniline (21). The synthesis of the title compound is depicted in Scheme 1-5.

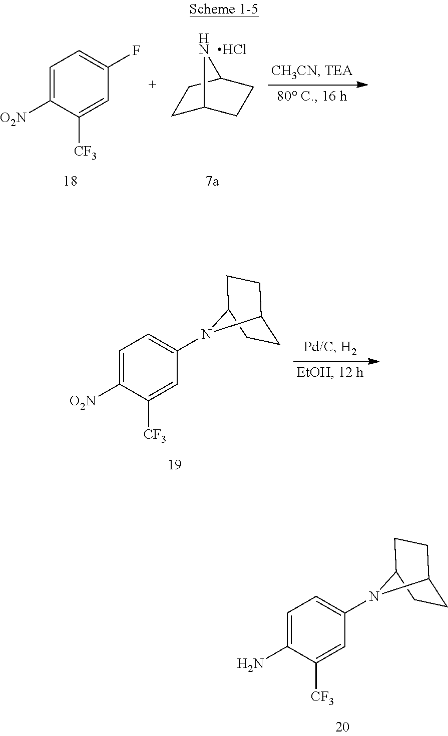

Preparation of 7-[4-nitro-3-(trifluoromethyl)phenyl]-7-azabicyclo[2.2.1]heptane (20). To a flask containing 7-azabicyclo[2.2.1]heptane hydrochloride 7a (4.6 g, 34.43 mmol, obtained from Tyger Scientific Inc., 324 Stokes Avenue, Ewing, N.J., 08638 USA under a nitrogen atmosphere was added a solution of 4-fluoro-1-nitro-2-(trifluoromethyl)benzene 18 (6.0 g, 28.69 mmol) and triethylamine (8.7 g, 12.00 mL, 86.07 mmol) in acetonitrile (50 mL). The reaction flask was heated at 80° C. under a nitrogen atmosphere for 16 h. The reaction mixture was allowed to cool and then was partitioned between water and dichloromethane. The organic layer was washed with 1 M HCl, dried over Na₂SO₄, filtered, and concentrated to dryness. Purification by silica gel chromatography (0-10% ethyl acetate in hexanes) yielded 7-[4-nitro-3-(trifluoromethyl)phenyl]-7-azabicyclo[2.2.1]heptane 19 (7.2 g, 88% yield) as a yellow solid. ¹H NMR (400.0 MHz, DMSO-d₆) δ 8.03 (d, J=9.1 Hz, 1H), 7.31 (d, J=2.4 Hz, 1H), 7.25 (dd, J=2.6, 9.1 Hz, 1H), 4.59 (s, 2H), 1.69-1.67 (m, 4H), 1.50 (d, J=7.0 Hz, 4H).

Preparation of 4-(7-azabicyclo[2.2.1]heptan-7-yl)-2-(trifluoromethyl)aniline (20). A flask charged with 7-[4-nitro-3-(trifluoromethyl)phenyl]-7-azabicyclo[2.2.1]heptane 19 (7.07 g, 24.70 mmol) and 10% Pd/C (0.71 g, 6.64 mmol) was evacuated and then flushed with nitrogen. Ethanol (22 mL) was added and the reaction flask was fitted with a hydrogen balloon. After stirring vigorously for 12 h, the reaction mixture was purged with nitrogen and Pd/C was removed by filtration. The filtrate was concentrated to a dark oil under reduced pressure and the residue purified by silica gel chromatography (0-15% ethyl acetate in hexanes) to provide 4-(7-azabicyclo[2.2.1]heptan-7-yl)-2-(trifluoromethyl)aniline 20 as a purple solid (5.76 g, 91% yield). ¹H NMR (400.0 MHz, DMSO-d₆) δ 6.95 (dd, J=2.3, 8.8 Hz, 1H), 6.79 (d, J=2.6 Hz, 1H), 6.72 (d, J=8.8 Hz, 1H), 4.89 (s, 2H), 4.09 (s, 2H), 1.61-1.59 (m, 4H) and 1.35 (d, J=6.8 Hz, 4H).

Intermediate 3: 2-amino-5-(7-azabicyclo[2.2.1]heptan-7-yl)benzonitrile (23). The synthesis of the title compound is depicted in Scheme 1-6.

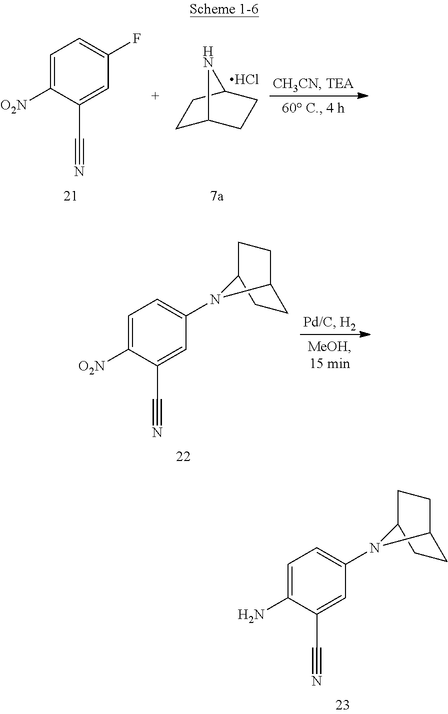

Preparation of 5-(7-azabicyclo[2.2.1]heptan-7-yl)-2-nitrobenzonitrile (22). To a solution of 5-fluoro-2-nitrobenzonitrile 21 (160 mg, 0.96 mmol) in acetonitrile (1 mL) was slowly added 7-azabicyclo[2.2.1]heptane hydrochloride 7a (129 mg, 0.96 mmol) and triethylamine (244 mg, 335.7 μL, 2.41 mmol). The reaction was stirred at 60° C. for 4 h. The reaction was quenched with water, acidified with 1 N HCl to pH 1, and extracted with dichloromethane (3×10 mL). The combined organic layers were washed with water, dried over MgSO₄, filtered and concentrated to provide 5-(7-azabicyclo[2.2.1]heptan-7-yl)-2-nitrobenzonitrile 22 (205 mg, 87% yield). LC/MS m/z 244.3 [M+H]⁺, retention time 1.69 min (RP-C₁₈, 10-99% CH₃CN/0.05% TFA over 3 min).

Preparation of 2-amino-5-(7-azabicyclo[2.2.1]heptan-7-yl)benzonitrile (23). A flask charged with 5-(7-azabicyclo[2.2.1]heptan-7-yl)-2-nitrobenzonitrile 22 (205 mg, 0.8427 mmol) and 10% Pd/C (41 mg, 0.39 mmol) was flushed with nitrogen and then evacuated under vacuum. Methanol (4 mL) was added under nitrogen atmosphere and the flask was fitted with a hydrogen balloon. After stirring for 15 min, the Pd/C was removed by filtration and solvent was removed under reduced pressure to provide 2-amino-5-(7-azabicyclo[2.2.1]heptan-7-yl)benzonitrile 23 (170 mg, 95% yield). ¹H NMR (400.0 MHz, DMSO-d₆) δ 7.02 (dd, J=2.8, 9.0 Hz, 1H), 6.87 (d, J=2.7 Hz, 1H), 6.68 (d, J=9.0 Hz, 1H), 5.36 (s, 2H), 4.09 (s, 2H), 1.59 (d, J=6.8 Hz, 4H), 1.34 (d, J=6.8 Hz, 4H).

Intermediate 4: 4-(7-azabicyclo[2.2.1]heptan-7-yl)-2-(3-(dimethylamino)prop-1-ynyl)aniline (27). The synthesis of the title compound is depicted in Scheme 1-7.

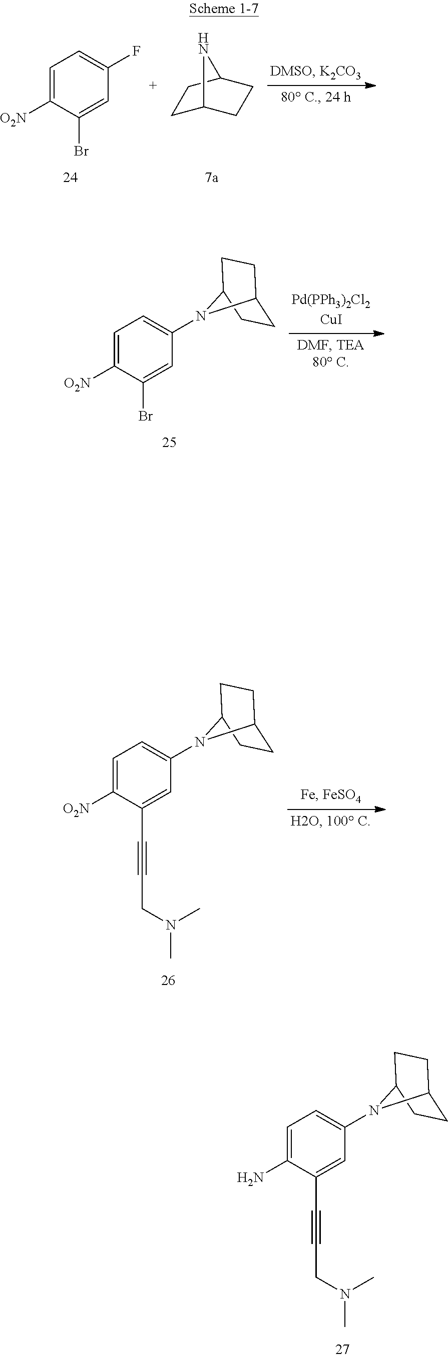

Preparation of 7-(3-bromo-4-nitrophenyl)-7-azabicyclo[2.2.1]heptane (25). To a solution of 2-bromo-4-fluoro-1-nitro-benzene 24 (1.1 g, 4.8 mmol) and K₂CO₃ (2.0 g, 14.3 mmol) in DMSO (8.400 mL) was added 7-azabicyclo[2.2.1]heptane 7a (765.4 mg, 5.7 mmol) portion-wise. The reaction was stirred at 80° C. for 24 h. The reaction was diluted with water to precipitate the product. The solid was redissolved in dichloromethane, washed with 1.0 N HCl, dried over MgSO₄, filtered and concentrated to provide 7-(3-bromo-4-nitrophenyl)-7-azabicyclo[2.2.1]heptane 25 (1.1 g, 78% yield). The crude product was used directly in the next step. LC/MS m/z 299.1 [M+H]⁺, retention time 1.97 min (RP-C₁₈, 10-99% CH₃CN/0.05% TFA over 3 min).

Preparation of 3-[5-(7-azabicyclo[2.2.1]heptan-7-yl)-2-nitro-phenyl]-N,N-dimethyl-prop-2-yn-1-amine (26). To 7-(3-bromo-4-nitro-phenyl)-7-azabicyclo[2.2.1]heptane 25 (500 mg, 1.683 mmol), Pd(PPh₃)₂Cl₂ (59 mg, 0.08 mmol), and cuprous iodide (9.616 mg, 1.708 μL, 0.05049 mmol) was added a solution of N,N-dimethylprop-2-yn-1-amine (420 mg, 538 μL, 5.05 mmol) in degassed DMF (5 mL) and triethylamine (5 mL). The reaction mixture was microwaved under N₂ for 10 min at 100° C. The reaction was diluted with ethyl acetate, washed with 50% saturated sodium bicarbonate solution (2×20 mL), water, and brine. The solution was dried over anhydrous Na₂SO₄ and filtered, leaving a red solid. Purification by silica gel chromatography (0-50% dichloromethane in ethyl acetate) yielded 3-[5-(7-azabicyclo[2.2.1]heptan-7-yl)-2-nitro-phenyl]-N,N-dimethyl-prop-2-yn-1-amine 26 (400 mg, 79% yield). LC/MS m/z 300.5 [M+H]⁺, retention time 1.11 min (RP-C₁₈, 10-99% CH₃CN/0.05% TFA over 3 min).

Preparation of 4-(7-azabicyclo[2.2.1]heptan-7-yl)-2-(3-dimethylaminoprop-1-ynyl)aniline (27). 3-[5-(7-Azabicyclo[2.2.1]heptan-7-yl)-2-nitro-phenyl]-N,N-dimethyl-prop-2-yn-1-amine 26 (340 mg, 1.14 mmol), iron (634 mg, 11.36 mmol) and ferrous sulfate heptahydrate (316 mg, 1.136 mmol) were suspended in water (1 mL) and refluxed for 20 min. The reaction was filtered and the solid washed with methanol and dichloromethane. The filtrate was concentrated and purified by silica gel chromatography using (0-5% methanol in dichloromethane) to provide 4-(7-azabicyclo[2.2.1]heptan-7-yl)-2-(3-dimethylaminoprop-1-ynyl)aniline 27 (148 mg, 48% yield). LC/MS m/z 270.3 [M+H]⁺, retention time 0.25 min (RP-C₁₈, 10-99% CH₃CN/0.05% TFA over 3 min).

Intermediate 5: exo-4-(2-(tert-butyldimethylsilyloxy)-7-azabicyclo[2.2.1]heptan-7-yl)-2-(trifluoromethyl)aniline (30). The synthesis of the title compound is depicted in Scheme 1-8.

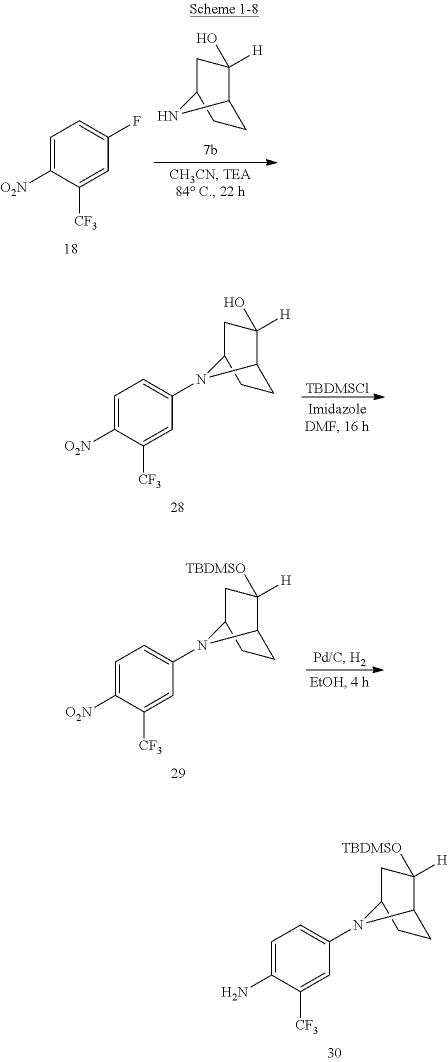

Preparation of exo-7-[4-nitro-3-(trifluoromethyl)phenyl]-7-azabicyclo[2.2.1]heptan-5-ol (28). To a flask containing exo-7-azabicyclo[2.2.1]heptan-2-ol 7b (0.86 g, 5.74 mmol) under a nitrogen atmosphere was added a solution of 4-fluoro-1-nitro-2-(trifluoromethyl)benzene 18 (1 g, 4.78 mmol) and triethylamine (1.45 g, 2.0 mL, 14.35 mmol) in acetonitrile (8 mL). The reaction was heated at 84° C. under a nitrogen atmosphere for 22 h. The reaction mixture was allowed to cool and then was partitioned between water and ethyl acetate. The layers were separated and the aqueous layer was extracted twice with ethyl acetate. The combined organic layers were dried over Na₂SO₄, filtered, and concentrated to dryness. Purification by silica gel chromatography (0-50% ethyl acetate in hexanes) provided exo-7-[4-nitro-3-(trifluoromethyl)phenyl]-7-azabicyclo[2.2.1]heptan-5-ol 28 as a yellow solid (0.67 g, 46% yield). LC/MS m/z 303.3 [M+H]⁺, retention time 1.51 min (RP-C₁₈, 10-99% CH₃CN/0.05% TFA over 3 min).

Preparation of exo-tert-butyl-dimethyl-[[7-[4-nitro-3-(trifluoromethyl)phenyl]-7-azabicyclo[2.2.1]heptan-5-yl]oxy]silane 29. Tert-butyl-chloro-dimethyl-silane (197 mg, 1.267 mmol) was added to a solution of 4H-imidazole (144 mg, 2.11 mmol) in DMF (0.5 mL). After the solution stopped bubbling, exo-7-[4-nitro-3-(trifluoromethyl)phenyl]-7-azabicyclo[2.2.1]heptan-5-ol 28 (255 mg, 0.84 mmol) was added as a solution in DMF (0.6 mL) and stirred at room temperature for 14 h. The reaction was quenched with water and extracted twice with diethyl ether, dried over MgSO₄, filtered and concentrated to a colorless oil. Purification by silica gel chromatography (0-40% dichloromethane in hexanes) afforded exo-tert-butyl-dimethyl-[[7-[4-nitro-3-(trifluoromethyl)phenyl]-7-azabicyclo[2.2.1]heptan-5-yl]oxy]silane 29 (318 mg, 90% yield) as a yellow oil. ¹H NMR (400.0 MHz, DMSO-d₆) δ 8.01 (d, J=9.2 Hz, 1H), 7.29 (d, J=2.4 Hz, 1H), 7.19 (dd, J=2.6, 9.2 Hz, 1H), 4.60 (t, J=4.4 Hz, 1H), 4.47 (d, J=5.2 Hz, 1H), 4.07 (dd, J=2.0, 6.8 Hz, 1H), 1.94 (dd, J=6.4, 12.8 Hz, 1H), 1.71-1.47 (m, 3H), 1.39-1.32 (m, 2H), 0.65 (s, 9H), 0.03 (s, 6H).

Preparation of exo-4-[5-[tert-butyl(dimethyl)silyl]oxy-7-azabicyclo[2.2.1]heptan-7-yl]-2-(trifluoromethyl)aniline (30). A flask containing palladium on activated carbon (10 wt %, 30 mg, 0.28 mmol) was evacuated, purged with N₂, and charged with a solution of exo-tert-butyl-dimethyl-[[7-[4-nitro-3-(trifluoromethyl)phenyl]-7-azabicyclo[2.2.1]heptan-5-yl]oxy]silane 29 (301 mg, 0.72 mmol) in ethanol (3 mL). The flask was evacuated and then was equipped with a balloon of H₂ and stirred for 4 h at room temperature. The mixture was filtered and concentrated to dryness to yield exo-4-[5-[tert-butyl(dimethyl)silyl]oxy-7-azabicyclo[2.2.1]heptan-7-yl]-2-(trifluoromethyl)aniline 30 (268 mg, 96% yield) as an off-white solid. ¹H NMR (400.0 MHz, DMSO-d₆) δ 6.92 (dd, J=2.4, 8.8 Hz, 1H), 6.77 (d, J=2.6 Hz, 1H), 6.70 (d, J=8.8 Hz, 1H), 4.84 (s, 2H), 4.11 (t, J=4.4 Hz, 1H), 3.91-3.89 (m, 2H), 1.82 (dd, J=7.1, 12.3 Hz, 1H), 1.54-1.39 (m, 3H), 1.20-1.16 (m, 2H), 0.79 (s, 9H), 0.02 (s, 6H).

Intermediate 6: endo-4-(2-(tert-butyldimethylsilyloxy)-7-azabicyclo[2.2.1]heptan-7-yl)-2-(trifluoromethyl)aniline (34). The preparation of the title compound is depicted in Scheme 1-9.

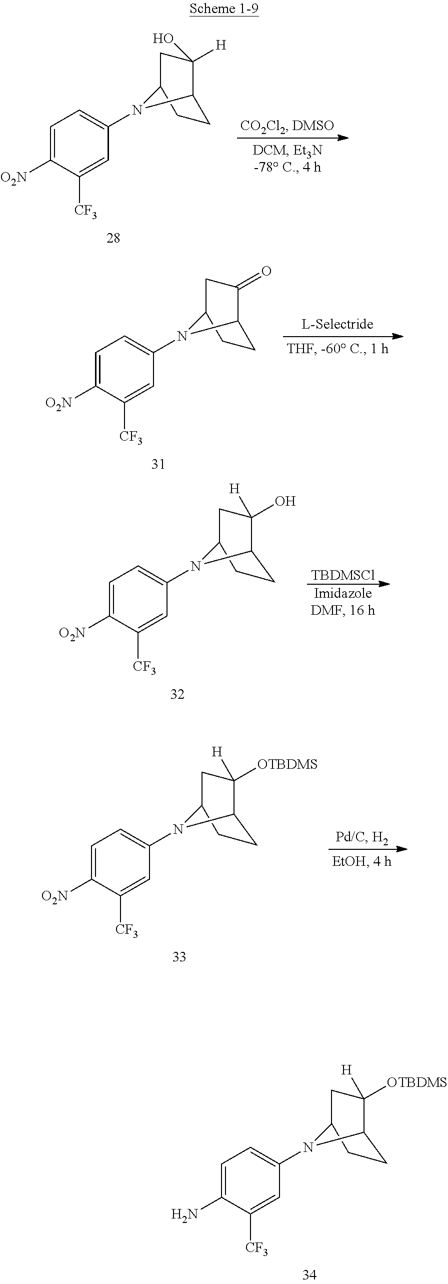

Preparation of 7-azabicyclo[2.2.1]heptan-5-one (31). To a solution of oxalyl dichloride (165 mg, 113 μL, 1.27 mmol) in dichloromethane (3 mL) under a nitrogen atmosphere at −78° C. was added a solution of DMSO (199 mg, 180 μL, 2.54 mmol) in dichloromethane (0.7 mL) dropwise. The reaction mixture was allowed to stir for 30 min and then a solution of exo-7-[4-nitro-3-(trifluoromethyl)phenyl]-7-azabicyclo[2.2.1]heptan-5-ol 28 (320 mg, 1.06 mmol) in dichloromethane (2.5 mL) was added dropwise. The reaction was stirred for an additional hour at −78° C., and then triethylamine (536 mg, 738 μL, 5.30 mmol) was added dropwise and the reaction was warmed to room temperature. The reaction mixture was diluted with dichloromethane, partitioned between dichloromethane and water, and the layers were separated. The aqueous layer was extracted once more with dichloromethane. The combined organic layers were dried over Na₂SO₄, filtered and concentrated to a yellow oil. Purification by silica gel chromatography (0-30% ethyl acetate in hexanes) provided 7-[4-nitro-3-(trifluoromethyl)phenyl]-7-azabicyclo[2.2.1]heptan-5-one 31 (266 mg, 84% yield) as a yellow solid. ¹H NMR (400.0 MHz, DMSO-d₆) δ 8.06 (d, J=9.1 Hz, 1H), 7.47 (d, J=2.4 Hz, 1H), 7.39 (dd, J=2.6, 9.1 Hz, 1H), 4.98 (t, J=4.5 Hz, 1H), 4.84 (d, J=5.4 Hz, 1H), 2.44 (d, J=3.1 Hz, 1H), 2.23 (d, J=16 Hz, 1H), 2.00-1.92 (m, 1H), 1.88-1.70 (m, 2H), 1.66-1.60 (m, 1H).

Preparation of endo-7-[4-nitro-3-(trifluoromethyl)phenyl]-7-azabicyclo[2.2.1]heptan-5-ol (32). To a solution of 7-[4-nitro-3-(trifluoromethyl)phenyl]-7-azabicyclo[2.2.1]heptan-5-one 31 (261 mg, 0.87 mmol) in THF (11 mL) at −55° C. under a nitrogen atmosphere was added a solution of lithium hydrido-trisec-butyl-boron (1.04 mL of 1 M, 1.04 mmol) dropwise. After 30 min, the reaction mixture was transferred to an ice water bath and stirring was continued. The reaction mixture was quenched with methanol (1.2 mL) at 0° C. The reaction mixture was partitioned between dichloromethane/water, separated and the aqueous layer was extracted twice more with dichloromethane. The organic layers were combined, dried over Na₂SO₄, filtered, and concentrated to dryness. Purification by silica gel chromatography (0-50% ethyl acetate in hexanes) provided endo-7-[4-nitro-3-(trifluoromethyl)phenyl]-7-azabicyclo[2.2.1]heptan-5-ol 32 (222 mg, 84% yield) as a yellow solid. ¹H NMR (400.0 MHz, DMSO-d₆) δ 8.01 (d, J=9.1 Hz, 1H), 7.27 (d, J=3.0 Hz, 1H), 7.22 (dd, J=2.6, 9.1 Hz, 1H), 5.17 (d, J=4.4 Hz, 1H), 4.49 (t, J=4.9 Hz, 1H), 4.44 (t, J=4.5 Hz, 1H), 4.16-4.10 (m, 1H), 2.20-2.06 (m, 2H), 1.67-1.44 (m, 3H), 1.09 (dd, J=3.5, 12.4 Hz, 1H).

Preparation of endo-tert-butyl-dimethyl-[[7-[4-nitro-3-(trifluoromethyl)phenyl]-7-azabicyclo[2.2.1]heptan-5-yl]oxy]silane (33). Tert-butylchlorodimethylsilane (168 mg, 1.08 mmol) was added to a solution of 4H-imidazole (122 mg, 1.80 mmol) in DMF (425.3 μL). After the solution stopped bubbling, endo-7-[4-nitro-3-(trifluoromethyl)phenyl]-7-azabicyclo[2.2.1]heptan-5-ol 32 (217 mg, 0.72 mmol) was added as a solution in DMF (1 mL) and stirred at room temperature for 14 h. The reaction was quenched with water and extracted twice with diethyl ether, dried over MgSO₄, filtered, and concentrated to a colorless oil. Purification by silica gel chromatography (0-40% dichloromethane in hexanes) afforded endo-tert-butyl-dimethyl-[[7-[4-nitro-3-(trifluoromethyl)phenyl]-7-azabicyclo[2.2.1]heptan-5-yl]oxy]silane 33 (251 mg, 84% yield) as a yellow oil. ¹H NMR (400.0 MHz, DMSO-d₆) δ 8.01 (d, J=9.1 Hz, 1H), 7.32 (d, J=2.3 Hz, 1H), 7.26 (dd, J=2.5, 9.1 Hz, 1H), 4.54-4.51 (m, 2H), 4.29-4.26 (m, 1H), 2.20-2.11 (m, 2H), 1.67-1.45 (m, 3H), 1.08 (dd, J=3.2, 12.4 Hz, 1H), 0.88 (s, 9H), 0.07 (d, J=2.6 Hz, 6H).

Preparation of endo-4-[5-[tert-butyl(dimethyl)silyl]oxy-7-azabicyclo[2.2.1]heptan-7-yl]-2-(trifluoromethyl)aniline (34). A flask containing palladium on activated carbon (10 wt %, 24 mg, 0.23 mmol) was evacuated and then purged under a nitrogen atmosphere. To this was added a solution of endo-tert-butyl-dimethyl-[[7-[4-nitro-3-(trifluoromethyl)phenyl]-7-azabicyclo[2.2.1]heptan-5-yl]oxy]silane 33 (240 mg, 0.58 mmol) in ethanol (5 mL). The reaction mixture was evacuated, then equipped with a balloon of H₂ and stirred for 4 h at room temperature. The mixture was filtered and concentrated to dryness to yield endo-4-[5-[tert-butyl(dimethyl)silyl]oxy-7-azabicyclo[2.2.1]heptan-7-yl]-2-(trifluoromethyl)aniline 34 (222 mg, 100% yield) as an off-white solid. ¹H NMR (400.0 MHz, DMSO-d₆) δ 6.95 (dd, J=2.4, 8.8 Hz, 1H), 6.79 (d, J=2.6 Hz, 1H), 6.72 (d, J=8.8 Hz, 1H), 4.91 (s, 2H), 4.24-4.19 (m, 1H), 4.06-4.03 (m, 2H), 2.12-1.99 (m, 2H), 1.55-1.53 (m, 1H), 1.42-1.36 (m, 2H), 0.96 (dd, J=3.2, 12.2 Hz, 1H), 0.87 (s, 9H), 0.05 (s, 6H).

### Example Compound 1

### N-(4-(7-azabicyclo[2.2.1]heptan-7-yl)-2-(trifluoromethyl)phenyl)-4-oxo-5-(trifluoromethyl)-1,4-dihydroquinoline-3-carboxamide

The preparation of the title compound is depicted in Scheme 1-10.

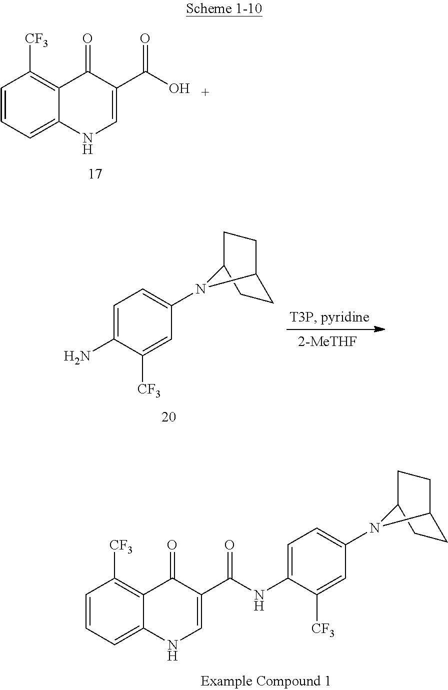

To a solution of 4-oxo-5-(trifluoromethyl)-1H-quinoline-3-carboxylic acid 17 (9.1 g, 35.39 mmol) and 4-(7-azabicyclo[2.2.1]heptan-7-yl)-2-(trifluoromethyl)aniline 20 (9.2 g, 35.74 mmol) in 2-methyltetrahydrofuran (91.00 mL) was added propyl phosphonic acid cyclic anhydride (T3P, 50% solution in ethyl acetate, 52.68 mL, 88.48 mmol) and pyridine (5.6 g, 5.73 mL, 70.78 mmol) at room temperature. The reaction flask heated at 65° C. for 10 h under a nitrogen atmosphere. After cooling to room temperature, the reaction was then diluted with ethyl acetate and quenched with saturated Na₂CO₃ solution (50 mL). The layers were separated, and the aqueous layer was extracted twice more with ethyl acetate. The combined organic layers were washed with water, dried over Na₂SO₄, filtered and concentrated to a tan solid. The crude solid product was slurried in ethyl acetate/diethyl ether (2:1), collected by vacuum filtration, and washed twice more with ethyl acetate/diethyl ether (2:1) to provide the product as a light yellow crystalline powder. The powder was dissolved in warm ethyl acetate and absorbed onto Celite. Purification by silica gel chromatography (0-50% ethyl acetate in dichloromethane) provided N-(4-(7-azabicyclo[2.2.1]heptan-7-yl)-2-(trifluoromethyl)phenyl)-4-oxo-5-(trifluoromethyl)-1,4-dihydroquinoline-3-carboxamide as a white crystalline solid (13.5 g, 76% yield). LC/MS m/z 496.0 [M+H]⁺, retention time 1.48 min (RP-C₁₈, 10-99% CH₃CN/0.05% TFA over 3 min). ¹H NMR (400.0 MHz, DMSO-d₆) δ 13.08 (s, 1H), 12.16 (s, 1H), 8.88 (s, 1H), 8.04 (dd, J=2.1, 7.4 Hz, 1H), 7.95-7.88 (m, 3H), 7.22 (dd, 2.5, 8.9 Hz, 1H), 7.16 (d, J=2.5 Hz, 1H), 4.33 (s, 2H), 1.67 (d, J=6.9 Hz, 4H), 1.44 (d, J=6.9 Hz, 4H).

### Example Compound 1 Form A HCl Salt N-(4-(7-azabicyclo[2.2.1]heptan-7-yl)-2-(trifluoromethyl)phenyl)-4-oxo-5-(trifluoromethyl)-1,4-dihydroquinoline-3-carboxamide hydrochloride (Form A-HCl)

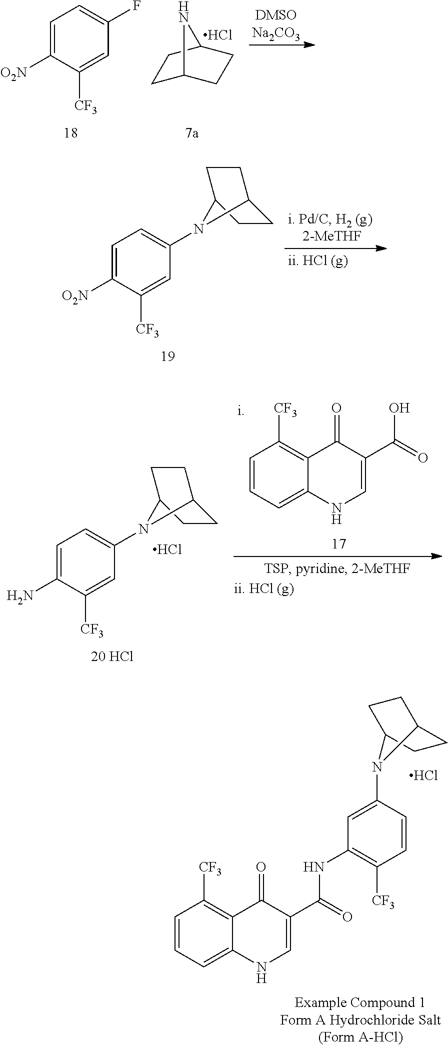

Preparation of 7-[4-nitro-3-(trifluoromethyl)phenyl]-7-azabicyclo[2.2.1]heptane (19). 4-Fluoro-1-nitro-2-(trifluoromethyl)benzene (18) (901 g) was added into a 30 L jacketed vessel. Sodium carbonate (959.1 g) and 5 L dimethylsulfoxide (DMSO) was added and the mixture was stirred under a nitrogen atmosphere. 7-azabicyclo[2.2.1]heptane hydrochloride (7a) (633.4 g) was added to the vessel in portions. The temperature of the mixture was gradually raised to 55° C., and the reaction was monitored by HPLC. When the substrate was less than 1% AUC, the reaction was considered complete. The mixture was then diluted with 10 vol. 2-Methyltetrahydrofuran and washed with 5.5 vol. water three times until no DMSO remained in the aqueous layer as determined by HPLC, to give 7-[4-nitro-3-(trifluoromethyl)phenyl]-7-azabicyclo[2.2.1]heptane (19) in 2-methyltetrahydrofuran (approximately 95% yield).

Preparation of hydrochloride salt of 4-(7-azabicyclo[2.2.1]heptan-7-yl)-2-(trifluoromethyl)aniline (20.HCl). Palladium on carbon (150 g, 5% w/w) was charged into a Büchi Hydrogenator (20 L capacity) under a nitrogen atmosphere, followed by the addition of 7-[4-nitro-3-(trifluoromethyl)phenyl]-7-azabicyclo[2.2.1]heptane (19) (1500 g) and 2-methyltetrahydrofuran (10.5 L, 7 vol). Hydrogen gas was charged into the closed hydrogenator to a pressure of 0.5 bar. A vacuum was applied for about 2 min, followed by the introduction of hydrogen gas to a pressure of 0.5 bar. This process was repeated 2 times. Hydrogen gas was then continuously charged to the mixture at a pressure of 0.5 bar. The mixture was then stirred at a temperature between 18 and 23° C. by cooling the vessel jacket. A vacuum was applied to the vessel when no more hydrogen gas was consumed and when there was no further exotherm. Nitrogen gas was then charged into the vessel at 0.5 bar and a vacuum was reapplied, followed by a second charge of 0.5 bar nitrogen gas. When the HPLC of a filtered aliquot showed that none of the 7-[4-nitro-3-(trifluoromethyl)phenyl]-7-azabicyclo[2.2.1]heptane (19) remained (e.g., ≦0.5%), the reaction mixture was transferred to a receiving flask under nitrogen atmosphere via a filter funnel using a Celite filter. The Celite filter cake was washed with 2-methyltetrahydrofuran (3 L, 2 vol). The washings and filtrate were charged into a vessel equipped with stirring, temperature control, and a nitrogen atmosphere. 4M HCl in 1,4-dioxane (1 vol) was added continuously over 1 h into the vessel at 20° C. The mixture was stirred for an additional 10 h (or overnight), filtered, and washed with 2-methyltetrahydrofuran (2 vol) and dried to generate 1519 g of the hydrochloride salt of 4-(7-azabicyclo[2.2.1]heptan-7-yl)-2-(trifluoromethyl)aniline (20.HCl) as a white crystalline solid (approximately 97% yield).

Alternative preparation of hydrochloride salt of 7-[4-amino-3-(trifluoromethyl)phenyl]-7-azabicyclo[2.2.1]heptane (20.HCl).

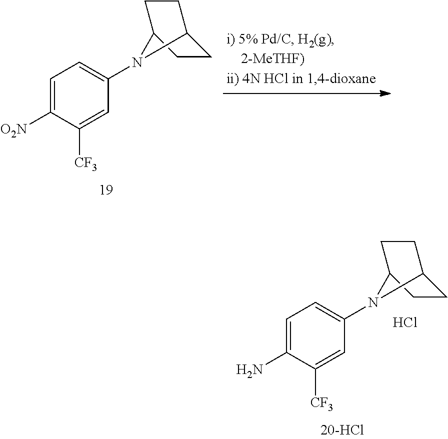

In a Büchi Hydrogenator (20 L capacity), palladium on carbon (5% w/w) (150 g) was introduced under nitrogen followed by the addition of 7-[4-nitro-3-(trifluoromethyl)phenyl]-7-azabicyclo[2.2.1]heptane 19 (1500 g) and 2-methyltetrahydrofuran (10.5 L, 7 vol). Hydrogen gas was charged into the vessel to a pressure of 0.5 bar. A vacuum was briefly applied (2 min), followed by the introduction of hydrogen gas to a pressure of 0.5 bar. This process was repeated 2 more times, and then hydrogen gas was charged to the hydrogenator continuously at 0.5 bar, and stirring was commenced. The temperature of the reaction mixture was maintained at 18 to 23° C. by cooling the vessel jacket. A vacuum was applied to the vessel when no more hydrogen gas was consumed and when there was no further exotherm. Nitrogen gas was then charged to the vessel, and a vacuum was re-applied, followed by a nitrogen gas charge at 0.5 bar. The reaction was deemed complete when an HPLC of a filtered aliquot showed that 7-[4-nitro-3-(trifluoromethyl)phenyl]-7-azabicyclo[2.2.1]heptane was not detected (≦0.5%). The reaction mixture was then filtered through Celite. The remaining slurry was transferred to a receiving flask under nitrogen gas via a filter funnel containing a Celite filter. The Celite cake was washed with 2-methyltetrahydrofuran (3 L, 2 vol). The filtrate and the washings were transferred to a vessel equipped with a stirring mechanism, temperature control, and a nitrogen atmosphere. 4M HCl in 1,4-dioxane (1 vol) was added continuously over 1 h to the vessel at 20° C. The resulting mixture was stirred for an additional 10 h, filtered and washed with 2-methyltetrahydrofuran (2 vol) and dried to generate 1519 g of 7-[4-amino-3-(trifluoromethyl)phenyl]-7-azabicyclo[2.2.1]heptane hydrochloride (20.HCl) as a white crystalline solid.

N-(4-(7-azabicyclo[2.2.1]heptan-7-yl)-2-(trifluoromethyl)phenyl)-4-oxo-5-(trifluoromethyl)-1,4-dihydroquinoline-3-carboxamide hydrochloride (Form A-HCl). 2-Methyltetrahydrofuran (0.57 L, 1.0 vol) was charged into a 30 L jacketed reactor vessel, followed by the addition of the hydrochloride salt of 4-(7-azabicyclo[2.2.1]heptan-7-yl)-2-(trifluoromethyl)aniline (20.HCl) (791 g, 2.67 mol) and 4-oxo-5-(trifluoromethyl)-1,4-dihydroquinoline-3-carboxylic acid (17) (573 g, 2.23 mol) and an additional 5.2 L (9.0 vol) of 2-methyltetrahydrofuran. Stirring commenced, and T3P in 2-methyltetrahydrofuran (2.84 kg, 4.46 mol) was added to the reaction mixture over 15 min. Pyridine (534.0 g, 546.0 mL, 6.68 mol) was then added via an addition funnel dropwise over 30 min. The mixture was warmed to 45° C. over about 30 min and stirred for 12-15 h. HPLC analysis indicated that 4-oxo-5-(trifluoromethyl)-1,4-dihydroquinoline-3-carboxylic acid was present in an amount less than 2%. The mixture was then cooled to room temperature. 2-Methyltetrahydrofuran (4 vol, 2.29 L) was added followed by water (6.9 vol, 4 L), while the temperature was maintained below 30° C. The water layer was removed and the organic layer was carefully washed twice with NaHCO₃ saturated aqueous solution. The organic layer was then washed with 10% w/w citric acid (5 vol) and finally with water (7 vol). The mixture was polished filtered and transferred into another dry vessel. Seed crystals of N-(4-(7-azabicyclo[2.2.1]heptan-7-yl)-2-(trifluoromethyl)phenyl)-4-oxo-5-(trifluoromethyl)-1,4-dihydroquinoline-3-carboxamide hydrochloride (Form A-HCl) (3.281 g, 5.570 mmol) from an earlier batch were added. HCl (g) (10 eq) was bubbled over 2 h and the mixture was stirred overnight. The resulting suspension was filtered, washed with 2-methyltetrahydrofuran (4 vol), suction dried and oven dried at 60° C. until constant weight generating 868 g of N-(4-(7-azabicyclo[2.2.1]heptan-7-yl)-2-(trifluoromethyl)phenyl)-4-oxo-5-(trifluoromethyl)-1,4-dihydroquinoline-3-carboxamide hydrochloride (Form A-HCl).

### Example Compound 1 Form B HCl Salt N-(4-(7-azabicyclo[2.2.1]heptan-7-yl)-2-(trifluoromethyl)phenyl)-4-oxo-5-(trifluoromethyl)-1,4-dihydroquinoline-3-carboxamide hydrochloride (Form B-HCl)

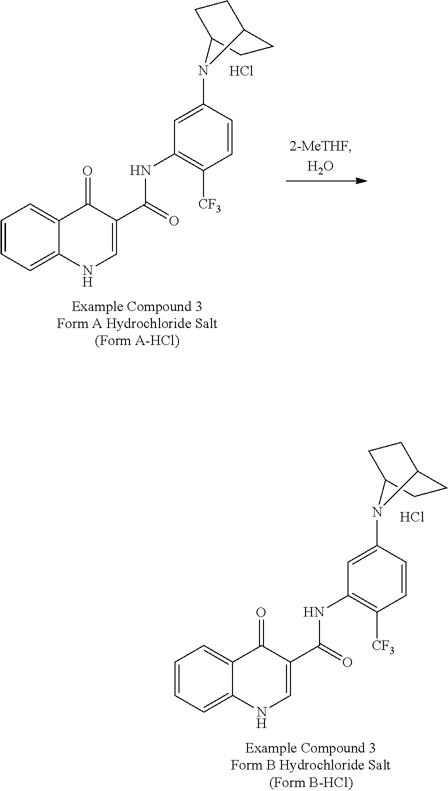

2-Methyltetrahydrofuran (100 mL) was charged into a 3-necked flask having a nitrogen atmosphere equipped with a stirrer. Example Compound 3 Form A-HCl (Example 3B, 55 g, 0.103 mol) was added to the flask, followed by 349 mL of 2-methyltetrahydrofuran, and stirring commenced. 28 mL of water was added into the flask and the flask was warmed to an internal temperature of 60° C. and stirred for 48 h. The flask was cooled to room temperature and stirred for 1 h. The reaction mixture was vacuum filtered until the filter cake was dry. The solid filter cake was washed with 2-methyltetrahydrofuran (4 vol) twice. The solid filter cake remained under vacuum suction for a period of about 30 minutes and was transferred to a drying tray. The filter cake was dried to a constant weight under vacuum at 60° C., to give Example Compound 3 Form B-HCl as a white crystalline solid (49 g) (approximately 90% yield).

### Example Compound 1-6

### Preparation of N-(4-(7-azabicyclo[2.2.1]heptan-7-yl)-2-cyanophenyl)-5-methyl-4-oxo-1,4-dihydroquinoline-3-carboxamide

The preparation of the title compound is depicted in Scheme 1-11.

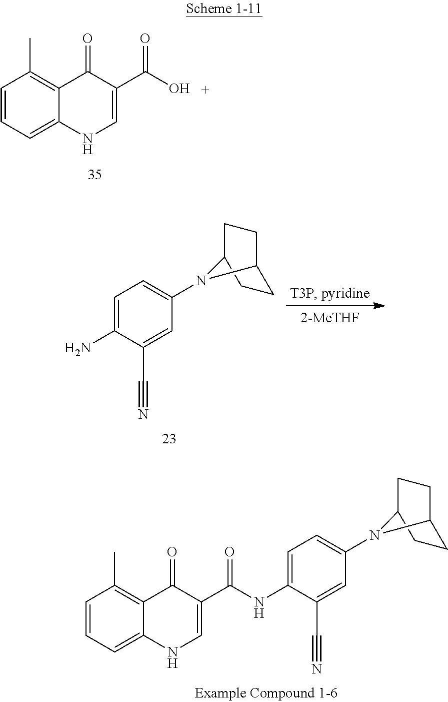

To a solution of 5-methyl-4-oxo-1,4-dihydroquinoline-3-carboxylic acid 35 (162 mg, 0.80 mmol) and 2-amino-5-(7-azabicyclo[2.2.1]heptan-7-yl)benzonitrile 23 (170 mg, 0.80 mmol) in 2-methyltetrahydrofuran (1.5 mL) was added propyl phosphonic acid cyclic anhydride (50% solution in ethyl acetate, 949.5 μL, 1.605 mmol) and pyridine (126 mg, 129 μL, 1.60 mmol). The reaction was capped and heated at 100° C. for 65 min with microwave irradiation. The reaction was cooled to room temperature, diluted with ethyl acetate (10 mL), and quenched with saturated Na₂CO₃ solution (6 mL). The organic layer was dried over Na₂SO₄, filtered and concentrated. Purification by silica gel chromatography (0-35% ethyl acetate in dichloromethane) provided N-(4-(7-azabicyclo[2.2.1]heptan-7-yl)-2-cyanophenyl)-5-methyl-4-oxo-1,4-dihydroquinoline-3-carboxamide (157 mg, 49% yield). LC/MS m/z 399.3 [M+H]⁺, retention time 1.47 min (RP-C₁₈, 10-99% CH₃CN/0.05% TFA over 3 min). ¹H NMR (400.0 MHz, DMSO-d₆) δ 12.77 (s, 1H), 12.75 (s, 1H), 8.77 (s, 1H), 8.11 (d, J=9.1 Hz, 1H), 7.64-7.60 (m, 1H), 7.55 (d, J=8.0 Hz, 1H), 7.34 (d, J=2.8 Hz, 1H), 7.27 (dd, J=2.8, 9.1 Hz, 1H), 7.23 (d, J=7.2 Hz, 1H), 4.32 (s, 2H), 2.91 (s, 3H), 1.65 (d, J=7.2 Hz, 4H), 1.42 (d, J=6.8 Hz, 4H).

### Example Compound 1-13

### N-[4-(7-azabicyclo[2.2.1]heptan-7-yl)-2-(3-dimethylaminoprop-1-ynyl)phenyl]-4-oxo-5-(trifluoromethyl)-1H-quinoline-3-carboxamide

The preparation of the title compound is depicted in Scheme 1-12.

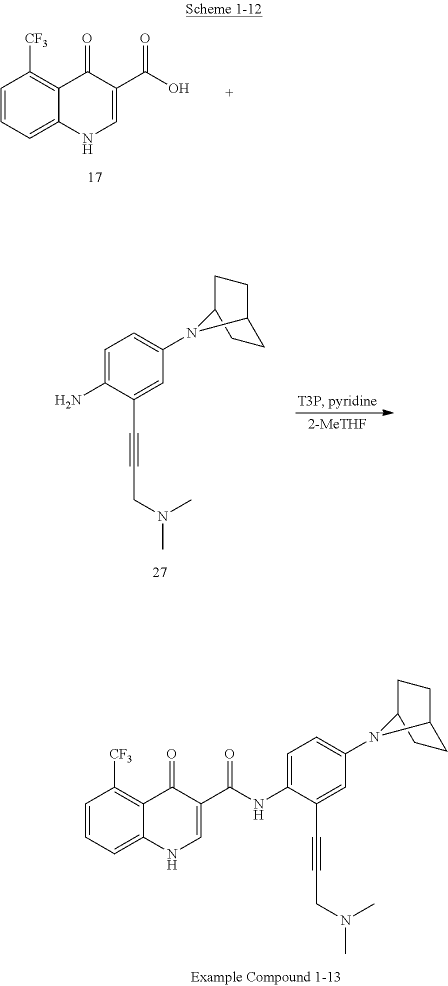

To a solution of 4-oxo-5-(trifluoromethyl)-1H-quinoline-3-carboxylic acid 17 (19 mg, 0.07 mmol) and 4-(7-azabicyclo[2.2.1]heptan-7-yl)-2-(3-dimethylaminoprop-1-ynyl)aniline 27 (20 mg, 0.07 mmol) in 2-methyltetrahydrofuran (190.9 μL) was added T3P (118 mg, 0.19 mmol) and pyridine (12 mg, 12 μL, 0.15 mmol). The reaction was heated at 100° C. for 30 min under microwave irradiation. The reaction was diluted with EtOAc and quenched with saturated aqueous NaHCO₃ (50 mL). The layers were separated, and the aqueous layer was extracted twice with EtOAc. The combined organics were washed once with water, dried over Na₂SO₄, filtered and concentrated. The residue was purified by reverse phase HPLC (0-99% CH₃CN/0.05% TFA) to give N-[4-(7-azabicyclo[2.2.1]heptan-7-yl)-2-(3-dimethylaminoprop-1-ynyl)phenyl]-4-oxo-5-(trifluoromethyl)-1H-quinoline-3-carboxamide (8 mg, 17% yield). LC/MS m/z 509.7 [M+H]⁺, retention time 1.06 min (RP-C₁₈, 10-99% CH₃CN/0.05% TFA over 3 min). ¹H NMR (400.0 MHz, DMSO-d₆) δ 13.23 (d, J=6.8 Hz, 1H), 12.40 (s, 1H), 10.31 (s, 1H), 8.96 (d, J=6.6 Hz, 1H), 8.40 (d, J=9.0 Hz, 1H), 8.08-8.06 (m, H), 8.07 (dd, J=1.5 Hz, 8.1 Hz, 1H), 8.00-7.95 (m, 2H), 7.15-7.09 (m, 2H), 4.49 (s, 2H), 4.29 (s, 2H), 2.94 (s, 6H), 1.67 (d, J=7.2 Hz, 4H), 1.44 (d, J=7.0 Hz, 4H).

### Example Compound 1-5

### Endo-N-[4-[(5S)-5-[tert-butyl(dimethyl)silyl]oxy-7-azabicyclo[2.2.1]heptan-7-yl]-2-(trifluoromethyl)phenyl]-4-oxo-5-(trifluoromethyl)-1H-quinoline-3-carboxamide

The preparation of the title compound is depicted in Scheme 1-13.

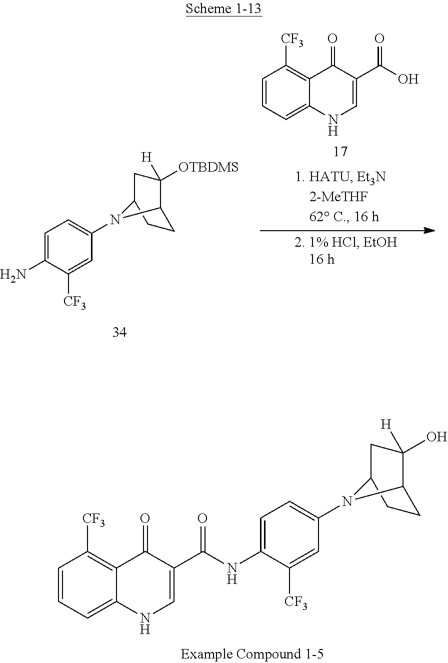

Preparation of endo-N-[4-[(5S)-5-[tert-butyl(dimethyl)silyl]oxy-7-azabicyclo[2.2.1]heptan-7-yl]-2-(trifluoromethyl)phenyl]-4-oxo-5-(trifluoromethyl)-1H-quinoline-3-carboxamide. To a solution of 4-oxo-5-(trifluoromethyl)-1H-quinoline-3-carboxylic acid 17 (148 mg, 0.58 mmol), O-(7-Azabenzotriazol-1-yl)-N,N,N′,N′-tetramethyluronium hexafluorophosphate (HATU) (306 mg, 0.81 mmol) in 2-methyltetrahydrofuran (2.2 mL) was added endo-4-[5-[tert-butyl(dimethyl)silyl]oxy-7-azabicyclo[2.2.1]heptan-7-yl]-2-(trifluoromethyl)aniline 34 (222 mg, 0.58 mmol) followed by triethylamine (146 mg, 201 μL, 1.44 mmol). The reaction mixture was heated at 62° C. for 16 h. The reaction mixture was allowed to cool to room temperature, and partitioned between 2-methyltetrahydrofuran/water, separated and the aqueous layer was extracted once more with 2-methyltetrahydrofuran, the organic layers were combined, dried over Na₂SO₄, filtered and concentrated to dryness. Purification by silica gel chromatography (0-30% ethyl acetate in dichloromethane) afforded endo-N-[4-[(5S)-5-[tert-butyl(dimethyl)silyl]oxy-7-azabicyclo[2.2.1]heptan-7-yl]-2-(trifluoromethyl)phenyl]-4-oxo-5-(trifluoromethyl)-1H-quinoline-3-carboxamide (285 mg, 79% yield). ¹H NMR (400.0 MHz, DMSO-d₆) δ 13.07 (s, 1H), 12.16 (s, 1H), 8.88 (s, 1H), 8.04 (dd, J=2.2, 7.3 Hz, 1H), 7.95-7.89 (m, 3H), 7.22 (dd, J=2.4, 8.9 Hz, 1H), 7.16 (d, J=2.6 Hz, 1H), 4.29 (m, 3H), 2.16-2.07 (m, 2H), 1.62-1.43 (m, 3H), 1.05-1.01 (m, 1H), 0.89 (s, 9H), 0.08 (d, J=1.4 Hz, 6H).

Preparation of endo-N-[4-[(5S)-5-hydroxy-7-azabicyclo[2.2.1]heptan-7-yl]-2-(trifluoromethyl)phenyl]-4-oxo-5-(trifluoromethyl)-1H-quinoline-3-carboxamide. Endo-N-[4-[(5S)-5-[tert-butyl(dimethyl)silyl]oxy-7-azabicyclo[2.2.1]heptan-7-yl]-2-(trifluoromethyl)phenyl]-4-oxo-5-(trifluoromethyl)-1H-quinoline-3-carboxamide (281 mg, 0.45 mmol) was dissolved in 1% HCl/Ethanol (2 mL of 1% w/w) solution and allowed to stir a room temperature for 16 h, resulting in a white precipitate. The reaction was diluted with diethyl ether and filtered. The collected solid was dissolved in ethyl acetate/saturated aqueous NaHCO₃ solution. The layers were separated and the aqueous layer was extracted once more with ethyl acetate. The organic layers were washed twice with water, dried over Na₂SO₄, filtered and concentrated to dryness to yield endo-N-[4-[(5S)-5-hydroxy-7-azabicyclo[2.2.1]heptan-7-yl]-2-(trifluoromethyl)phenyl]-4-oxo-5-(trifluoromethyl)-1H-quinoline-3-carboxamide (190 mg, 83%). ¹H NMR (400.0 MHz, DMSO-d₆) δ 13.07 (s, 1H), 12.15 (s, 1H), 8.88 (s, 1H), 8.04 (dd, J=2.2, 7.4 Hz, 1H), 7.95-7.88 (m, 3H), 7.19 (dd, J=2.4, 9.0 Hz, 1H), 7.12 (d, J=2.6 Hz, 1H), 5.00 (d, J=4.2 Hz, 1H), 4.25-4.13 (m, 1H), 4.21-4.19 (m, 1H), 4.16-4.13 (m, 1H), 2.15-2.08 (m, 2H), 1.61-1.55 (m, 1H), 1.47-1.44 (m, 2H) and 1.03 (dd, J=3.4, 12.3 Hz, 1H).

Analytical data for the compounds of Table 1 is shown below:

TABLE 2

| Example | LC/MS | LC/RT^{a} | |
|---|---|---|---|
| Compound No. | M + 1 | minutes | NMR |
| 1 | 496.0 | 1.48 | ¹H NMR (400.0 MHz, DMSO-d₆) δ 13.08 (s, 1H), |
| | | | 12.16 (s, 1H), 8.88 (s, 1H), 8.04 (dd, J = 2.1, 7.4 |
| | | | Hz, 1H), 7.95-7.88 (m, 3H), 7.22 (dd, 2.5, 8.9 Hz, |
| | | | 1H), 7.16 (d, J = 2.5 Hz, 1H), 4.33 (s, 2H), 1.67 (d, |
| | | | J = 6.9 Hz, 4H), 1.44 (d, J = 6.9 Hz, 4H). |
| 1-2 | 458.20 | 1.20 | ¹H NMR (400.0 MHz, DMSO-d₆) δ 12.89 (s, 1H), |
| | | | 12.43 (s, 1H), 8.79 (s, 1H), 8.00 (d, J = 8.9 Hz, |
| | | | 1H), 7.72-7.68 (m, 2H), 7.44 (dd, J = 2.9, 9.0 Hz, |
| | | | 1H), 7.22 (dd, J = 2.5, 9.0 Hz, 1H), 7.15 (d, J = |
| | | | 2.6 Hz, 1H), 4.33 (s, 2H), 3.91 (s, 3H), 1.67 (d, J = |
| | | | 6.9 Hz, 4H), 1.43 (d, J = 6.9 Hz, 4H). |
| 1-3 | 456.50 | 1.76 | ¹H NMR (400.0 MHz, DMSO-d₆) δ 12.94 (d, J = |
| | | | 6.1 Hz, 1H), 12.36 (s, 1H), 8.80 (d, J = 6.8 Hz, |
| | | | 1H), 8.13 (s, 1H), 7.98 (d, J = 8.9 Hz, 1H), 7.68-7.63 |
| | | | (m, 2H), 7.10 (d, J = 8.5 Hz, 1H), 7.01 (s, |
| | | | 1H), 4.28 (s, 2H), 2.48 (s, 3H), 2.02-2.00 (m, 2H), |
| | | | 1.86-1.77 (m, 5H), 1.45-1.42 (m, 1H), 1.31 (d, J = |
| | | | 11.1 Hz, 2H). |
| 1-4 | 458.50 | 1.22 | ¹H NMR (400.0 MHz, DMSO-d₆) δ 13.12 (d, J = |
| | | | 6.7 Hz, 1H), 12.50 (s, 1H), 8.78 (d, J = 6.8 Hz, |
| | | | 1H), 8.10 (d, J = 9.1 Hz, 1H), 7.78-7.72 (m, 3H), |
| | | | 7.65 (d, J = 7.7 Hz, 1H), 7.39 (d, J = 7.3 Hz, 1H), |
| | | | 7.33 (s, 1H), 5.20 (s, 2H), 4.47 (s, 2H), 1.74 (d, J = |
| | | | 6.7 Hz, 4H), 1.51 (d, J = 7.1 Hz, 4H). |
| 1-5 | 512.50 | 1.55 | ¹H NMR (400.0 MHz, DMSO-d₆) δ 13.07 (s, 1H), |
| | | | 12.15 (s, 1H), 8.88 (s, 1H), 8.04 (dd, J = 2.2, 7.4 |
| | | | Hz, 1H), 7.95-7.88 (m, 3H), 7.19 (dd, J = 2.4, 9.0 |
| | | | Hz, 1H), 7.12 (d, J = 2.6 Hz, 1H), 5.00 (d, J = 4.2 |
| | | | Hz, 1H), 4.25-4.13 (m, 1H), 4.21-4.19 (m, 1H), |
| | | | 4.16-4.13 (m, 1H), 2.15-2.08 (m, 2H), 1.61-1.55 |
| | | | (m, 1H), 1.47-1.44 (m, 2H) and 1.03 (dd, J = |
| | | | 3.4, 12.3 Hz, 1H). |
| 1-6 | 399.30 | 1.47 | ¹H NMR (400.0 MHz, DMSO-d₆) δ 12.77 (s, 1H), |
| | | | 12.75 (s, 1H), 8.77 (s, 1H), 8.11 (d, J = 9.1 Hz, |
| | | | 1H), 7.64-7.60 (m, 1H), 7.55 (d, J = 8.0 Hz, 1H), |
| | | | 7.34 (d, J = 2.8 Hz, 1H), 7.27 (dd, J = 2.8, 9.1 Hz, |
| | | | 1H), 7.23 (d, J = 7.2 Hz, 1H), 4.32 (s, 2H), 2.91 (s, |
| | | | 3H), 1.65 (d, J = 7.2 Hz, 4H), 1.42 (d, J = 6.8 Hz, |
| | | | 4H). |
| 1-7 | 453.0 | 1.62 | ¹H NMR (400.0 MHz, DMSO-d₆) δ 13.28 (d, J = |
| | | | 6.4 Hz, 1H), 12.07 (s, 1H), 8.95 (d, J = 6.5 Hz, |
| | | | 1H), 8.69 (s, 1H), 8.16 (dd, J = 1.5, 8.7 Hz, 1H), |
| | | | 8.01 (d, J = 8.8 Hz, 1H), 7.91 (d, J = 8.7 Hz, 1H), |
| | | | 7.28 (d, J = 7.8 Hz, 1H), 7.22 (s, 1H), 4.38 (s, 2H), |
| | | | 1.69 (d, J = 6.4 Hz, 4H), 1.46 (d, J = 6.9 Hz, 4H). |
| 1-8 | 512.10 | 1.35 | ¹H NMR (400.0 MHz, DMSO-d₆) δ 13.07 (s, 1H), |
| | | | 12.13 (s, 1H), 8.88 (s, 1H), 8.05-8.02 (m, 1H), |
| | | | 7.95-7.86 (m, 3H), 7.18 (d, J = 9.0 Hz, 1H), 7.12 |
| | | | (d, J = 2.5 Hz, 1H), 4.74 (d, J = 5.2 Hz, 1H), 4.33 |
| | | | (m, 1H), 4.11 (m, 1H), 3.77 (m, 1H), 1.82 (dd, J = |
| | | | 7.3, 12.5 Hz, 1H), 1.56-1.48 (m, 3H), 1.25 (m, |
| | | | 2H). |
| 1-9 | 442.10 | 1.20 | ¹H NMR (400.0 MHz, DMSO-d₆) δ 12.84 (s, |
| | | | 1H), 12.43 (s, 1H), 8.81 (s, 1H), 8.12 (s, 1H), 8.00 |
| | | | (d, J = 8.9 Hz, 1H), 7.64 (m, 2H), 7.21 (dd, J = |
| | | | 2.5, 9.0 Hz, 1H), 7.15 (d, J = 2.6 Hz, 1H), 4.32 (s, |
| | | | 2H), 2.47 (s, 3H), 1.67 (d, J = 7.4 Hz, 4H), 1.43 (d, |
| | | | J = 6.9 Hz, 4H). |
| 1-10 | 442.10 | 1.40 | ¹H NMR (400.0 MHz, DMSO-d₆) δ 12.68 (s, 1H), |
| | | | 12.41 (s, 1H), 8.75 (s, 1H), 7.94 (d, J = 8.9 Hz, |
| | | | 1H), 7.63-7.60 (m, 1H), 7.54 (d, J = 7.8 Hz, 1H), |
| | | | 7.23-7.20 (m, 2H), 7.15 (d, J = 2.7 Hz, 1H), 4.33 |
| | | | (s, 2H), 2.89 (s, 3H), 1.67 (d, J = 6.7 Hz, 4H), 1.44 |
| | | | (d, J = 6.9 Hz, 4H). |
| 1-11 | 444.0 | 1.30 | ¹H NMR (400.0 MHz, DMSO-d₆) δ 13.55 (s, 1H), |
| | | | 13.31 (d, J = 7.2 Hz, 1H), 11.58 (s, 1H), 8.86 (d, J = |
| | | | 6.9 Hz, 1H), 8.01 (d, J = 9.0 Hz, 1H), 7.66 (t, J = |
| | | | 8.2 Hz, 1H), 7.29 (d, J = 8.8 Hz, 1H), 7.23 (s, |
| | | | 1H), 7.17 (d, J = 7.7 Hz, 1H), 6.80 (d, J = 7.5 Hz, |
| | | | 1H), 4.39 (s, 2H), 1.69 (d, J = 7.3 Hz, 4H), 1.46 (d, |
| | | | J = 7.0 Hz, 4H). |
| 1-12 | 510.5 | 1.95 | ¹H NMR (400.0 MHz, DMSO-d₆) δ 13.16 (d, J = |
| | | | 5.7 Hz, 1H), 12.07 (s, 1H), 8.87 (d, J = 6.6 Hz, |
| | | | 1H), 8.05 (dd, J = 2.1, 7.3 Hz, 1H), 7.96-7.92 (m, |
| | | | 2H), 7.87 (d, J = 9.0 Hz, 1H), 7.09 (d, J = 9.1 Hz, |
| | | | 1H), 7.00 (s, 1H), 4.28 (s, 2H), 2.02-2.00 (m, 2H), |
| | | | 1.88-1.73 (m, 5H), 1.45-1.42 (m, 1H), 1.31 (d, J = |
| | | | 11.6 Hz, 2H). |
| 1-13 | 509.7 | 1.06 | ¹H NMR (400.0 MHz, DMSO-d₆) δ 13.23 (d, J = |
| | | | 6.8 Hz, 1H), 12.40 (s, 1H), 10.31 (s, 1H), 8.96 (d, |
| | | | J = 6.6 Hz, 1H), 8.40 (d, J = 9.0 Hz, 1H), 8.08-8.06 |
| | | | (m, H), 8.07 (dd, J = 1.5 Hz, 8.1 Hz, 1H), 8.00-7.95 |
| | | | (m, 2H), 7.15-7.09 (m, 2H), 4.49 (s, 2H), |
| | | | 4.29 (s, 2H), 2.94 (s, 6H), 1.67 (d, J = 7.2 Hz, 4H), |
| | | | 1.44 (d, J= 7.0 Hz, 4H). |
| 1-14 | 455.7 | 1.04 | ¹H NMR (400.0 MHz, DMSO-d₆) δ 12.63 (s, 2H), |
| | | | 8.75 (s, 1H), 8.38 (d, J = 8.8 Hz, 1H), 7.62-7.58 |
| | | | (m, 1H), 7.52 (d, J = 8.1 Hz, 1H), 7.21 (d, J = 7.2 |
| | | | Hz, 1H), 6.96-6.93 (m, 2H), 4.24 (s, 2H), 3.70 (s, |
| | | | 2H), 2.92 (s, 3H), 2.28 (s, 6H), 1.65 (d, J = 7.0 Hz, |
| | | | 4H), 1.39 (d, J = 6.8 Hz, 4H). |

^{a}Retention Time

### II.B. Compounds of Formula II

### II.B.1. Embodiments of the Compounds of Formula II

In one aspect the invention includes a pharmaceutical composition comprising a Compound of Formula II

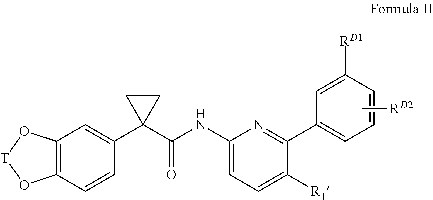

or pharmaceutically acceptable salts thereof, wherein:

T is —CH₂—, —CH₂CH₂—, —CF₂—, —C(CH₃)₂—, or —C(O)—;

R₁′ is H, C₁₋₆ aliphatic, halo, CF₃, CHF₂, O(C₁₋₆ aliphatic); and

R^{D1} or R^{D2} is Z^{D}R₉
wherein:
Z^{D} is a bond, CONH, SO₂NH, SO₂N(C₁₋₆ alkyl), CH₂NHSO₂, CH₂N(CH₃)SO₂, CH₂NHCO, COO, SO₂, or CO; and
R₉ is H, C₁₋₆ aliphatic, or aryl.

### II.B.2. Compound 2

In another embodiment, the compound of Formula II is Compound 2, depicted below, which is also known by its chemical name 3-(6-(1-(2,2-difluorobenzo[d][1,3]dioxol-5-yl)cyclopropanecarboxamido)-3-methylpyridin-2-yl)benzoic acid.

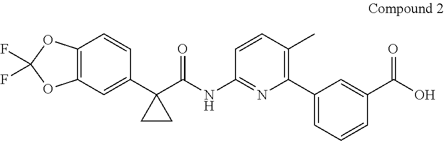

### II.B.3. Overview of the Synthesis of Compound 2

Compounds of Formula II, as exemplified by Compound 2, can be prepared by coupling an acid chloride moiety with an amine moiety according to following Schemes 2-1a to 2-3.

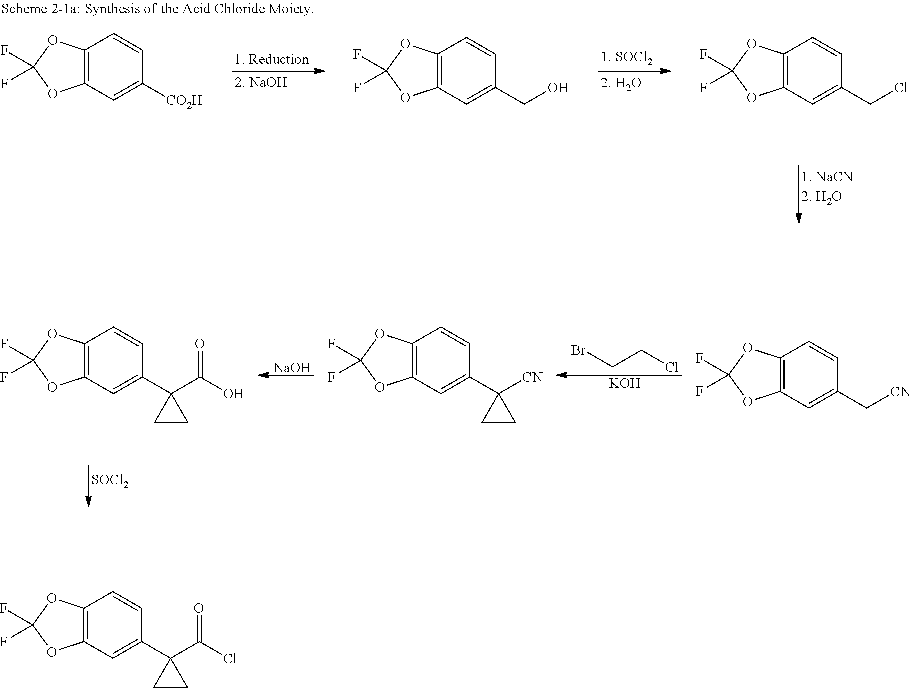

Scheme 2-1a depicts the preparation of 1-(2,2-difluorobenzo[d][1,3]dioxol-5-yl)cyclopropanecarbonyl chloride, which is used in Scheme 3 to make the amide linkage of Compound 2.

The starting material, 2,2-difluorobenzo[d][1,3]dioxole-5-carboxylic acid, is commercially available from Saltigo (an affiliate of the Lanxess Corporation). Reduction of the carboxylic acid moiety in 2,2-difluorobenzo[d][1,3]dioxole-5-carboxylic acid to the primary alcohol, followed by conversion to the corresponding chloride using thionyl chloride (SOCl₂), provides 5-(chloromethyl)-2,2-difluorobenzo[d][1,3]dioxole, which is subsequently converted to 2-(2,2-difluorobenzo[d][1,3]dioxol-5-yl)acetonitrile using sodium cyanide. Treatment of 2-(2,2-difluorobenzo[d][1,3]dioxol-5-yl)acetonitrile with base and 1-bromo-2-chloroethane provides 1-(2,2-difluorobenzo[d][1,3]dioxol-5-yl)cyclopropanecarbonitrile. The nitrile moiety in 1-(2,2-difluorobenzo[d][1,3]dioxol-5-yl)cyclopropanecarbonitrile is converted to a carboxylic acid using base to give 1-(2,2-difluorobenzo[d][1,3]dioxol-5-yl)cyclopropanecarboxylic acid, which is converted to the desired acid chloride using thionyl chloride.

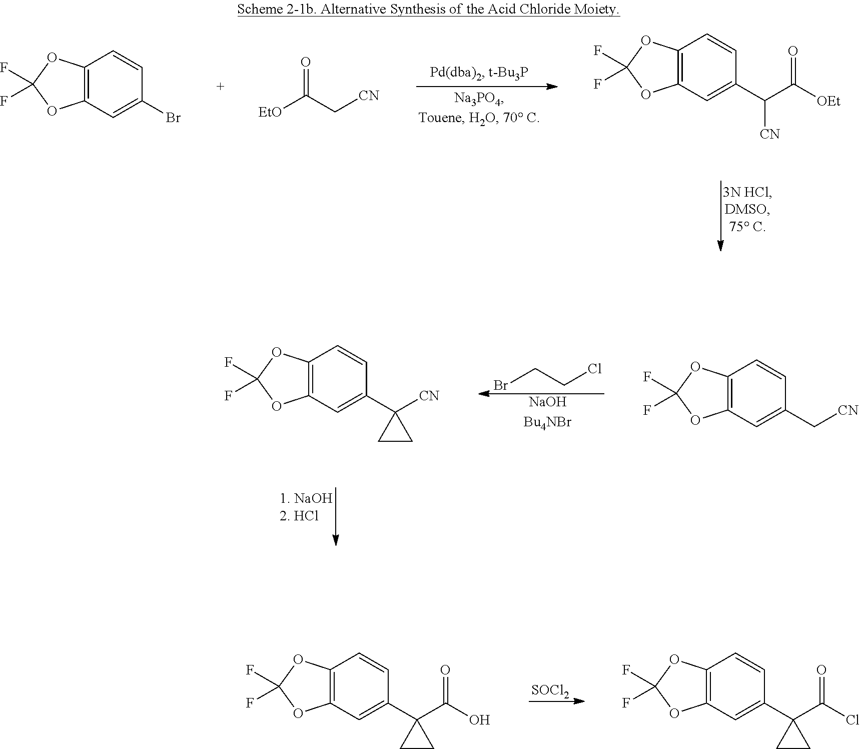

Scheme 2-1b provides an alternative synthesis of the requisite acid chloride. The compound 5-bromomethyl-2,2-difluoro-1,3-benzodioxole is coupled with ethyl cyanoacetate in the presence of a palladium catalyst to form the corresponding alpha cyano ethyl ester. Saponification of the ester moiety to the carboxylic acid gives the cyanoethyl compound. Alkylation of the cyanoethyl compound with 1-bromo-2-chloro ethane in the presence of base gives the cyanocyclopropyl compound. Treatment of the cyanocyclopropyl compound with base gives the carboxylate salt, which is converted to the carboxylic acid by treatment with acid. Conversion of the carboxylic acid to the acid chloride is then accomplished using a chlorinating agent such as thionyl chloride or the like.

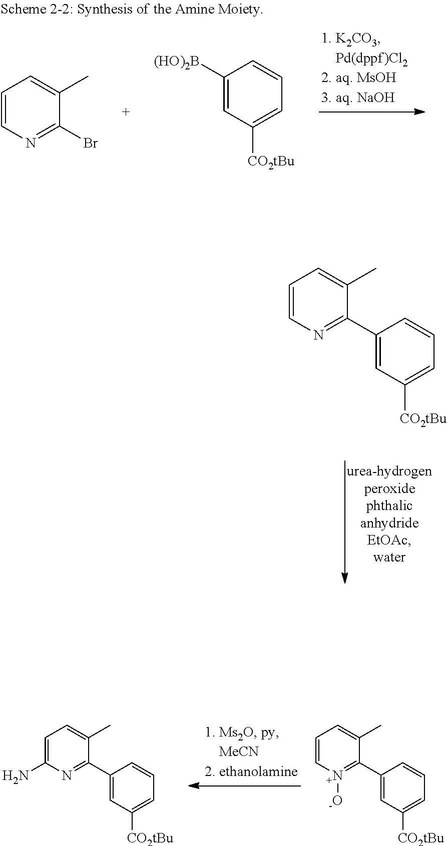

Scheme 2-2 depicts the preparation of the requisite tert-butyl 3-(6-amino-3-methylpyridin-2-yl)benzoate, which is coupled with 1-(2,2-difluorobenzo[d][1,3]dioxol-5-yl)cyclopropanecarbonyl chloride in Scheme 3 to give Compound 2. Palladium-catalyzed coupling of 2-bromo-3-methylpyridine with 3-(tert-butoxycarbonyl)phenylboronic acid gives tert-butyl 3-(3-methylpyridin-2-yl)benzoate, which is subsequently converted to the desired compound.

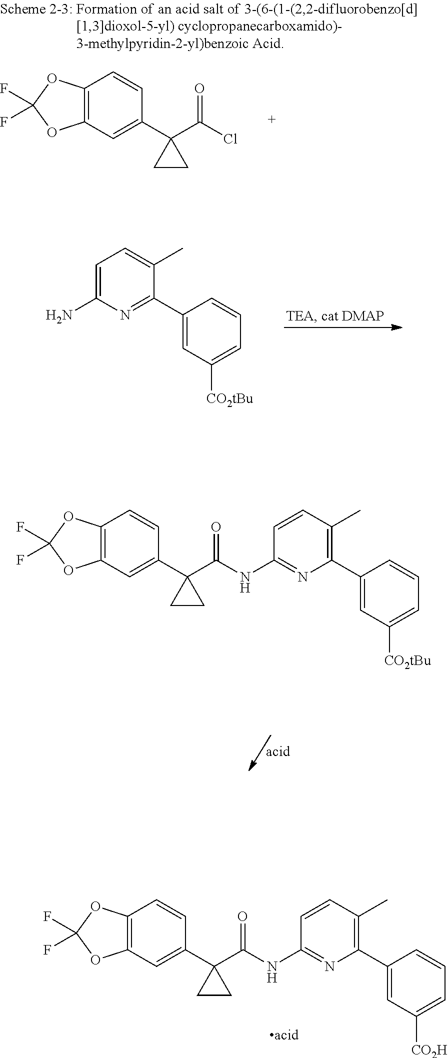

Scheme 2-3 depicts the coupling of 1-(2,2-difluorobenzo[d][1,3]dioxol-5-yl)cyclopropanecarbonyl chloride with tert-butyl 3-(6-amino-3-methylpyridin-2-yl)benzoate using triethyl amine and 4-dimethylaminopyridine to initially provide the tert-butyl ester of Compound 2. Treatment of the tert-butyl ester with an acid such as HCl, gives the HCl salt of Compound 2, which is typically a crystalline solid.

### II.B.4. Examples: Synthesis of Compound 2

Vitride® (sodium bis(2-methoxyethoxy)aluminum hydride [or NaAlH₂(OCH₂CH₂OCH₃)₂], 65 wgt % solution in toluene) was purchased from Aldrich Chemicals. 2,2-Difluoro-1,3-benzodioxole-5-carboxylic acid was purchased from Saltigo (an affiliate of the Lanxess Corporation).

### Example 2a

### (2,2-Difluoro-1,3-benzodioxol-5-yl)-methanol

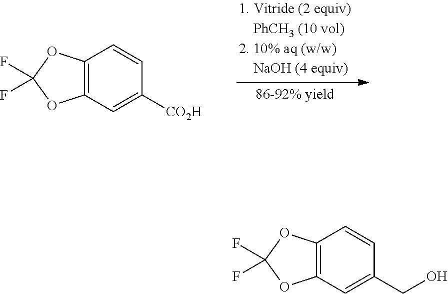

Commercially available 2,2-difluoro-1,3-benzodioxole-5-carboxylic acid (1.0 eq) was slurried in toluene (10 vol). Vitride® (2 eq) was added via addition funnel at a rate to maintain the temperature at 15-25° C. At the end of the addition, the temperature was increased to 40° C. for 2 hours (h), then 10% (w/w) aqueous (aq) NaOH (4.0 eq) was carefully added via addition funnel, maintaining the temperature at 40-50° C. After stirring for an additional 30 minutes (min), the layers were allowed to separate at 40° C. The organic phase was cooled to 20° C., then washed with water (2×1.5 vol), dried (Na₂SO₄), filtered, and concentrated to afford crude (2,2-difluoro-1,3-benzodioxol-5-yl)-methanol that was used directly in the next step.

### Example 2b

### 5-Chloromethyl-2,2-difluoro-1,3-benzodioxole

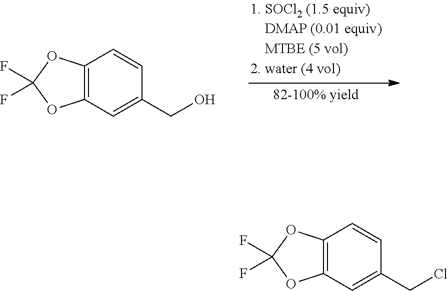

(2,2-Difluoro-1,3-benzodioxol-5-yl)-methanol (1.0 eq) was dissolved in MTBE (5 vol). A catalytic amount of 4-(N,N-dimethyl)aminopyridine (DMAP) (1 mol %) was added and SOCl₂ (1.2 eq) was added via addition funnel. The SOCl₂ was added at a rate to maintain the temperature in the reactor at 15-25° C. The temperature was increased to 30° C. for 1 h, and then was cooled to 20° C. Water (4 vol) was added via addition funnel while maintaining the temperature at less than 30° C. After stirring for an additional 30 min, the layers were allowed to separate. The organic layer was stirred and 10% (w/v) aq NaOH (4.4 vol) was added. After stirring for 15 to 20 min, the layers were allowed to separate. The organic phase was then dried (Na₂SO₄), filtered, and concentrated to afford crude 5-chloromethyl-2,2-difluoro-1,3-benzodioxole that was used directly in the next step.

### Example 2c

### (2,2-Difluoro-1,3-benzodioxol-5-yl)-acetonitrile

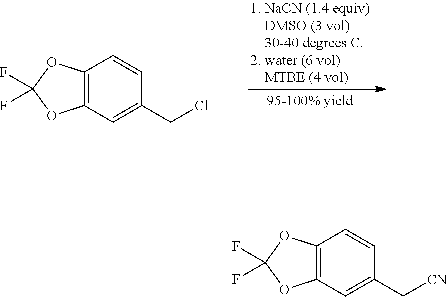

A solution of 5-chloromethyl-2,2-difluoro-1,3-benzodioxole (1 eq) in DMSO (1.25 vol) was added to a slurry of NaCN (1.4 eq) in DMSO (3 vol), while maintaining the temperature between 30-40° C. The mixture was stirred for 1 h, and then water (6 vol) was added, followed by methyl tert-butyl ether (MTBE) (4 vol). After stirring for 30 min, the layers were separated. The aqueous layer was extracted with MTBE (1.8 vol). The combined organic layers were washed with water (1.8 vol), dried (Na₂SO₄), filtered, and concentrated to afford crude (2,2-difluoro-1,3-benzodioxol-5-yl)-acetonitrile (95%) that was used directly in the next step. ¹H NMR (500 MHz, DMSO) δ 7.44 (br s, 1H), 7.43 (d, J=8.4 Hz, 1H), 7.22 (dd, J=8.2, 1.8 Hz, 1H), 4.07 (s, 2H).

### Example 2d

### Alternate Synthesis of (2,2-difluoro-1,3-benzodioxol-5-yl)-1-ethylacetate-acetonitrile

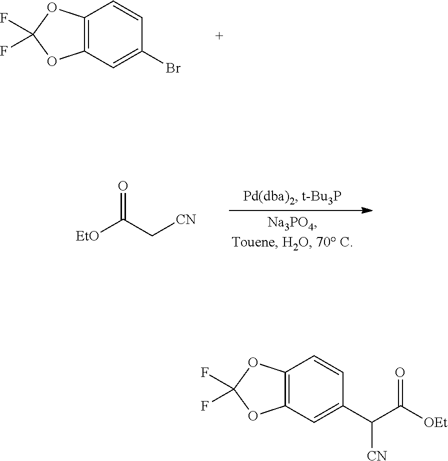

A reactor was purged with nitrogen and charged with toluene (900 mL). The solvent was degassed via nitrogen sparge for no less than 16 hours. To the reactor was then charged Na₃PO₄ (155.7 g, 949.5 mmol), followed by bis(dibenzylideneacetone) palladium (0) (7.28 g, 12.66 mmol). A 10% w/w solution of tert-butylphosphine in hexanes (51.23 g, 25.32 mmol) was charged over 10 minutes at 23° C. from a nitrogen purged addition funnel. The mixture was allowed to stir for 50 minutes, at which time 5-bromo-2,2-difluoro-1,3-benzodioxole (75 g, 316.5 mmol) was added over 1 minute. After stirring for an additional 50 minutes, the mixture was charged with ethyl cyanoacetate (71.6 g, 633.0 mmol) over 5 minutes, followed by water (4.5 mL) in one portion. The mixture was heated to 70° C. over 40 minutes and analyzed by HPLC every 1 to 2 hours for the percent conversion of the reactant to the product. After complete conversion was observed (typically 100% conversion after 5 to 8 hours), the mixture was cooled to 20 to 25° C. and filtered through a celite pad. The celite pad was rinsed with toluene (2×450 mL), and the combined organics were concentrated to 300 mL under vacuum at 60 to 65° C. The concentrate was charged with DMSO (225 mL) and concentrated under vacuum at 70 to 80° C. until active distillation of the solvent ceased. The solution was cooled to 20 to 25° C. and diluted to 900 mL with DMSO in preparation for Step 2. ¹H NMR (500 MHz, CDCl₃) δ 7.16-7.10 (m, 2H), 7.03 (d, J=8.2 Hz, 1H), 4.63 (s, 1H), 4.19 (m, 2H), 1.23 (t, J=7.1 Hz, 3H).

### Example 2e

### Alternate Synthesis of (2,2-difluoro-1,3-benzodioxol-5-yl)-acetonitrile

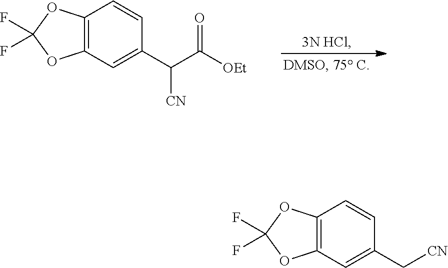

The DMSO solution of (2,2-difluoro-1,3-benzodioxol-5-yl)-1-ethylacetate-acetonitrile from above was charged with 3 N HCl (617.3 mL, 1.85 mol) over 20 minutes while maintaining an internal temperature less than 40° C. The mixture was then heated to 75° C. over 1 hour and analyzed by HPLC every 1 to 2 hour for percent conversion. When a conversion of greater than 99% was observed (typically after 5 to 6 hours), the reaction was cooled to 20 to 25° C. and extracted with MTBE (2×525 mL), with sufficient time to allow for complete phase separation during the extractions. The combined organic extracts were washed with 5% NaCl (2×375 mL). The solution was then transferred to equipment appropriate for a 1.5 to 2.5 Ton vacuum distillation that was equipped with a cooled receiver flask. The solution was concentrated under vacuum at less than 60° C. to remove the solvents. (2,2-Difluoro-1,3-benzodioxol-5-yl)-acetonitrile was then distilled from the resulting oil at 125 to 130° C. (oven temperature) and 1.5 to 2.0 Torr. (2,2-Difluoro-1,3-benzodioxol-5-yl)-acetonitrile was isolated as a clear oil in 66% yield from 5-bromo-2,2-difluoro-1,3-benzodioxole (2 steps) and with an HPLC purity of 91.5% AUC (corresponds to a w/w assay of 95%). ¹H NMR (500 MHz, DMSO) δ 7.44 (br s, 1H), 7.43 (d, J=8.4 Hz, 1H), 7.22 (dd, J=8.2, 1.8 Hz, 1H), 4.07 (s, 2H).

### Example 2f

### (2,2-Difluoro-1,3-benzodioxol-5-yl)-cyclopropanecarbonitrile

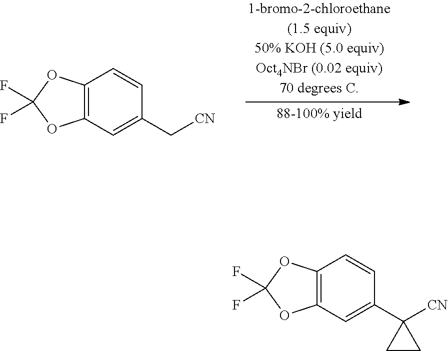

A mixture of (2,2-difluoro-1,3-benzodioxol-5-yl)-acetonitrile (1.0 eq), 50 wt % aqueous KOH (5.0 eq) 1-bromo-2-chloroethane (1.5 eq), and Oct₄NBr (0.02 eq) was heated at 70° C. for 1 h. The reaction mixture was cooled, then worked up with MTBE and water. The organic phase was washed with water and brine. The solvent was removed to afford (2,2-difluoro-1,3-benzodioxol-5-yl)-cyclopropanecarbonitrile. ¹H NMR (500 MHz, DMSO) δ 7.43 (d, J=8.4 Hz, 1H), 7.40 (d, J=1.9 Hz, 1H), 7.30 (dd, J=8.4, 1.9 Hz, 1H), 1.75 (m, 2H), 1.53 (m, 2H).

### Example 2g

### 1-(2,2-Difluoro-1,3-benzodioxol-5-yl)-cyclopropanecarboxylic acid

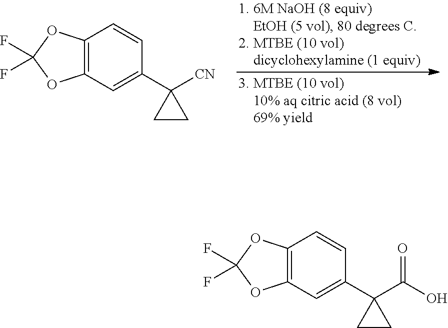

(2,2-Difluoro-1,3-benzodioxol-5-yl)-cyclopropanecarbonitrile was hydrolyzed using 6 M NaOH (8 equiv) in ethanol (5 vol) at 80° C. overnight. The mixture was cooled to room temperature and the ethanol was evaporated under vacuum. The residue was taken up in water and MTBE, 1 M HCl was added, and the layers were separated. The MTBE layer was then treated with dicyclohexylamine (DCHA) (0.97 equiv). The slurry was cooled to 0° C., filtered and washed with heptane to give the corresponding DCHA salt. The salt was taken into MTBE and 10% citric acid and stirred until all the solids had dissolved. The layers were separated and the MTBE layer was washed with water and brine. A solvent swap to heptane followed by filtration gave 1-(2,2-difluoro-1,3-benzodioxol-5-yl)-cyclopropanecarboxylic acid after drying in a vacuum oven at 50° C. overnight. ESI-MS m/z calc. 242.04. found 241.58 (M+1)⁺; ¹H NMR (500 MHz, DMSO) δ 12.40 (s, 1H), 7.40 (d, J=1.6 Hz, 1H), 7.30 (d, J=8.3 Hz, 1H), 7.17 (dd, J=8.3, 1.7 Hz, 1H), 1.46 (m, 2H), 1.17 (m, 2H).

### Example 2h

### 1-(2,2-Difluoro-1,3-benzodioxol-5-yl)-cyclopropanecarbonyl chloride

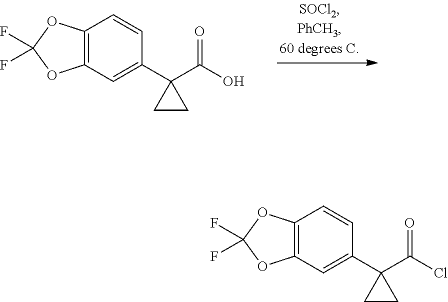

1-(2,2-Difluoro-1,3-benzodioxol-5-yl)-cyclopropanecarboxylic acid (1.2 eq) is slurried in toluene (2.5 vol) and the mixture was heated to 60° C. SOCl₂ (1.4 eq) was added via addition funnel. The toluene and SOCl₂ were distilled from the reaction mixture after 30 minutes. Additional toluene (2.5 vol) was added and the resulting mixture was distilled again, leaving the product acid chloride as an oil, which was used without further purification.

### Example 2i

### tert-Butyl-3-(3-methylpyridin-2-yl)benzoate

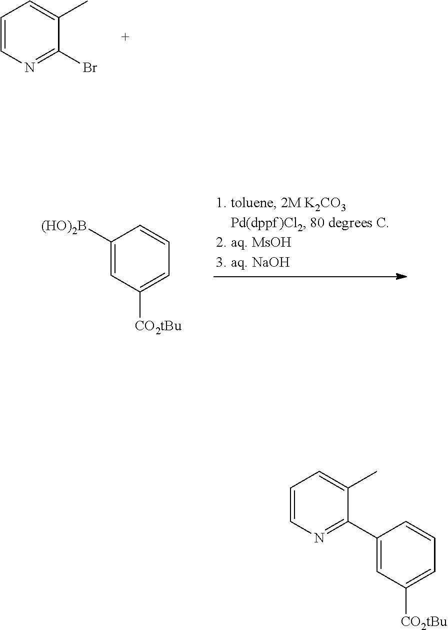

2-Bromo-3-methylpyridine (1.0 eq) was dissolved in toluene (12 vol). K₂CO₃ (4.8 eq) was added, followed by water (3.5 vol). The resulting mixture was heated to 65° C. under a stream of N₂ for 1 hour. 3-(t-Butoxycarbonyl)phenylboronic acid (1.05 eq) and Pd(dppf)Cl₂—CH₂Cl₂ (0.015 eq) were then added and the mixture was heated to 80° C. After 2 hours, the heat was turned off, water was added (3.5 vol), and the layers were allowed to separate. The organic phase was then washed with water (3.5 vol) and extracted with 10% aqueous methanesulfonic acid (2 eq MsOH, 7.7 vol). The aqueous phase was made basic with 50% aqueous NaOH (2 eq) and extracted with EtOAc (8 vol). The organic layer was concentrated to afford crude tert-butyl-3-(3-methylpyridin-2-yl)benzoate (82%) that was used directly in the next step.

### Example 2j

### 2-(3-(tert-Butoxycarbonyl)phenyl)-3-methylpyridine-1-oxide

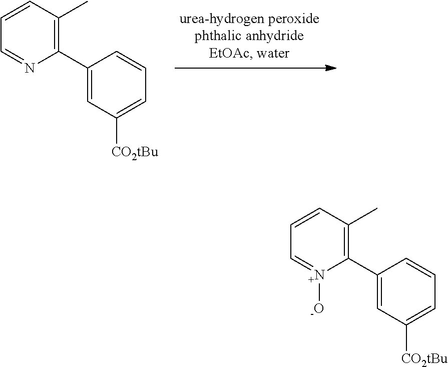

tert-Butyl-3-(3-methylpyridin-2-yl)benzoate (1.0 eq) was dissolved in EtOAc (6 vol). Water (0.3 vol) was added, followed by urea-hydrogen peroxide (3 eq). Phthalic anhydride (3 eq) was then added portionwise to the mixture as a solid at a rate to maintain the temperature in the reactor below 45° C. After completion of the phthalic anhydride addition, the mixture was heated to 45° C. After stirring for an additional 4 hours, the heat was turned off. 10% w/w aqueous Na₂SO₃ (1.5 eq) was added via addition funnel. After completion of Na₂SO₃ addition, the mixture was stirred for an additional 30 min and the layers separated. The organic layer was stirred and 10% wt/wt aqueous. Na₂CO₃ (2 eq) was added. After stirring for 30 minutes, the layers were allowed to separate. The organic phase was washed 13% w/v aq NaCl. The organic phase was then filtered and concentrated to afford crude 2-(3-(tert-butoxycarbonyl)phenyl)-3-methylpyridine-1-oxide (95%) that was used directly in the next step.

### Example 2k

### tert-Butyl-3-(6-amino-3-methylpyridin-2-yl)benzoate

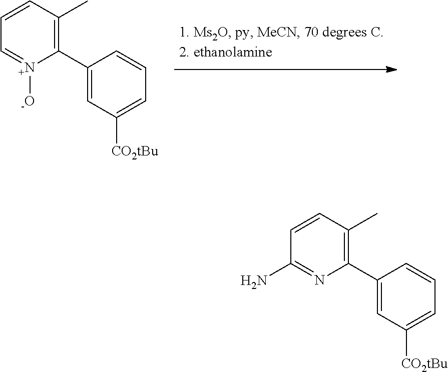

A solution of 2-(3-(tert-butoxycarbonyl)phenyl)-3-methylpyridine-1-oxide (1 eq) and pyridine (4 eq) in acetonitrile (8 vol) was heated to 70° C. A solution of methanesulfonic anhydride (1.5 eq) in MeCN (2 vol) was added over 50 min via addition funnel while maintaining the temperature at less than 75° C. The mixture was stirred for an additional 0.5 hours after complete addition. The mixture was then allowed to cool to ambient temperature. Ethanolamine (10 eq) was added via addition funnel. After stirring for 2 hours, water (6 vol) was added and the mixture was cooled to 10° C. After stirring for 3 hours, the solid was collected by filtration and washed with water (3 vol), 2:1 acetonitrile/water (3 vol), and acetonitrile (2×1.5 vol). The solid was dried to constant weight (<1% difference) in a vacuum oven at 50° C. with a slight N₂ bleed to afford tert-butyl-3-(6-amino-3-methylpyridin-2-yl)benzoate as a red-yellow solid (53% yield).

### Example 2l

### 3-(6-(1-(2,2-Difluorobenzo[d][1,3]dioxol-5-yl)-cyclopropanecarboxamido)-3-methylpyridin-2-yl)-t-butylbenzoate

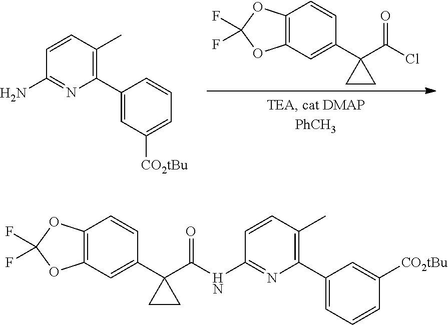

The crude acid chloride described above was dissolved in toluene (2.5 vol based on acid chloride) and added via addition funnel to a mixture of tert-butyl-3-(6-amino-3-methylpyridin-2-yl)benzoate (1 eq), DMAP, (0.02 eq), and triethylamine (3.0 eq) in toluene (4 vol based on tert-butyl-3-(6-amino-3-methylpyridin-2-yl)benzoate). After 2 hours, water (4 vol based on tert-butyl-3-(6-amino-3-methylpyridin-2-yl)benzoate) was added to the reaction mixture. After stirring for 30 minutes, the layers were separated. The organic phase was then filtered and concentrated to afford a thick oil of 3-(6-(1-(2,2-difluorobenzo[d][1,3]dioxol-5-yl)cyclopropanecarboxamido)-3-methylpyridin-2-yl)-t-butylbenzoate (quantitative crude yield). Acetonitrile (3 vol based on crude product) was added and distilled until crystallization occurs. Water (2 vol based on crude product) was added and the mixture stirred for 2 h. The solid was collected by filtration, washed with 1:1 (by volume) acetonitrile/water (2×1 volumes based on crude product), and partially dried on the filter under vacuum. The solid was dried to a constant weight (<1% difference) in a vacuum oven at 60° C. with a slight N₂ bleed to afford 3-(6-(1-(2,2-difluorobenzo[d][1,3]dioxol-5-yl)cyclopropanecarboxamido)-3-methylpyridin-2-yl)-t-butylbenzoate as a brown solid.

### Example 2m

### 3-(6-(1-(2,2-Difluorobenzo[d][1,3]dioxol-5-yl)cyclopropanecarboxamido)-3-methylpyridin-2-yl)benzoic acid.HCl salt

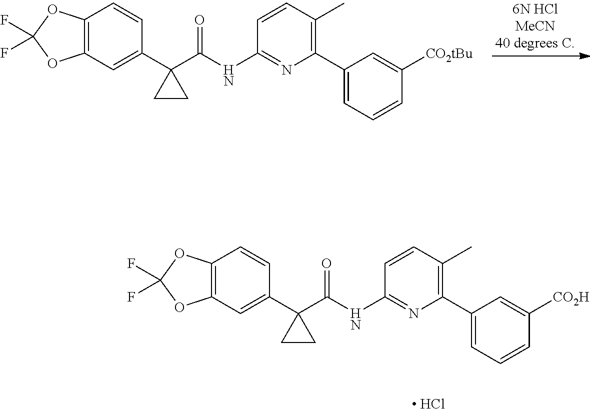

To a slurry of 3-(6-(1-(2,2-difluorobenzo[d][1,3]dioxol-5-yl)cyclopropanecarboxamido)-3-methylpyridin-2-yl)-t-butylbenzoate (1.0 eq) in MeCN (3.0 vol) was added water (0.83 vol) followed by concentrated aqueous HCl (0.83 vol). The mixture was heated to 45±5° C. After stirring for 24 to 48 h, the reaction was complete, and the mixture was allowed to cool to ambient temperature. Water (1.33 vol) was added and the mixture stirred. The solid was collected by filtration, washed with water (2×0.3 vol), and partially dried on the filter under vacuum. The solid was dried to a constant weight (<1% difference) in a vacuum oven at 60° C. with a slight N₂ bleed to afford 3-(6-(1-(2,2-difluorobenzo[d][1,3]dioxol-5-yl)cyclopropanecarboxamido)-3-methylpyridin-2-yl)benzoic acid.HCl as an off-white solid.

Table 2-1 below recites physical data for Compound 2.

TABLE 2-1

| | LC/MS | LC/RT | |
|---|---|---|---|
| Compound | M + 1 | minutes | NMR |
| Compound | 453.3 | 1.93 | ¹HNMR (400 MHz, DMSO-d6) 9.14 (s, |
| 2 | | | 1H), 7.99-7.93 (m, 3H), 7.80-7.78 |
| | | | (m, 1H), 7.74-7.72 (m, 1H), 7.60-7.55 |
| | | | (m, 2H), 7.41-7.33 (m, 2H), 2.24 |
| | | | (s, 3H), 1.53-1.51 (m, 2H), 1.19-1.17 |
| | | | (m, 2H). |

### II.C. Compounds of Formula III

### II.C.1. Embodiments of Compounds of Formula III

In one aspect the invention includes a pharmaceutical composition comprising a Compound of Formula III

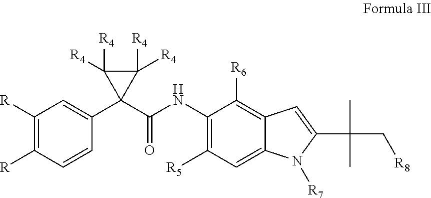

or pharmaceutically acceptable salts thereof, wherein:
R is H, OH, OCH₃ or two R taken together form —OCH₂O— or —OCF₂O—;
R₄ is H or alkyl;
R₅ is H or
R₆ is H or CN;
R₇ is H, —CH₂CH(OH)CH₂OH, —CH₂CH₂N⁺(CH₃)₃, or —CH₂CH₂OH;
R₈ is H, OH, —CH₂CH(OH)CH₂OH, —CH₂OH, or R₇ and R₈ taken together form a five membered ring.

### II.C.2. Compound 3

In another embodiment, the compound of Formula III is Compound 3, which is known by its chemical name (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide.

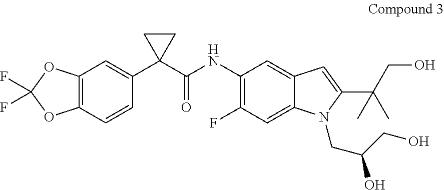

### II.C.3. Overview of the Synthesis of Compound 3

Compound 3 can be prepared by coupling an acid chloride moiety with an amine moiety according to the schemes below.

II.C.3.a. Synthesis of the Acid Moiety of Compound 3

The acid moiety of Compound 3 can be synthesized as the acid chloride,

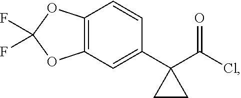

according to Scheme 2-1a, Scheme 2-1b and Examples 2a-2h.
II.C.3.b. Synthesis of the Amine Moiety of Compound 3

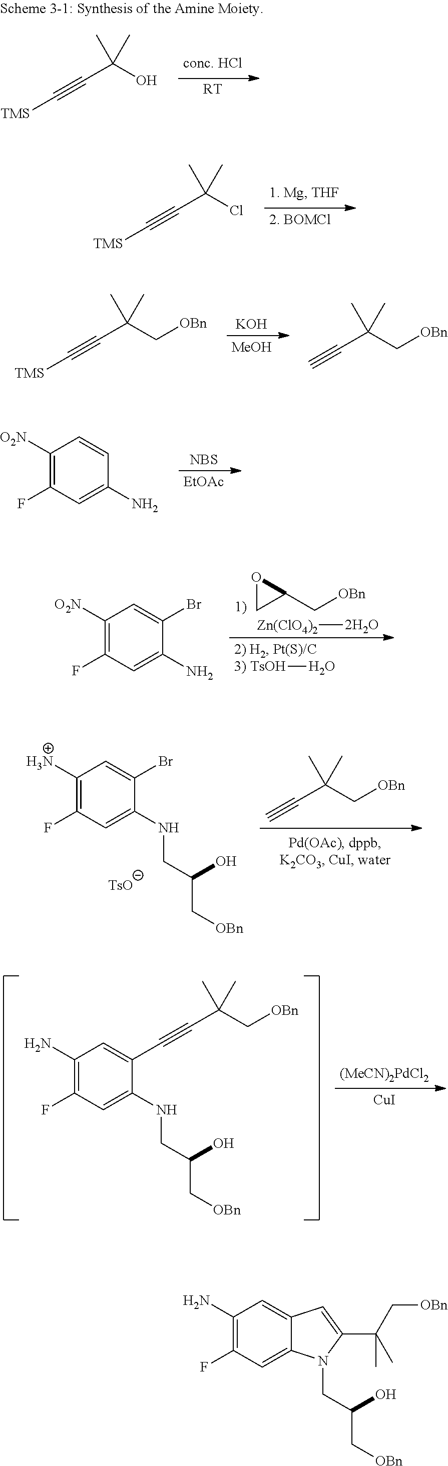

Scheme 3-1 provides an overview of the synthesis of the amine moiety of Compound 3. From the silyl protected propargyl alcohol shown, conversion to the propargyl chloride followed by formation of the Grignard reagent and subsequent nucleophilic substitution provides ((2,2-dimethylbut-3-ynyloxy)methyl)benzene, which is used in another step of the synthesis. To complete the amine moiety, 4-nitro-3-fluoroaniline is first brominated, and then converted to the toluenesulfonic acid salt of (R)-1-(4-amino-2-bromo-5-fluorophenylamino)-3-(benzyloxy)propan-2-ol in a two step process beginning with alkylation of the aniline amino group by (R)-2-(benzyloxymethyl)oxirane, followed by reduction of the nitro group to the corresponding amine. Palladium catalyzed coupling of the product with ((2,2-dimethylbut-3-ynyloxy)methyl)benzene (discussed above) provides the intermediate akynyl compound which is then cyclized to the indole moiety to produce the benzyl protected amine moiety of Compound 3.

II.C.3.c. Synthesis of Compound 3 by Acid and Amine Moiety Coupling

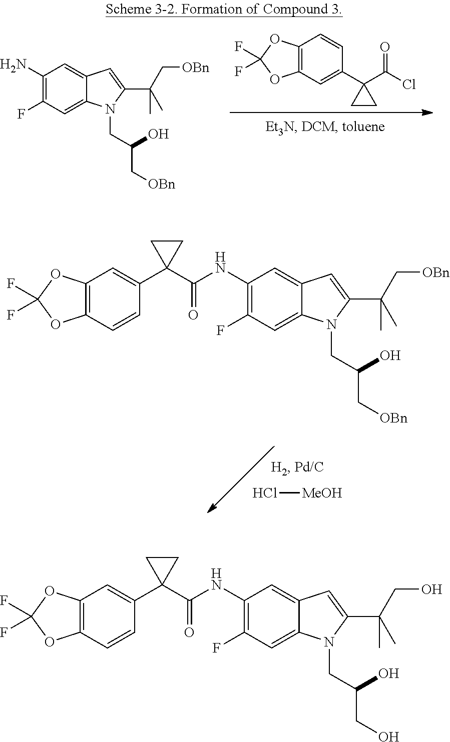

Scheme 3-2 depicts the coupling of the Acid and Amine moieties to produce Compound 3. In the first step, (R)-1-(5-amino-2-(1-(benzyloxy)-2-methylpropan-2-yl)-6-fluoro-1H-indol-1-yl)-3-(benzyloxy)propan-2-ol is coupled with 1-(2,2-difluorobenzo[d][1,3]dioxol-5-yl)cyclopropanecarbonyl chloride to provide the benzyl protected Compound 3. This step can be performed in the presence of a base and a solvent. The base can be an organic base such as triethylamine, and the solvent can be an organic solvent such as DCM or a mixture of DCM and toluene.

In the last step, the benzylated intermediate is deprotected to produce Compound 3. The deprotection step can be accomplished using reducing conditions sufficient to remove the benzyl group. The reducing conditions can be hydrogenation conditions such as hydrogen gas in the presence of a palladium catalyst.

### II.C.4. Examples: Synthesis of Compound 3

II.C.4.a. Compound 3 Amine Moiety Synthesis

### Example 3a

### 2-Bromo-5-fluoro-4-nitroaniline

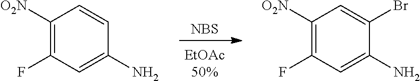

A flask was charged with 3-fluoro-4-nitroaniline (1.0 equiv) followed by ethyl acetate (10 vol) and stirred to dissolve all solids. N-Bromosuccinimide (1.0 equiv) was added portion-wise as to maintain an internal temperature of 22° C. At the end of the reaction, the reaction mixture was concentrated in vacuo on a rotavap. The residue was slurried in distilled water (5 vol) to dissolve and remove succinimide. (The succinimide can also be removed by water workup procedure.) The water was decanted and the solid was slurried in 2-propanol (5 vol) overnight. The resulting slurry was filtered and the wetcake was washed with 2-propanol, dried in vacuum oven at 50° C. overnight with N₂ bleed until constant weight was achieved. A yellowish tan solid was isolated (50% yield, 97.5% AUC). Other impurities were a bromo-regioisomer (1.4% AUC) and a di-bromo adduct (1.1% AUC). ¹H NMR (500 MHz, DMSO) δ 8.19 (1H, d, J=8.1 Hz), 7.06 (br. s, 2H), 6.64 (d, 1H, J=14.3 Hz).

### Example 3b

### p-toluenesulfonic acid salt of (R)-1-((4-amino-2-bromo-5-fluorophenyl)amino)-3-(benzyloxy)propan-2-ol

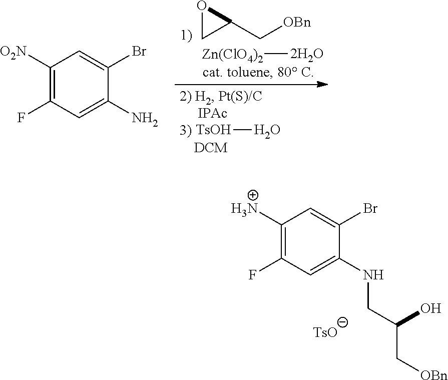

A thoroughly dried flask under N₂ was charged with the following: Activated powdered 4 Å molecular sieves (50 wt % based on 2-bromo-5-fluoro-4-nitroaniline), 2-Bromo-5-fluoro-4-nitroaniline (1.0 equiv), zinc perchlorate dihydrate (20 mol %), and toluene (8 vol). The mixture was stirred at room temperature for no more than 30 min. Lastly, (R)-benzyl glycidyl ether (2.0 equiv) in toluene (2 vol) was added in a steady stream. The reaction was heated to 80° C. (internal temperature) and stirred for approximately 7 hours or until 2-bromo-5-fluoro-4-nitroaniline was <5% AUC.

The reaction was cooled to room temperature and Celite® (50 wt %) was added, followed by ethyl acetate (10 vol). The resulting mixture was filtered to remove Celite® and sieves and washed with ethyl acetate (2 vol). The filtrate was washed with ammonium chloride solution (4 vol, 20% w/v). The organic layer was washed with sodium bicarbonate solution (4 vol×2.5% w/v). The organic layer was concentrated in vacuo on a rotovap. The resulting slurry was dissolved in isopropyl acetate (10 vol) and this solution was transferred to a Buchi hydrogenator.

The hydrogenator was charged with 5 wt % Pt(S)/C (1.5 mol %) and the mixture was stirred under N₂ at 30° C. (internal temperature). The reaction was flushed with N₂ followed by hydrogen. The hydrogenator pressure was adjusted to 1 Bar of hydrogen and the mixture was stirred rapidly (>1200 rpm). At the end of the reaction, the catalyst was filtered through a pad of Celite® and washed with dichloromethane (10 vol). The filtrate was concentrated in vacuo. Any remaining isopropyl acetate was chased with dichloromethane (2 vol) and concentrated on a rotavap to dryness.

The resulting residue was dissolved in dichloromethane (10 vol). p-Toluenesulfonic acid monohydrate (1.2 equiv) was added and stirred overnight. The product was filtered and washed with dichloromethane (2 vol) and suction dried. The wetcake was transferred to drying trays and into a vacuum oven and dried at 45° C. with N₂ bleed until constant weight was achieved. The p-toluenesulfonic acid salt of (R)-1-((4-amino-2-bromo-5-fluorophenyl)amino)-3-(benzyloxy)propan-2-ol was isolated as an off-white solid.

### Example 3c

### (3-Chloro-3-methylbut-1-ynyl)trimethylsilane

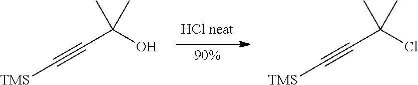

Propargyl alcohol (1.0 equiv) was charged to a vessel. Aqueous hydrochloric acid (37%, 3.75 vol) was added and stirring begun. During dissolution of the solid alcohol, a modest endotherm (5-6° C.) was observed. The resulting mixture was stirred overnight (16 h), slowly becoming dark red. A 30 L jacketed vessel was charged with water (5 vol) which was then cooled to 10° C. The reaction mixture was transferred slowly into the water by vacuum, maintaining the internal temperature of the mixture below 25° C. Hexanes (3 vol) was added and the resulting mixture was stirred for 0.5 h. The phases were settled and the aqueous phase (pH<1) was drained off and discarded. The organic phase was concentrated in vacuo using a rotary evaporator, furnishing the product as red oil.

### Example 3d

### (4-(Benzyloxy)-3,3-dimethylbut-1-ynyl)trimethylsilane

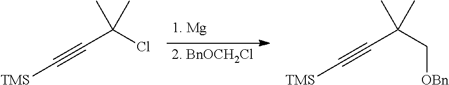

### Method A

All equivalents and volume descriptors in this part are based on a 250 g reaction. Magnesium turnings (69.5 g, 2.86 mol, 2.0 equiv) were charged to a 3 L 4-neck reactor and stirred with a magnetic stirrer under nitrogen for 0.5 h. The reactor was immersed in an ice-water bath. A solution of the propargyl chloride (250 g, 1.43 mol, 1.0 equiv) in THF (1.8 L, 7.2 vol) was added slowly to the reactor, with stirring, until an initial exotherm (about 10° C.) was observed. The Grignard reagent formation was confirmed by IPC using ¹H-NMR spectroscopy. Once the exotherm subsided, the remainder of the solution was added slowly, maintaining the batch temperature <15° C. The addition required about 3.5 h. The resulting dark green mixture was decanted into a 2 L capped bottle.

All equivalent and volume descriptors in this part are based on a 500 g reaction. A 22 L reactor was charged with a solution of benzyl chloromethyl ether (95%, 375 g, 2.31 mol, 0.8 equiv) in THF (1.5 L, 3 vol). The reactor was cooled in an ice-water bath. Two Grignard reagent batches prepared as above were combined and then added slowly to the benzyl chloromethyl ether solution via an addition funnel, maintaining the batch temperature below 25° C. The addition required 1.5 h. The reaction mixture was stirred overnight (16 h).

All equivalent and volume descriptors in this part are based on a 1 kg reaction. A solution of 15% ammonium chloride was prepared in a 30 L jacketed reactor (1.5 kg in 8.5 kg of water, 10 vol). The solution was cooled to 5° C. Two Grignard reaction mixtures prepared as above were combined and then transferred into the ammonium chloride solution via a header vessel. An exotherm was observed in this quench, which was carried out at a rate such as to keep the internal temperature below 25° C. Once the transfer was complete, the vessel jacket temperature was set to 25° C. Hexanes (8 L, 8 vol) was added and the mixture was stirred for 0.5 h. After settling the phases, the aqueous phase (pH 9) was drained off and discarded. The remaining organic phase was washed with water (2 L, 2 vol). The organic phase was concentrated in vacuo using a 22 L rotary evaporator, providing the crude product as an orange oil.

### Method B

Magnesium turnings (106 g, 4.35 mol, 1.0 eq) were charged to a 22 L reactor and then suspended in THF (760 mL, 1 vol). The vessel was cooled in an ice-water bath such that the batch temperature reached 2° C. A solution of the propargyl chloride (760 g, 4.35 mol, 1.0 equiv) in THF (4.5 L, 6 vol) was added slowly to the reactor. After 100 mL was added, the addition was stopped and the mixture stirred until a 13° C. exotherm was observed, indicating the Grignard reagent initiation. Once the exotherm subsided, another 500 mL of the propargyl chloride solution was added slowly, maintaining the batch temperature <20° C. The Grignard reagent formation was confirmed by IPC using ¹H-NMR spectroscopy. The remainder of the propargyl chloride solution was added slowly, maintaining the batch temperature <20° C. The addition required about 1.5 h. The resulting dark green solution was stirred for 0.5 h. The Grignard reagent formation was confirmed by IPC using ¹H-NMR spectroscopy. Neat benzyl chloromethyl ether was charged to the reactor addition funnel and then added dropwise into the reactor, maintaining the batch temperature below 25° C. The addition required 1.0 h. The reaction mixture was stirred overnight. The aqueous work-up and concentration was carried out using the same procedure and relative amounts of materials as in Method A to give the product as an orange oil.

### Example 3e

### 4-Benzyloxy-3,3-dimethylbut-1-yne

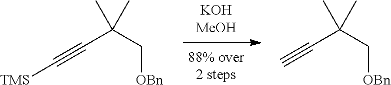

A 30 L jacketed reactor was charged with methanol (6 vol) which was then cooled to 5° C. Potassium hydroxide (85%, 1.3 equiv) was added to the reactor. A 15-20° C. exotherm was observed as the potassium hydroxide dissolved. The jacket temperature was set to 25° C. A solution of 4-benzyloxy-3,3-dimethyl-1-trimethylsilylbut-1-yne (1.0 equiv) in methanol (2 vol) was added and the resulting mixture was stirred until reaction completion, as monitored by HPLC. Typical reaction time at 25° C. was 3-4 h. The reaction mixture was diluted with water (8 vol) and then stirred for 0.5 h. Hexanes (6 vol) was added and the resulting mixture was stirred for 0.5 h. The phases were allowed to settle and then the aqueous phase (pH 10-11) was drained off and discarded. The organic phase was washed with a solution of KOH (85%, 0.4 equiv) in water (8 vol) followed by water (8 vol). The organic phase was then concentrated down using a rotary evaporator, yielding the title material as a yellow-orange oil. Typical purity of this material was in the 80% range with primarily a single impurity present. ¹H NMR (400 MHz, C₆D₆) δ 7.28 (d, 2H, J=7.4 Hz), 7.18 (t, 2H, J=7.2 Hz), 7.10 (d, 1H, J=7.2 Hz), 4.35 (s, 2H), 3.24 (s, 2H), 1.91 (s, 1H), 1.25 (s, 6H).

### Example 3f

### (R)-1-(4-amino-2-(4-(benzyloxy)-3,3-dimethylbut-1-ynyl)-5-fluorophenylamino)-3-(benzyloxy)propan-2-ol

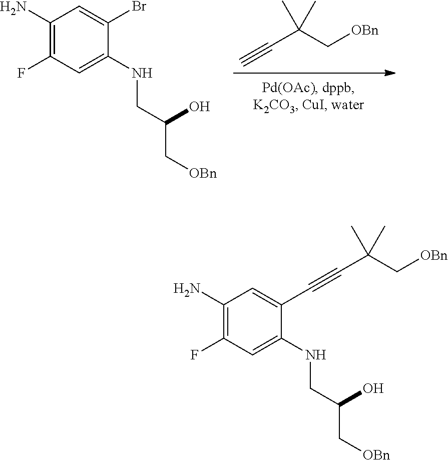

The tosylate salt of (R)-1-(4-amino-2-bromo-5-fluorophenylamino)-3-(benzyloxy)propan-2-ol was converted to the free base by stirring in dichloromethane (5 vol) and saturated NaHCO₃ solution (5 vol) until a clear organic layer was achieved. The resulting layers were separated and the organic layer was washed with saturated NaHCO₃ solution (5 vol) followed by brine and concentrated in vacuo to obtain (R)-1-(4-amino-2-bromo-5-fluorophenylamino)-3-(benzyloxy)propan-2-ol (free base) as an oil.

Palladium acetate (0.01 eq), dppb (0.015 eq), CuI (0.015 eq) and potassium carbonate (3 eq) were suspended in acetonitrile (1.2 vol). After stirring for 15 minutes, a solution of 4-benzyloxy-3,3-dimethylbut-1-yne (1.1 eq) in acetonitrile (0.2 vol) was added. The mixture was sparged with nitrogen gas for 1 h and then a solution of (R)-1-((4-amino-2-bromo-5-fluorophenyl)amino)-3-(benzyloxy)propan-2-ol free base (1 eq) in acetonitrile (4.1 vol) was added. The mixture was sparged with nitrogen gas for another hour and then was heated to 80° C. Reaction progress was monitored by HPLC and the reaction was usually complete within 3-5 h. The mixture was cooled to room temperature and then filtered through Celite. The cake was washed with acetonitrile (4 vol). The combined filtrates were azeotroped to dryness and then the mixture was polish filtered into the next reactor. The acetonitrile solution of (R)-1-β4-amino-2-(4-(benzyloxy)-3,3-dimethylbut-1-yn-1-yl)-5-fluorophenyl)amino)-3-(benzyloxy)propan-2-ol thus obtained was used directly in the next procedure (cyclization) without further purification.

### Example 3g

### (R)-1-(5-amino-2-(1-(benzyloxy)-2-methylpropan-2-yl)-6-fluoro-1H-indol-1-yl)-3-(benzyloxy)propan-2-ol

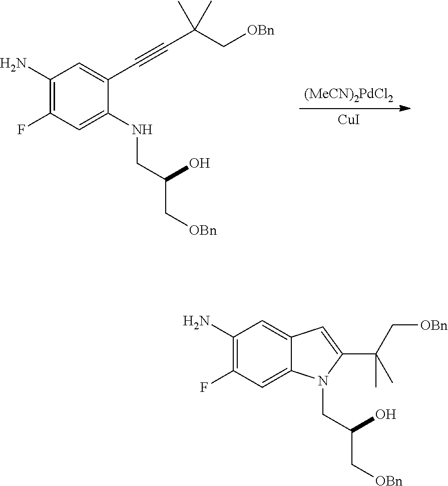

Bis-acetonitriledichloropalladium (0.1 eq) and CuI (0.1 eq) were charged to the reactor and then suspended in a solution of (R)-1-((4-amino-2-(4-(benzyloxy)-3,3-dimethylbut-1-yn-1-yl)-5-fluorophenyl)amino)-3-(benzyloxy)propan-2-ol obtained above (1 eq) in acetonitrile (9.5 vol total). The mixture was sparged with nitrogen gas for 1 h and then was heated to 80° C. The reaction progress was monitored by HPLC and the reaction was typically complete within 1-3h. The mixture was filtered through Celite and the cake was washed with acetonitrile. A solvent swap into ethyl acetate (7.5 vol) was performed. The ethyl acetate solution was washed with aqueous NH₃—NH₄Cl solution (2×2.5 vol) followed by 10% brine (2.5 vol). The ethyl acetate solution was then stirred with silica gel (1.8 wt eq) and Si-TMT (0.1 wt eq) for 6 h. After filtration, the resulting solution was concentrated down. The residual oil was dissolved in DCM/heptane (4 vol) and then purified by column chromatography. The oil thus obtained was then crystallized from 25% EtOAc/heptane (4 vol). Crystalline (R)-1-(5-amino-2-(1-(benzyloxy)-2-methylpropan-2-yl)-6-fluoro-1H-indol-1-yl)-3-(benzyloxy)propan-2-ol was typically obtained in 27-38% yield. ¹H NMR (400 MHz, DMSO) 7.38-7.34 (m, 4H), 7.32-7.23 (m, 6H), 7.21 (d, 1 H, J=12.8 Hz), 6.77 (d, 1H, J=9.0 Hz), 6.06 (s, 1H), 5.13 (d, 1H, J=4.9 Hz), 4.54 (s, 2H), 4.46 (br. s, 2H), 4.45 (s, 2H), 4.33 (d, 1H, J=12.4 Hz), 4.09-4.04 (m, 2H), 3.63 (d, 1H, J=9.2 Hz), 3.56 (d, 1H, J=9.2 Hz), 3.49 (dd, 1H, J=9.8, 4.4 Hz), 3.43 (dd, 1H, J=9.8, 5.7 Hz), 1.40 (s, 6H).

II.C.4.b. Coupling

### Example 3h

### Synthesis of (R)—N-(1-(3-(benzyloxy)-2-hydroxypropyl)-2-(1-(benzyloxy)-2-methylpropan-2-yl)-6-fluoro-1H-indol-5-yl)-1-(2,2-difluorobenzo[d][1,3]dioxol-5-yl)cyclopropanecarboxamide

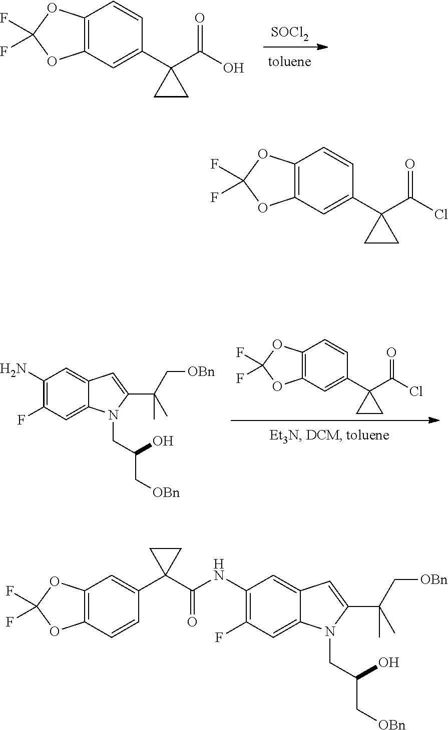

1-(2,2-Difluoro-1,3-benzodioxol-5-yl)-cyclopropanecarboxylic acid (1.3 equiv) was slurried in toluene (2.5 vol, based on 1-(2,2-difluoro-1,3-benzodioxol-5-yl)-cyclopropanecarboxylic acid). Thionyl chloride (SOCl₂, 1.7 equiv) was added via addition funnel and the mixture was heated to 60° C. The resulting mixture was stirred for 2 h. The toluene and the excess SOCl₂ were distilled off using a rotavop. Additional toluene (2.5 vol, based on 1-(2,2-difluoro-1,3-benzodioxol-5-yl)-cyclopropanecarboxylic acid) was added and the mixture was distilled down to 1 vol of toluene. A solution of (R)-1-(5-amino-2-(1-(benzyloxy)-2-methylpropan-2-yl)-6-fluoro-1H-indol-1-yl)-3-(benzyloxy)propan-2-ol (1 eq) and triethylamine (3 eq) in DCM (4 vol) was cooled to 0° C. The acid chloride solution in toluene (1 vol) was added while maintaining the batch temperature below 10° C. The reaction progress was monitored by HPLC, and the reaction was usually complete within minutes. After warming to 25° C., the reaction mixture was washed with 5% NaHCO₃ (3.5 vol), 1 M NaOH (3.5 vol) and 1 M HCl (5 vol). A solvent swap to into methanol (2 vol) was performed and the resulting solution of (R)—N-(1-(3-(benzyloxy)-2-hydroxypropyl)-2-(1-(benzyloxy)-2-methylpropan-2-yl)-6-fluoro-1H-indol-5-yl)-1-(2,2-difluorobenzo[d][1,3]dioxol-5-yl)cyclopropanecarboxamide in methanol was used without further purification in the next step (hydrogenolysis).

### Example 3i

### Synthesis of Compound 3

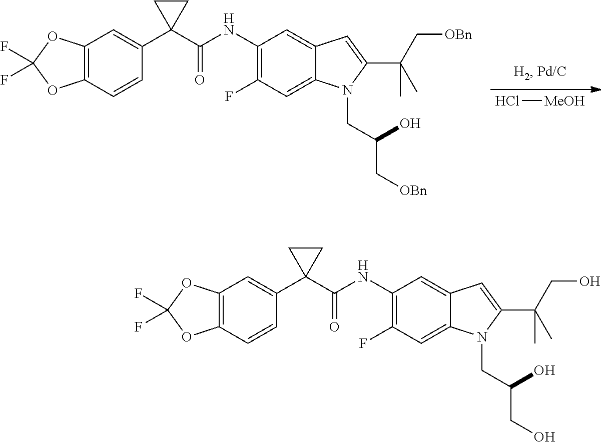

5% palladium on charcoal (˜50% wet, 0.01 eq) was charged to an appropriate hydrogenation vessel. The (R)—N-(1-(3-(benzyloxy)-2-hydroxypropyl)-2-(1-(benzyloxy)-2-methylpropan-2-yl)-6-fluoro-1H-indol-5-yl)-1-(2,2-difluorobenzo[d][1,3]dioxol-5-yl)cyclopropanecarboxamide solution in methanol (2 vol) obtained above was added carefully, followed by a 3 M solution of HCl in methanol. The vessel was purged with nitrogen gas and then with hydrogen gas. The mixture was stirred vigorously until the reaction was complete, as determined by HPLC analysis. Typical reaction time was 3-5 h. The reaction mixture was filtered through Celite and the cake was washed with methanol (2 vol). A solvent swap into isopropanol (3 vol) was performed. Crude Compound 3 was crystallized from 75% IPA-heptane (4 vol, ie. 1 vol heptane added to the 3 vol of IPA) and the resulting crystals were matured in 50% IPA-heptane (ie. 2 vol of heptane added to the mixture). Typical yields of Compound 3 from the two-step acylation/hydrogenolysis procedure range from 68% to 84%. Compound 3 can be recrystallized from IPA-heptane following the same procedure just described.

Compound 3 may also be prepared by one of several synthetic routes disclosed in US published patent application US 2009/0131492, incorporated herein by reference.

TABLE 3-1
Physical Data for Compound 3.

| Cmpd. | LC/MS | LC/RT | |
|---|---|---|---|
| No. | M + 1 | min | NMR |
| 3 | 521.5 | 1.69 | 1H NMR (400.0 MHz, CD₃CN) d 7.69 (d, J = 7.7 Hz, |
| | | | 1H), 7.44 (d, J = 1.6 Hz, 1H), 7.39 (dd, J = 1.7, 8.3 Hz, |
| | | | 1H), 7.31 (s, 1H), 7,27 (d, J = 8.3 Hz, 1H), 7.20 (d, J = |
| | | | 12.0 Hz, 1H), 6,34 (s, 1H), 4.32 (d, J = 6.8 Hz, 2H), |
| | | | 4.15-4.09 (m, 1H), 3.89 (dd, J = 6.0, 11.5 Hz, 1H), 3.63-3.52 |
| | | | (m, 3H), 3.42 (d, J = 4.6 Hz, 1H), 3.21 (dd, J = 6.2, 7.2 |
| | | | Hz, 1H), 3.04 (t, J = 5.8 Hz, 1H), 1.59 (dd, J = 3.8, 6.8 Hz, |
| | | | 2H), 1.44 (s, 3H), 1.33 (s, 3H) and 1.18 (dd, J = 3.7, 6.8 |
| | | | Hz, 2H) ppm. |

### III. Uses, Formulation and Administration

In one aspect, the invention features a formulation comprising a compound of Formula I, or a pharmaceutically acceptable salt thereof. In one embodiment of this aspect, the formulation includes a composition comprising a compound of Formula I, or a pharmaceutically acceptable salt thereof, and a pharmaceutically acceptable carrier or adjuvant.

In another aspect, the invention features a formulation comprising a component selected from any embodiment described in Column A of Table I in combination with a component selected from any embodiment described in Column B and/or a component selected from any embodiment described in Column C of Table I.

Table I is reproduced here for convenience.

TABLE I

| Column A | | Column B | | Column C | |
|---|---|---|---|---|---|
| Embodiments | | Embodiments | | Embodiments | |
| Section | Heading | Section | Heading | Section | Heading |
| II.A.1. | Compounds of | II.B.1. | Compounds of | II.C.1. | Compounds of |
| | Formula I | | Formula II | | Formula III |
| II.A.2. | Compound 1 | II.B.2. | Compound 2 | II.C.2. | Compound 3 |

In one embodiment of this aspect, the formulation comprises an embodiment described in Column A in combination with an embodiment described in Column B. In another embodiment, the formulation comprises an embodiment described in Column A in combination with an embodiment described in Column C. In another embodiment, the formulation comprises a combination of an embodiment described in Column A, an embodiment described in Column B, and an embodiment described in Column C.

In another embodiment of this aspect, the Column A component is a compound of Formula I. In another embodiment, the Column A component is Compound 1.

In another embodiment of this aspect, the Column B component is a compound of Formula II. In another embodiment, the Column B component is Compound 2.

In another embodiment of this aspect, the Column C component is a compound of Formula III. In another embodiment, the Column C component is Compound 3.

In one embodiment, the formulation comprises a homogeneous mixture comprising a composition according to Table I. In another embodiment, the formulation comprises a non-homogeneous mixture comprising a composition according to Table I. In some embodiments, the pharmaceutical composition of Table I can be administered in one vehicle or separately.

### III.A. Pharmaceutically Acceptable Compositions

In one aspect of the present invention, pharmaceutically acceptable compositions are provided, wherein these compositions comprise any of the compounds as described herein, and optionally comprise a pharmaceutically acceptable carrier, adjuvant or vehicle. In certain embodiments, these compositions optionally further comprise one or more additional therapeutic agents.

It will also be appreciated that certain of the compounds of present invention can exist in free form for treatment, or where appropriate, as a pharmaceutically acceptable derivative or a prodrug thereof. According to the present invention, a pharmaceutically acceptable derivative or a prodrug includes, but is not limited to, pharmaceutically acceptable salts, esters, salts of such esters, or any other adduct or derivative which upon administration to a patient in need thereof is capable of providing, directly or indirectly, a compound as otherwise described herein, or a metabolite or residue thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. A “pharmaceutically acceptable salt” means any non-toxic salt or salt of an ester of a compound of this invention that, upon administration to a recipient, is capable of providing, either directly or indirectly, a compound of this invention or an inhibitorily active metabolite or residue thereof.

Pharmaceutically acceptable salts are well known in the art. For example, S. M. Berge, et al. describes pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange.

Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, edisylate (ethanedisulfonate), ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. This invention also envisions the quaternization of any basic nitrogen-containing groups of the compounds disclosed herein. Water or oil-soluble or dispersible products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

As described above, the pharmaceutically acceptable compositions of the present invention additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

### III.B. Uses of Compounds and Pharmaceutically Acceptable Compositions

In yet another aspect, the present invention provides a method of treating or lessening the severity of a condition, disease, or disorder implicated by CFTR mutation. In certain embodiments, the present invention provides a method of treating a condition, disease, or disorder implicated by a deficiency of the CFTR activity, the method comprising administering a composition comprising a compound of Formula I to a subject, preferably a mammal, in need thereof.

In certain embodiments, the present invention provides a method of treating a condition, disease, or disorder implicated by a deficiency of the CFTR activity, the method comprising administering a composition comprising an embodiment described in Column A in combination with an embodiment described in Column B of Table I. In another embodiment, the formulation comprises an embodiment described in Column A in combination with an embodiment described in Column C of Table I. In another embodiment, the formulation comprises a combination of an embodiment described in Column A, an embodiment described in Column B, and an embodiment described in Column C of Table I.

In another embodiment of this aspect, the Column A component is a compound of Formula I. In another embodiment, the Column A component is Compound 1.

In another embodiment of this aspect, the Column B component is a compound of Formula II. In another embodiment, the Column B component is Compound 2.

In another embodiment of this aspect, the Column C component is a compound of Formula III. In another embodiment, the Column C component is Compound 3.

In certain embodiments, the present invention provides a method of treating diseases associated with reduced CFTR function due to mutations in the gene encoding CFTR or environmental factors (e.g., smoke). These diseases include, cystic fibrosis, chronic bronchitis, recurrent bronchitis, acute bronchitis, male infertility caused by congenital bilateral absence of the vas deferens (CBAVD), female infertility caused by congenital absence of the uterus and vagina (CAUV), idiopathic chronic pancreatitis (ICP), idiopathic recurrent pancreatitis, idiopathic acute pancreatitis, chronic rhinosinusitis, primary sclerosing cholangitis, allergic bronchopulmonary aspergillosis, diabetes, dry eye, constipation, allergic bronchopulmonary aspergillosis (ABPA), bone diseases (e.g., osteoporosis), and asthma.

In certain embodiments, the present invention provides a method for treating diseases associated with normal CFTR function. These diseases include, chronic obstructive pulmonary disease (COPD), chronic bronchitis, recurrent bronchitis, acute bronchitis, rhinosinusitis, constipation, pancreatitis including chronic pancreatitis, recurrent pancreatitis, and acute pancreatitis, pancreatic insufficiency, male infertility caused by congenital bilateral absence of the vas deferens (CBAVD), mild pulmonary disease, idiopathic pancreatitis, liver disease, hereditary emphysema, gallstones, gasgtroesophageal reflux disease, gastrointestinal malignancies, inflammatory bowel disease, constipation, diabetes, arthritis, osteoporosis, and osteopenia.

In certain embodiments, the present invention provides a method for treating diseases associated with normal CFTR function including hereditary hemochromatosis, coagulation-fibrinolysis deficiencies, such as protein C deficiency, Type 1 hereditary angioedema, lipid processing deficiencies, such as familial hypercholesterolemia, Type 1 chylomicronemia, abetalipoproteinemia, lysosomal storage diseases, such as I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myleoperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases such as Alzheimer's disease, Parkinson's disease, amyotrophic lateral sclerosis, progressive supranuclear palsy, Pick's disease, several polyglutamine neurological disorders such as Huntington's, spinocerebullar ataxia type I, spinal and bulbar muscular atrophy, dentatorubal pallidoluysian, and myotonic dystrophy, as well as spongiform encephalopathies, such as hereditary Creutzfeldt-Jakob disease (due to prion protein processing defect), Fabry disease, Straussler-Scheinker syndrome, Gorham's Syndrome, chloride channelopathies, myotonia congenita (Thomson and Becker forms), Bartter's syndrome type III, Dent's disease, hyperekplexia, epilepsy, lysosomal storage disease, Angelman syndrome, Primary Ciliary Dyskinesia (PCD), PCD with situs inversus (also known as Kartagener syndrome), PCD without situs inversus and ciliary aplasia, or Sjogren's disease, comprising the step of administering to said mammal an effective amount of a composition comprising a compound of the present invention.

In certain embodiments, the present invention provides a method of treating a condition, disease, or disorder implicated by a deficiency of CFTR activity, the method comprising administering the pharmaceutical composition of the invention to a subject, preferably a mammal, in need thereof.

In yet another aspect, the present invention provides a method of treating, or lessening the severity of a condition, disease, or disorder implicated by CFTR mutation. In certain embodiments, the present invention provides a method of treating a condition, disease, or disorder implicated by a deficiency of the CFTR activity, the method comprising administering the pharmaceutical composition of the invention to a subject, preferably a mammal, in need thereof.

In another aspect, the invention also provides a method of treating or lessening the severity of a disease in a patient, the method comprising administering the pharmaceutical composition of the invention to a subject, preferably a mammal, in need thereof, and said disease is selected from cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, idiopathic pancreatitis, allergic bronchopulmonary aspergillosis (ABPA), liver disease, hereditary emphysema, hereditary hemochromatosis, coagulation-fibrinolysis deficiencies, such as protein C deficiency, Type 1 hereditary angioedema, lipid processing deficiencies, such as familial hypercholesterolemia, Type 1 chylomicronemia, abetalipoproteinemia, lysosomal storage diseases, such as I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myleoperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases such as Alzheimer's disease, Parkinson's disease, amyotrophic lateral sclerosis, progressive supranuclear palsy, Pick's disease, several polyglutamine neurological disorders such as Huntington's, spinocerebullar ataxia type I, spinal and bulbar muscular atrophy, dentatorubal pallidoluysian, and myotonic dystrophy, as well as spongiform encephalopathies, such as hereditary Creutzfeldt-Jakob disease (due to prion protein processing defect), Fabry disease, Straussler-Scheinker syndrome, COPD, dry-eye disease, or Sjogren's disease, Osteoporosis, Osteopenia, bone healing and bone growth (including bone repair, bone regeneration, reducing bone resorption and increasing bone deposition), Gorham's Syndrome, chloride channelopathies such as myotonia congenita (Thomson and Becker forms), Bartter's syndrome type III, Dent's disease, hyperekplexia, epilepsy, lysosomal storage disease, Angelman syndrome, and Primary Ciliary Dyskinesia (PCD), a term for inherited disorders of the structure and/or function of cilia, including PCD with situs inversus (also known as Kartagener syndrome), PCD without situs inversus and ciliary aplasia.

In some embodiments, the method includes treating or lessening the severity of cystic fibrosis in a patient comprising administering to said patient one of the compositions as defined herein. In certain embodiments, the patient possesses mutant forms of human CFTR. In other embodiments, the patient possesses one or more of the following mutations ΔF508, R117H, and G551D of human CFTR. In one embodiment, the method includes treating or lessening the severity of cystic fibrosis in a patient possessing the ΔF508 mutation of human CFTR comprising administering to said patient one of the compositions as defined herein. In one embodiment, the method includes treating or lessening the severity of cystic fibrosis in a patient possessing the G551D mutation of human CFTR comprising administering to said patient one of the compositions as defined herein. In one embodiment, the method includes treating or lessening the severity of cystic fibrosis in a patient possessing the ΔF508 mutation of human CFTR on at least one allele comprising administering to said patient one of the compositions as defined herein. In one embodiment, the method includes treating or lessening the severity of cystic fibrosis in a patient possessing the ΔF508 mutation of human CFTR on both alleles comprising administering to said patient one of the compositions as defined herein. In one embodiment, the method includes treating or lessening the severity of cystic fibrosis in a patient possessing the G551D mutation of human CFTR on at least one allele comprising administering to said patient one of the compositions as defined herein. In one embodiment, the method includes treating or lessening the severity of cystic fibrosis in a patient possessing the G551D mutation of human CFTR on both alleles comprising administering to said patient one of the compositions as defined herein.

In some embodiments, the method includes lessening the severity of cystic fibrosis in a patient comprising administering to said patient one of the compositions as defined herein. In certain embodiments, the patient possesses mutant forms of human CFTR. In other embodiments, the patient possesses one or more of the following mutations ΔF508, R117H, and G551D of human CFTR. In one embodiment, the method includes lessening the severity of cystic fibrosis in a patient possessing the ΔF508 mutation of human CFTR comprising administering to said patient one of the compositions as defined herein. In one embodiment, the method includes lessening the severity of cystic fibrosis in a patient possessing the G551D mutation of human CFTR comprising administering to said patient one of the compositions as defined herein. In one embodiment, the method includes lessening the severity of cystic fibrosis in a patient possessing the ΔF508 mutation of human CFTR on at least one allele comprising administering to said patient one of the compositions as defined herein. In one embodiment, the method includes lessening the severity of cystic fibrosis in a patient possessing the ΔF508 mutation of human CFTR on both alleles comprising administering to said patient one of the compositions as defined herein. In one embodiment, the method includes lessening the severity of cystic fibrosis in a patient possessing the G551D mutation of human CFTR on at least one allele comprising administering to said patient one of the compositions as defined herein. In one embodiment, the method includes lessening the severity of cystic fibrosis in a patient possessing the G551D mutation of human CFTR on both alleles comprising administering to said patient one of the compositions as defined herein.

In some aspects, the invention provides a method of treating or lessening the severity of Osteoporosis in a patient comprising administering to said patient a composition as defined herein.

In certain embodiments, the method of treating or lessening the severity of Osteoporosis in a patient comprises administering to said patient a pharmaceutical composition as described herein.

In some aspects, the invention provides a method of treating or lessening the severity of Osteopenia in a patient comprising administering to said patient a composition as defined herein.

In certain embodiments, the method of treating or lessening the severity of Osteopenia in a patient comprises administering to said patient a pharmaceutical composition as described herein.

In some aspects, the invention provides a method of bone healing and/or bone repair in a patient comprising administering to said patient a composition as defined herein.

In certain embodiments, the method of bone healing and/or bone repair in a patient comprises administering to said patient a pharmaceutical composition as described herein.

In some aspects, the invention provides a method of reducing bone resorption in a patient comprising administering to said patient a composition as defined herein.

In some aspects, the invention provides a method of increasing bone deposition in a patient comprising administering to said patient a composition as defined herein.

In certain embodiments, the method of increasing bone deposition in a patient comprises administering to said patient a composition as defined herein.

In some aspects, the invention provides a method of treating or lessening the severity of COPD in a patient comprising administering to said patient a composition as defined herein.

In certain embodiments, the method of treating or lessening the severity of COPD in a patient comprises administering to said patient a composition as defined herein.

In some aspects, the invention provides a method of treating or lessening the severity of smoke induced COPD in a patient comprising administering to said patient a composition as defined herein.

In certain embodiments, the method of treating or lessening the severity of smoke induced COPD in a patient comprises administering to said patient a composition as defined herein.

In some aspects, the invention provides a method of treating or lessening the severity of chronic bronchitis in a patient comprising administering to said patient a composition as described herein.

In certain embodiments, the method of treating or lessening the severity of chronic bronchitis in a patient comprises administering to said patient a composition as defined herein.

According to an alternative preferred embodiment, the present invention provides a method of treating cystic fibrosis comprising the step of administering to said mammal a composition comprising the step of administering to said mammal an effective amount of a composition comprising a compound of the present invention.

According to the invention an “effective amount” of the compound or pharmaceutically acceptable composition is that amount effective for treating or lessening the severity of one or more of the diseases, disorders or conditions as recited above.

The compounds and compositions, according to the method of the present invention, may be administered using any amount and any route of administration effective for treating or lessening the severity of one or more of the diseases, disorders or conditions as recited above.

In certain embodiments, the compounds and compositions of the present invention are useful for treating or lessening the severity of cystic fibrosis in patients who exhibit residual CFTR activity in the apical membrane of respiratory and non-respiratory epithelia. The presence of residual CFTR activity at the epithelial surface can be readily detected using methods known in the art, e.g., standard electrophysiological, biochemical, or histochemical techniques. Such methods identify CFTR activity using in vivo or ex vivo electrophysiological techniques, measurement of sweat or salivary Cl⁻ concentrations, or ex vivo biochemical or histochemical techniques to monitor cell surface density. Using such methods, residual CFTR activity can be readily detected in patients heterozygous or homozygous for a variety of different mutations, including patients homozygous or heterozygous for the most common mutation, ΔF508.

In another embodiment, the compounds and compositions of the present invention are useful for treating or lessening the severity of cystic fibrosis in patients who have residual CFTR activity induced or augmented using pharmacological methods or gene therapy. Such methods increase the amount of CFTR present at the cell surface, thereby inducing a hitherto absent CFTR activity in a patient or augmenting the existing level of residual CFTR activity in a patient.

In one embodiment, the compounds and compositions of the present invention are useful for treating or lessening the severity of cystic fibrosis in patients within certain genotypes exhibiting residual CFTR activity, e.g., class III mutations (impaired regulation or gating), class IV mutations (altered conductance), or class V mutations (reduced synthesis) (Lee R. Choo-Kang, Pamela L., Zeitlin, Type I, II, III, IV, and V cystic fibrosis Transmembrane Conductance Regulator Defects and Opportunities of Therapy; Current Opinion in Pulmonary Medicine 6:521-529, 2000). Other patient genotypes that exhibit residual CFTR activity include patients homozygous for one of these classes or heterozygous with any other class of mutations, including class I mutations, class II mutations, or a mutation that lacks classification.

In one aspect, the invention includes a method of treating a class III mutation as described above, comprising administering to a patient in need thereof a composition comprising a compound of Formula I in combination with one or both of a compound of Formula II and/or a compound of Formula III. In some embodiments of this aspect, the composition includes a compound of Formula I in combination with a compound of Formula II. In some embodiments of this aspect, the composition includes a compound of Formula I in combination with a compound of Formula III. In some embodiments of this aspect, the composition includes a compound of Formula I in combination with a compound of Formula II and a compound of Formula III. In a further embodiment of this aspect, the pharmaceutical composition includes Compound 1 and Compound 2. In another embodiment, the pharmaceutical composition includes Compound 1 and Compound 3. In another embodiment, the pharmaceutical composition includes Compound 1, Compound 2 and Compound 3.

In one embodiment, the compounds and compositions of the present invention are useful for treating or lessening the severity of cystic fibrosis in patients within certain clinical phenotypes, e.g., a moderate to mild clinical phenotype that typically correlates with the amount of residual CFTR activity in the apical membrane of epithelia. Such phenotypes include patients exhibiting pancreatic insufficiency or patients diagnosed with idiopathic pancreatitis and congenital bilateral absence of the vas deferens, or mild lung disease.

The exact amount required will vary from subject to subject, depending on the species, age, and general condition of the subject, the severity of the infection, the particular agent, its mode of administration, and the like. The compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient,” as used herein, means an animal, preferably a mammal, and most preferably a human.

The pharmaceutically acceptable compositions of this invention can be administered to humans and other animals orally, rectally, parenterally, intracisternally, intravaginally, intraperitoneally, topically (as by powders, ointments, drops or patch), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 0.5 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms, the active compound may be admixed with at least one inert diluent such as sucrose, lactose or starch. Such dosage forms may also comprise, as is normal practice, additional substances other than inert diluents, e.g., tabletting lubricants and other tabletting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms are prepared by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The activity of a compound utilized in this invention as a modulator of CFTR may be assayed according to methods described generally in the art and in the Examples herein.

It will also be appreciated that the compounds and pharmaceutically acceptable compositions of the present invention can be employed in combination therapies, that is, the compounds and pharmaceutically acceptable compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another agent used to treat the same disorder), or they may achieve different effects (e.g., control of any adverse effects). As used herein, additional therapeutic agents that are normally administered to treat or prevent a particular disease, or condition, are known as “appropriate for the disease, or condition, being treated.”

In one embodiment, the additional agent is selected from a mucolytic agent, bronchodialator, an anti-biotic, an anti-infective agent, an anti-inflammatory agent, a CFTR modulator other than a Compound of the present invention, or a nutritional agent.

In one embodiment, the additional agent is an antibiotic. Exemplary antibiotics useful herein include tobramycin, including tobramycin inhaled powder (TIP), azithromycin, aztreonam, including the aerosolized form of aztreonam, amikacin, including liposomal formulations thereof, ciprofloxacin, including formulations thereof suitable for administration by inhalation, levoflaxacin, including aerosolized formulations thereof, and combinations of two antibiotics, e.g., fosfomycin and tobramycin.

In another embodiment, the additional agent is a mucolyte. Exemplary mucolytes useful herein includes Pulmozyme®.

In another embodiment, the additional agent is a bronchodialator. Exemplary bronchodialtors include albuterol, metaprotenerol sulfate, pirbuterol acetate, salmeterol, or tetrabuline sulfate.

In another embodiment, the additional agent is effective in restoring lung airway surface liquid. Such agents improve the movement of salt in and out of cells, allowing mucus in the lung airway to be more hydrated and, therefore, cleared more easily. Exemplary such agents include hypertonic saline, denufosol tetrasodium ([[(3S, 5R)-5-(4-amino-2-oxopyrimidin-1-yl)-3-hydroxyoxolan-2-yl]methoxy-hydroxyphosphoryl][[[(2R,3S,4R,5R)-5-(2,4-dioxopyrimidin-1-yl)-3,4-dihydroxyoxolan-2-yl]methoxy-hydroxyphosphoryl]oxy-hydroxyphosphoryl]hydrogen phosphate), or bronchitol (inhaled formulation of mannitol).

In another embodiment, the additional agent is an anti-inflammatory agent, i.e., an agent that can reduce the inflammation in the lungs. Exemplary such agents useful herein include ibuprofen, docosahexanoic acid (DHA), sildenafil, inhaled glutathione, pioglitazone, hydroxychloroquine, or simavastatin.

In another embodiment, the additional agent is a CFTR modulator other than Compound 1, i.e., an agent that has the effect of modulating CFTR activity. Exemplary such agents include ataluren (“PTC124®”; 3-[5-(2-fluorophenyl)-1,2,4-oxadiazol-3-yl]benzoic acid), sinapultide, lancovutide, depelestat (a human recombinant neutrophil elastase inhibitor), cobiprostone (7-{(2R,4aR,5R,7aR)-2-[(3S)-1,1-difluoro-3-methylpentyl]-2-hydroxy-6-oxooctahydrocyclopenta[b]pyran-5-yl}heptanoic acid), or (3-(6-(1-(2,2-difluorobenzo[d][1,3]dioxol-5-yl)cyclopropanecarboxamido)-3-methylpyridin-2-yl)benzoic acid. In another embodiment, the additional agent is (3-(6-(1-(2,2-difluorobenzo[d][1,3]dioxol-5-yl) cyclopropanecarboxamido)-3-methylpyridin-2-yl)benzoic acid.

In another embodiment, the additional agent is a nutritional agent. Exemplary such agents include pancrelipase (pancreating enzyme replacement), including Pancrease®, Pancreacarb®, Ultrase®, or Creon®, Liprotomase® (formerly Trizytek®), Aquadeks®, or glutathione inhalation. In one embodiment, the additional nutritional agent is pancrelipase.

Amongst other diseases described herein, combinations of CFTR modulators, such as compounds of Formula I, and agents that reduce the activity of ENaC are use for treating Liddle's syndrome, an inflammatory or allergic condition including cystic fibrosis, primary ciliary dyskinesia, chronic bronchitis, chronic obstructive pulmonary disease, asthma, respiratory tract infections, lung carcinoma, xerostomia and keratoconjunctivitis sire, respiratory tract infections (acute and chronic; viral and bacterial) and lung carcinoma.

Combinations of CFTR modulators, such as compounds of Formula I, and agents that reduce the activity of ENaC are also useful for treating diseases mediated by blockade of the epithelial sodium channel also include diseases other than respiratory diseases that are associated with abnormal fluid regulation across an epithelium, perhaps involving abnormal physiology of the protective surface liquids on their surface, e.g., xerostomia (dry mouth) or keratoconjunctivitis sire (dry eye). Furthermore, blockade of the epithelial sodium channel in the kidney could be used to promote diuresis and thereby induce a hypotensive effect.

Asthma includes both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g., of less than 4 or 5 years of age, exhibiting wheezing symptoms and diagnosed or diagnosable as “wheezy infants,” an established patient category of major medical concern and now often identified as incipient or early-phase asthmatics. (For convenience, this particular asthmatic condition is referred to as “wheezy-infant syndrome.”) Prophylactic efficacy in the treatment of asthma will be evidenced by reduced frequency or severity of symptomatic attack, e.g., of acute asthmatic or bronchoconstrictor attack, improvement in lung function or improved airways hyperreactivity. It may further be evidenced by reduced requirement for other, symptomatic therapy, i.e., therapy for or intended to restrict or abort symptomatic attack when it occurs, e.g., anti-inflammatory (e.g., cortico-steroid) or bronchodilatory. Prophylactic benefit in asthma may, in particular, be apparent in subjects prone to “morning dipping.” “Morning dipping” is a recognized asthmatic syndrome, common to a substantial percentage of asthmatics and characterized by asthma attack, e.g., between the hours of about 4-6 am, i.e., at a time normally substantially distant from any previously administered symptomatic asthma therapy.

Chronic obstructive pulmonary disease includes chronic bronchitis or dyspnea associated therewith, emphysema, as well as exacerbation of airways hyperreactivity consequent to other drug therapy, in particular, other inhaled drug therapy. In some embodiments, the combinations of CFTR modulators, such as compounds of Formula I, and agents that reduce the activity of ENaC are useful for the treatment of bronchitis of whatever type or genesis including, e.g., acute, arachidic, catarrhal, croupus, chronic or phthinoid bronchitis.

In another embodiment, the additional agent is a CFTR modulator other than a compound of formula I, i.e., an agent that has the effect of modulating CFTR activity. Exemplary such agents include ataluren (“PTC124®”; 3-[5-(2-fluorophenyl)-1,2,4-oxadiazol-3-yl]benzoic acid), sinapultide, lancovutide, depelestat (a human recombinant neutrophil elastase inhibitor), cobiprostone (7-{(2R,4aR,5R,7aR)-2-[(3S)-1,1-difluoro-3-methylpentyl]-2-hydroxy-6-oxooctahydrocyclopenta[b]pyran-5-yl}heptanoic acid), or (3-(6-(2,2-difluorobenzo[d][1,3]dioxol-5-yl)cyclopropanecarboxamido)-3-methylpyridin-2-yl)benzoic acid. In another embodiment, the additional agent is (3-(6-(1-(2,2-difluorobenzo[d][1,3]dioxol-5-yl) cyclopropanecarboxamido)-3-methylpyridin-2-yl) benzoic acid.

In another embodiment, the additional agent is a nutritional agent. Exemplary such agents include pancrelipase (pancreating enzyme replacement), including Pancrease®, Pancreacarb®, Ultrase®, or Creon®, Liprotomase® (formerly Trizytek®), Aquadeks®, or glutathione inhalation. In one embodiment, the additional nutritional agent is pancrelipase.

The amount of additional therapeutic agent present in the compositions of this invention will be no more than the amount that would normally be administered in a composition comprising that therapeutic agent as the only active agent. Preferably, the amount of additional therapeutic agent in the presently disclosed compositions will range from about 50% to 100% of the amount normally present in a composition comprising that agent as the only therapeutically active agent.

The compounds of this invention or pharmaceutically acceptable compositions thereof may also be incorporated into compositions for coating an implantable medical device, such as prostheses, artificial valves, vascular grafts, stents and catheters. Accordingly, the present invention, in another aspect, includes a composition for coating an implantable device comprising a compound of the present invention as described generally above, and in classes and subclasses herein, and a carrier suitable for coating said implantable device. In still another aspect, the present invention includes an implantable device coated with a composition comprising a compound of the present invention as described generally above, and in classes and subclasses herein, and a carrier suitable for coating said implantable device. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccarides, polyethylene glycol, phospholipids or combinations thereof to impart controlled release characteristics in the composition.

Another aspect of the invention relates to modulating CFTR activity in a biological sample or a patient (e.g., in vitro or in vivo), which method comprises administering to the patient, or contacting said biological sample with a compound of Formula I or a composition comprising said compound. The term “biological sample,” as used herein, includes, without limitation, cell cultures or extracts thereof; biopsied material obtained from a mammal or extracts thereof; and blood, saliva, urine, feces, semen, tears, or other body fluids or extracts thereof.

Modulation of CFTR in a biological sample is useful for a variety of purposes that are known to one of skill in the art. Examples of such purposes include, but are not limited to, the study of CFTR in biological and pathological phenomena; and the comparative evaluation of new modulators of CFTR.

In yet another embodiment, a method of modulating activity of an anion channel in vitro or in vivo, is provided comprising the step of contacting said channel with a compound of Formula (I). In preferred embodiments, the anion channel is a chloride channel or a bicarbonate channel. In other preferred embodiments, the anion channel is a chloride channel.

According to an alternative embodiment, the present invention provides a method of increasing the number of functional CFTR in a membrane of a cell, comprising the step of contacting said cell with a compound of Formula (I).

According to another preferred embodiment, the activity of the CFTR is measured by measuring the transmembrane voltage potential. Means for measuring the voltage potential across a membrane in the biological sample may employ any of the known methods in the art, such as optical membrane potential assay or other electrophysiological methods.

The optical membrane potential assay utilizes voltage-sensitive FRET sensors described by Gonzalez and Tsien (See, Gonzalez, J. E. and R. Y. Tsien (1995) “Voltage sensing by fluorescence resonance energy transfer in single cells.” Biophys J 69(4): 1272-80, and Gonzalez, J. E. and R. Y. Tsien (1997); “Improved indicators of cell membrane potential that use fluorescence resonance energy transfer” Chem Biol 4(4): 269-77) in combination with instrumentation for measuring fluorescence changes such as the Voltage/Ion Probe Reader (VIPR) (See, Gonzalez, J. E., K. Oades, et al. (1999) “Cell-based assays and instrumentation for screening ion-channel targets” Drug Discov Today 4(9): 431-439).

These voltage sensitive assays are based on the change in fluorescence resonant energy transfer (FRET) between the membrane-soluble, voltage-sensitive dye, DiSBAC₂(3), and a fluorescent phospholipid, CC2-DMPE, which is attached to the outer leaflet of the plasma membrane and acts as a FRET donor. Changes in membrane potential (Vₘ) cause the negatively charged DiSBAC₂(3) to redistribute across the plasma membrane and the amount of energy transfer from CC2-DMPE changes accordingly. The changes in fluorescence emission can be monitored using VIPR™ II, which is an integrated liquid handler and fluorescent detector designed to conduct cell-based screens in 96- or 384-well microtiter plates.

In another aspect the present invention provides a kit for use in measuring the activity of CFTR or a fragment thereof in a biological sample in vitro or in vivo comprising (i) a composition comprising a compound of Formula I or any of the above embodiments; and (ii) instructions for a) contacting the composition with the biological sample and b) measuring activity of said CFTR or a fragment thereof. In one embodiment, the kit further comprises instructions for a) contacting an additional composition with the biological sample; b) measuring the activity of said CFTR or a fragment thereof in the presence of said additional compound, and c) comparing the activity of the CFTR in the presence of the additional compound with the density of the CFTR in the presence of a composition of Formula (I). In preferred embodiments, the kit is used to measure the density of CFTR.

In order that the invention described herein may be more fully understood, the following examples are set forth. It should be understood that these examples are for illustrative purposes only and are not to be construed as limiting this invention in any manner.

### IV. Assays

### IV.A. Protocol 1: Assays for Detecting and Measuring ΔF508-CFTR Potentiation Properties of Compounds

### Membrane Potential Optical Methods for Assaying ΔF508-CFTR Modulation Properties of Compounds

The assay utilizes fluorescent voltage sensing dyes to measure changes in membrane potential using a fluorescent plate reader (e.g., FLIPR III, Molecular Devices, Inc.) as a readout for increase in functional ΔF508-CFTR in NIH 3T3 cells. The driving force for the response is the creation of a chloride ion gradient in conjunction with channel activation by a single liquid addition step after the cells have previously been treated with compounds and subsequently loaded with a voltage sensing dye.

### Identification of Potentiator Compounds

To identify potentiators of ΔF508-CFTR, a double-addition HTS assay format was developed. This HTS assay utilizes fluorescent voltage sensing dyes to measure changes in membrane potential on the FLIPR III as a measurement for increase in gating (conductance) of ΔF508 CFTR in temperature-corrected ΔF508 CFTR NIH 3T3 cells. The driving force for the response is a Cl⁻ ion gradient in conjunction with channel activation with forskolin in a single liquid addition step using a fluorescent plate reader such as FLIPR III after the cells have previously been treated with potentiator compounds (or DMSO vehicle control) and subsequently loaded with a redistribution dye.

### Solutions

Bath Solution #1: (in mM) NaCl 160, KCl 4.5, CaCl₂ 2, MgCl₂ 1, HEPES 10, pH 7.4 with NaOH.

Chloride-free bath solution: Chloride salts in Bath Solution #1 (above) are substituted with gluconate salts.

### Cell Culture

NIH3T3 mouse fibroblasts stably expressing ΔF508-CFTR are used for optical measurements of membrane potential. The cells are maintained at 37° C. in 5% CO₂ and 90% humidity in Dulbecco's modified Eagle's medium supplemented with 2 mM glutamine, 10% fetal bovine serum, 1×NEAA, β-ME, 1×pen/strep, and 25 mM HEPES in 175 cm² culture flasks. For all optical assays, the cells were seeded at ˜20,000/well in 384-well matrigel-coated plates and cultured for 2 hrs at 37° C. before culturing at 27° C. for 24 hrs. for the potentiator assay. For the correction assays, the cells are cultured at 27° C. or 37° C. with and without compounds for 16-24 hours. Electrophysiological Assays for assaying ΔF508-CFTR modulation properties of compounds.

### Ussing Chamber Assay

Ussing chamber experiments were performed on polarized airway epithelial cells expressing ΔF508-CFTR to further characterize the ΔF508-CFTR modulators identified in the optical assays. Non-CF and CF airway epithelia were isolated from bronchial tissue, cultured as previously described (Galietta, L. J. V., Lantero, S., Gazzolo, A., Sacco, O., Romano, L., Rossi, G. A., & Zegarra-Moran, O. (1998) In Vitro Cell. Dev. Biol. 34, 478-481), and plated onto Costar® Snapwell™ filters that were precoated with NIH3T3-conditioned media. After four days the apical media was removed and the cells were grown at an air liquid interface for >14 days prior to use. This resulted in a monolayer of fully differentiated columnar cells that were ciliated, features that are characteristic of airway epithelia. Non-CF HBE were isolated from non-smokers that did not have any known lung disease. CF-HBE were isolated from patients homozygous for ΔF508-CFTR.

HBE grown on Costar® Snapwell™ cell culture inserts were mounted in an Using chamber (Physiologic Instruments, Inc., San Diego, Calif.), and the transepithelial resistance and short-circuit current in the presence of a basolateral to apical Cl⁻ gradient (I_{SC}) were measured using a voltage-clamp system (Department of Bioengineering, University of Iowa, IA). Briefly, HBE were examined under voltage-clamp recording conditions (V_{hold}=0 mV) at 37° C. The basolateral solution contained (in mM) 145 NaCl, 0.83 K₂HPO₄, 3.3 KH₂PO₄, 1.2 MgCl₂, 1.2 CaCl₂, 10 Glucose, 10 HEPES (pH adjusted to 7.35 with NaOH) and the apical solution contained (in mM) 145 NaGluconate, 1.2 MgCl₂, 1.2 CaCl₂, 10 glucose, 10 HEPES (pH adjusted to 7.35 with NaOH).

### Identification of Potentiator Compounds

Typical protocol utilized a basolateral to apical membrane Cl⁻ concentration gradient. To set up this gradient, normal ringers was used on the basolateral membrane, whereas apical NaCl was replaced by equimolar sodium gluconate (titrated to pH 7.4 with NaOH) to give a large Cl⁻ concentration gradient across the epithelium. Forskolin (10 μM) and all test compounds were added to the apical side of the cell culture inserts. The efficacy of the putative ΔF508-CFTR potentiators was compared to that of the known potentiator, genistein.

### Patch-Clamp Recordings

Total CF current in ΔF508-NIH3T3 cells was monitored using the perforated-patch recording configuration as previously described (Rae, J., Cooper, K., Gates, P., & Watsky, M. (1991) J. Neurosci. Methods 37, 15-26). Voltage-clamp recordings were performed at 22° C. using an Axopatch 200B patch-clamp amplifier (Axon Instruments Inc., Foster City, Calif.). The pipette solution contained (in mM) 150 N-methyl-D-glucamine (NMDG)-Cl, 2 MgCl₂, 2 CaCl₂, 10 EGTA, 10 HEPES, and 240 μg/mL amphotericin-B (pH adjusted to 7.35 with HCl). The extracellular medium contained (in mM) 150 NMDG-Cl, 2 MgCl₂, 2 CaCl₂, 10 HEPES (pH adjusted to 7.35 with HCl). Pulse generation, data acquisition, and analysis were performed using a PC equipped with a Digidata 1320 A/D interface in conjunction with Clampex 8 (Axon Instruments Inc.). To activate ΔF508-CFTR, 10 μM forskolin and 20 μM genistein were added to the bath and the current-voltage relation was monitored every 30 sec.

### Identification of Potentiator Compounds

The ability of ΔF508-CFTR potentiators to increase the macroscopic ΔF508-CFTR CF current (I_{ΔF508}) in NIH3T3 cells stably expressing ΔF508-CFTR was also investigated using perforated-patch-recording techniques. The potentiators identified from the optical assays evoked a dose-dependent increase in IΔ_{F508} with similar potency and efficacy observed in the optical assays. In all cells examined, the reversal potential before and during potentiator application was around −30 mV, which is the calculated E_{Cl} (−28 mV).

### Cell Culture

NIH3T3 mouse fibroblasts stably expressing ΔF508-CFTR are used for whole-cell recordings. The cells are maintained at 37° C. in 5% CO₂ and 90% humidity in Dulbecco's modified Eagle's medium supplemented with 2 mM glutamine, 10% fetal bovine serum, 1×NEAA, β-ME, 1×pen/strep, and 25 mM HEPES in 175 cm² culture flasks. For whole-cell recordings, 2,500-5,000 cells were seeded on poly-L-lysine-coated glass coverslips and cultured for 24-48 hrs at 27° C. before use to test the activity of potentiators; and incubated with or without the correction compound at 37° C. for measuring the activity of correctors.

### Single-Channel Recordings

Gating activity of wt-CFTR and temperature-corrected tF508-CFTR expressed in NIH3T3 cells was observed using excised inside-out membrane patch recordings as previously described (Dalemans, W., Barbry, P., Champigny, G., Jallat, S., Dott, K., Dreyer, D., Crystal, R. G., Pavirani, A., Lecocq, J-P., Lazdunski, M. (1991) Nature 354, 526-528) using an Axopatch 200B patch-clamp amplifier (Axon Instruments Inc.). The pipette contained (in mM): 150 NMDG, 150 aspartic acid, 5 CaCl₂, 2 MgCl₂, and 10 HEPES (pH adjusted to 7.35 with Tris base). The bath contained (in mM): 150 NMDG-Cl, 2 MgCl₂, 5 EGTA, 10 TES, and 14 Tris base (pH adjusted to 7.35 with HCl). After excision, both wt- and ΔF508-CFTR were activated by adding 1 mM Mg-ATP, 75 nM of the catalytic subunit of cAMP-dependent protein kinase (PKA; Promega Corp. Madison, Wis.), and 10 mM NaF to inhibit protein phosphatases, which prevented current rundown. The pipette potential was maintained at 80 mV. Channel activity was analyzed from membrane patches containing ≦2 active channels. The maximum number of simultaneous openings determined the number of active channels during the course of an experiment. To determine the single-channel current amplitude, the data recorded from 120 sec of ΔF508-CFTR activity was filtered “off-line” at 100 Hz and then used to construct all-point amplitude histograms that were fitted with multigaussian functions using Bio-Patch Analysis software (Bio-Logic Comp. France). The total microscopic current and open probability (Pₒ) were determined from 120 sec of channel activity. The Pₒ was determined using the Bio-Patch software or from the relationship Pₒ=I/i(N), where I=mean current, i=single-channel current amplitude, and N=number of active channels in patch.

### Cell Culture

NIH3T3 mouse fibroblasts stably expressing ΔF508-CFTR are used for excised-membrane patch-clamp recordings. The cells are maintained at 37° C. in 5% CO₂ and 90% humidity in Dulbecco's modified Eagle's medium supplemented with 2 mM glutamine, 10% fetal bovine serum, 1×NEAA, β-ME, 1×pen/strep, and 25 mM HEPES in 175 cm² culture flasks. For single channel recordings, 2,500-5,000 cells were seeded on poly-L-lysine-coated glass coverslips and cultured for 24-48 hrs at 27° C. before use.

### Examples: Activity of the Compounds of Formula I

Compounds of Formula I are useful as modulators of ATP binding cassette transporters. Examples of activities and efficacies of the compounds of Formula I are shown below in Table 1-15. The compound activity is illustrated with “+++” if activity was measured to be less than 2.0 μM, “++” if activity was measured to be from 2 μM to 5.0 μM, “+” if activity was measured to be greater than 5.0 μM, and “−” if no data was available. The efficacy is illustrated with “+++” if efficacy was calculated to be greater than 100%, “++” if efficacy was calculated to be from 100% to 25%, “+” if efficacy was calculated to be less than 25%, and “−” if no data was available. It should be noted that 100% efficacy is the maximum response obtained with 4-methyl-2-(5-phenyl-1H-pyrazol-3-yl)phenol.

TABLE 1-15
Activities and Efficacies of the compounds of Formula I

| Example | Activity | |
|---|---|---|
| Compound No. | EC₅₀ (Mm) | % Efficacy |
| 1 | +++ | ++ |
| 1-2 | +++ | ++ |
| 1-3 | +++ | ++ |
| 1-4 | +++ | ++ |
| 1-5 | +++ | +++ |
| 1-6 | +++ | +++ |
| 1-7 | +++ | ++ |
| 1-8 | +++ | ++ |
| 1-9 | +++ | ++ |
| 1-10 | +++ | +++ |
| 1-11 | +++ | ++ |
| 1-12 | +++ | ++ |
| 1-13 | +++ | ++ |
| 1-14 | +++ | ++ |

### IV.B. Protocol 2: Assays for Detecting and Measuring ΔF508-CFTR Correction Properties of Compounds

### Membrane Potential Optical Methods for Assaying ΔF508-CFTR Modulation Properties of Compounds.

The optical membrane potential assay utilized voltage-sensitive FRET sensors described by Gonzalez and Tsien (See Gonzalez, J. E. and R. Y. Tsien (1995) “Voltage sensing by fluorescence resonance energy transfer in single cells” Biophys J 69(4): 1272-80, and Gonzalez, J. E. and R. Y. Tsien (1997) “Improved indicators of cell membrane potential that use fluorescence resonance energy transfer” Chem Biol 4(4): 269-77) in combination with instrumentation for measuring fluorescence changes such as the Voltage/Ion Probe Reader (VIPR) (See, Gonzalez, J. E., K. Oades, et al. (1999) “Cell-based assays and instrumentation for screening ion-channel targets” Drug Discov Today 4(9): 431-439).

These voltage sensitive assays are based on the change in fluorescence resonant energy transfer (FRET) between the membrane-soluble, voltage-sensitive dye, DiSBAC₂(3), and a fluorescent phospholipid, CC2-DMPE, which is attached to the outer leaflet of the plasma membrane and acts as a FRET donor. Changes in membrane potential (Vₘ) cause the negatively charged DiSBAC₂(3) to redistribute across the plasma membrane and the amount of energy transfer from CC2-DMPE changes accordingly. The changes in fluorescence emission were monitored using VIPR II, which is an integrated liquid handler and fluorescent detector designed to conduct cell-based screens in 96- or 384-well microtiter plates.

### Identification of Corrector Compounds

To identify small molecules that correct the trafficking defect associated with ΔF508-CFTR; a single-addition HTS assay format was developed. The cells were incubated in serum-free medium for 16 hrs at 37° C. in the presence or absence (negative control) of test compound. As a positive control, cells plated in 384-well plates were incubated for 16 hrs at 27° C. to “temperature-correct” ΔF508-CFTR. The cells were subsequently rinsed 3× with Krebs Ringers solution and loaded with the voltage-sensitive dyes. To activate ΔF508-CFTR, 10 μM forskolin and the CFTR potentiator, genistein (20 μM), were added along with Cl⁻-free medium to each well. The addition of Cl⁻-free medium promoted Cl⁻ efflux in response to ΔF508-CFTR activation and the resulting membrane depolarization was optically monitored using the FRET-based voltage-sensor dyes.

### Identification of Potentiator Compounds

To identify potentiators of ΔF508-CFTR, a double-addition HTS assay format was developed. During the first addition, a Cl⁻-free medium with or without test compound was added to each well. After 22 sec, a second addition of Cl⁻-free medium containing 2-10 μM forskolin was added to activate ΔF508-CFTR. The extracellular Cl⁻ concentration following both additions was 28 mM, which promoted Cl⁻ efflux in response to ΔF508-CFTR activation and the resulting membrane depolarization was optically monitored using the FRET-based voltage-sensor dyes.

### Solutions

Bath Solution #1: (in mM) NaCl 160, KCl 4.5, CaCl₂ 2, MgCl₂ 1, HEPES 10, pH 7.4 with NaOH.

Chloride-free bath solution: Chloride salts in Bath Solution #1 (above) are substituted with gluconate salts.

CC2-DMPE: Prepared as a 10 mM stock solution in DMSO and stored at −20° C.

DiSBAC₂(3): Prepared as a 10 mM stock in DMSO and stored at −20° C.

### Cell Culture

NIH3T3 mouse fibroblasts stably expressing ΔF508-CFTR are used for optical measurements of membrane potential. The cells are maintained at 37° C. in 5% CO₂ and 90% humidity in Dulbecco's modified Eagle's medium supplemented with 2 mM glutamine, 10% fetal bovine serum, 1×NEAA, β-ME, 1×pen/strep, and 25 mM HEPES in 175 cm² culture flasks. For all optical assays, the cells were seeded at 30,000/well in 384-well matrigel-coated plates and cultured for 2 hrs at 37° C. before culturing at 27° C. for 24 hrs for the potentiator assay. For the correction assays, the cells are cultured at 27° C. or 37° C. with and without compounds for 16-24 hours.

Electrophysiological Assays for Assaying ΔF508-CFTR Modulation Properties of Compounds

### Ussing Chamber Assay

Using chamber experiments were performed on polarized epithelial cells expressing ΔF508-CFTR to further characterize the ΔF508-CFTR modulators identified in the optical assays. FRT^{ΔF508-CFTR} epithelial cells grown on Costar Snapwell cell culture inserts were mounted in an Ussing chamber (Physiologic Instruments, Inc., San Diego, Calif.), and the monolayers were continuously short-circuited using a Voltage-clamp System (Department of Bioengineering, University of Iowa, IA, and, Physiologic Instruments, Inc., San Diego, Calif.). Transepithelial resistance was measured by applying a 2-mV pulse. Under these conditions, the FRT epithelia demonstrated resistances of 4 KΩ/cm² or more. The solutions were maintained at 27° C. and bubbled with air. The electrode offset potential and fluid resistance were corrected using a cell-free insert. Under these conditions, the current reflects the flow of Cl⁻ through ΔF508-CFTR expressed in the apical membrane. The I_{SC} was digitally acquired using an MP100A-CE interface and AcqKnowledge software (ν3.2.6; BIOPAC Systems, Santa Barbara, Calif.).

### Identification of Corrector Compounds

Typical protocol utilized a basolateral to apical membrane Cl⁻ concentration gradient. To set up this gradient, normal ringer was used on the basolateral membrane, whereas apical NaCl was replaced by equimolar sodium gluconate (titrated to pH 7.4 with NaOH) to give a large Cl⁻ concentration gradient across the epithelium. All experiments were performed with intact monolayers. To fully activate ΔF508-CFTR, forskolin (10 μM) and the PDE inhibitor, IBMX (100 μM), were applied followed by the addition of the CFTR potentiator, genistein (50 μM).

As observed in other cell types, incubation at low temperatures of FRT cells stably expressing ΔF508-CFTR increases the functional density of CFTR in the plasma membrane. To determine the activity of corrector compounds, the cells were incubated with 10 μM of the test compound for 24 hours at 37° C. and were subsequently washed 3× prior to recording. The cAMP- and genistein-mediated I_{SC} in compound-treated cells was normalized to the 27° C. and 37° C. controls and expressed as percentage activity. Preincubation of the cells with the corrector compound significantly increased the cAMP- and genistein-mediated I_{SC} compared to the 37° C. controls.

### Identification of Potentiator Compounds

Typical protocol utilized a basolateral to apical membrane Cl⁻ concentration gradient. To set up this gradient, normal ringers was used on the basolateral membrane and was permeabilized with nystatin (360 μg/ml), whereas apical NaCl was replaced by equimolar sodium gluconate (titrated to pH 7.4 with NaOH) to give a large CF concentration gradient across the epithelium. All experiments were performed 30 min after nystatin permeabilization. Forskolin (10 μM) and all test compounds were added to both sides of the cell culture inserts. The efficacy of the putative ΔF508-CFTR potentiators was compared to that of the known potentiator, genistein.

### Solutions

Basolateral solution (in mM): NaCl (135), CaCl₂ (1.2), MgCl₂ (1.2), K₂HPO₄ (2.4), KHPO₄ (0.6), N-2-hydroxyethylpiperazine-N′-2-ethanesulfonic acid (HEPES) (10), and dextrose (10). The solution was titrated to pH 7.4 with NaOH.

Apical solution (in mM): Same as basolateral solution with NaCl replaced with Na Gluconate (135).

### Cell Culture

Fisher rat epithelial (FRT) cells expressing ΔF508-CFTR (FRT^{ΔF508-CFTR}) were used for Ussing chamber experiments for the putative ΔF508-CFTR modulators identified from our optical assays. The cells were cultured on Costar Snapwell cell culture inserts and cultured for five days at 37° C. and 5% CO₂ in Coon's modified Ham's F-12 medium supplemented with 5% fetal calf serum, 100 U/ml penicillin, and 100 μg/ml streptomycin. Prior to use for characterizing the potentiator activity of compounds, the cells were incubated at 27° C. for 16-48 hrs to correct for the ΔF508-CFTR. To determine the activity of corrections compounds, the cells were incubated at 27° C. or 37° C. with and without the compounds for 24 hours.

### Whole-Cell Recordings

The macroscopic ΔF508-CFTR current (I_{ΔF508}) in temperature- and test compound-corrected NIH3T3 cells stably expressing ΔF508-CFTR were monitored using the perforated-patch, whole-cell recording. Briefly, voltage-clamp recordings of L_{ΔF508} were performed at room temperature using an Axopatch 200B patch-clamp amplifier (Axon Instruments Inc., Foster City, Calif.). All recordings were acquired at a sampling frequency of 10 kHz and low-pass filtered at 1 kHz. Pipettes had a resistance of 5-6 MΩ when filled with the intracellular solution. Under these recording conditions, the calculated reversal potential for Cl⁻ (E_{Cl}) at room temperature was −28 mV. All recordings had a seal resistance>20 GΩ and a series resistance<15 MΩ. Pulse generation, data acquisition, and analysis were performed using a PC equipped with a Digidata 1320 A/D interface in conjunction with Clampex 8 (Axon Instruments Inc.). The bath contained <250 μl of saline and was continuously perifused at a rate of 2 ml/min using a gravity-driven perfusion system,

### Identification of Corrector Compounds

To determine the activity of corrector compounds for increasing the density of functional ΔF508-CFTR in the plasma membrane, we used the above-described perforated-patch-recording techniques to measure the current density following 24-hr treatment with the corrector compounds. To fully activate ΔF508-CFTR, 10 μM forskolin and 20 μM genistein were added to the cells. Under our recording conditions, the current density following 24-hr incubation at 27° C. was higher than that observed following 24-hr incubation at 37° C. These results are consistent with the known effects of low-temperature incubation on the density of ΔF508-CFTR in the plasma membrane. To determine the effects of corrector compounds on CFTR current density, the cells were incubated with 10 μM of the test compound for 24 hours at 37° C. and the current density was compared to the 27° C. and 37° C. controls (% activity). Prior to recording, the cells were washed 3× with extracellular recording medium to remove any remaining test compound. Preincubation with 10 μM of corrector compounds significantly increased the cAMP- and genistein-dependent current compared to the 37° C. controls.

### Identification of Potentiator Compounds

The ability of ΔF508-CFTR potentiators to increase the macroscopic ΔF508-CFTR Cl⁻ current (I_{ΔF508}) in NIH3T3 cells stably expressing ΔF508-CFTR was also investigated using perforated-patch-recording techniques. The potentiators identified from the optical assays evoked a dose-dependent increase in I_{ΔF508} with similar potency and efficacy observed in the optical assays. In all cells examined, the reversal potential before and during potentiator application was around −30 mV, which is the calculated E_{Cl} (−28 mV).

### Solutions

Intracellular solution (in mM): Cs-aspartate (90), CsCl (50), MgCl₂ (1), HEPES (10), and 240 μg/ml amphotericin-B (pH adjusted to 7.35 with CsOH).

Extracellular solution (in mM): N-methyl-D-glucamine (NMDG)-Cl (150), MgCl₂ (2), CaCl₂ (2), HEPES (10) (pH adjusted to 7.35 with HCl).

### Cell Culture

NIH3T3 mouse fibroblasts stably expressing ΔF508-CFTR are used for whole-cell recordings. The cells are maintained at 37° C. in 5% CO₂ and 90% humidity in Dulbecco's modified Eagle's medium supplemented with 2 mM glutamine, 10% fetal bovine serum, 1×NEAA, β-ME, 1×pen/strep, and 25 mM HEPES in 175 cm² culture flasks. For whole-cell recordings, 2,500-5,000 cells were seeded on poly-L-lysine-coated glass coverslips and cultured for 24-48 hrs at 27° C. before use to test the activity of potentiators; and incubated with or without the corrector compound at 37° C. for measuring the activity of correctors.

### Single-Channel Recordings

The single-channel activities of temperature-corrected ΔF508-CFTR stably expressed in NIH3T3 cells and activities of potentiator compounds were observed using excised inside-out membrane patch. Briefly, voltage-clamp recordings of single-channel activity were performed at room temperature with an Axopatch 200B patch-clamp amplifier (Axon Instruments Inc.). All recordings were acquired at a sampling frequency of 10 kHz and low-pass filtered at 400 Hz. Patch pipettes were fabricated from Corning Kovar Sealing #7052 glass (World Precision Instruments, Inc., Sarasota, Fla.) and had a resistance of 5-8 MΩ when filled with the extracellular solution. The ΔF508-CFTR was activated after excision, by adding 1 mM Mg-ATP, and 75 nM of the cAMP-dependent protein kinase, catalytic subunit (PKA; Promega Corp. Madison, Wis.). After channel activity stabilized, the patch was perifused using a gravity-driven microperfusion system. The inflow was placed adjacent to the patch, resulting in complete solution exchange within 1-2 sec. To maintain ΔF508-CFTR activity during the rapid perifusion, the nonspecific phosphatase inhibitor F⁻ (10 mM NaF) was added to the bath solution. Under these recording conditions, channel activity remained constant throughout the duration of the patch recording (up to 60 min). Currents produced by positive charge moving from the intra- to extracellular solutions (anions moving in the opposite direction) are shown as positive currents. The pipette potential (Vₚ) was maintained at 80 mV.

Channel activity was analyzed from membrane patches containing ≦2 active channels. The maximum number of simultaneous openings determined the number of active channels during the course of an experiment. To determine the single-channel current amplitude, the data recorded from 120 sec of ΔF508-CFTR activity was filtered “off-line” at 100 Hz and then used to construct all-point amplitude histograms that were fitted with multigaussian functions using Bio-Patch Analysis software (Bio-Logic Comp. France). The total microscopic current and open probability (Pₒ) were determined from 120 sec of channel activity. The Pₒ was determined using the Bio-Patch software or from the relationship Pₒ=I/i(N), where I=mean current, i=single-channel current amplitude, and N=number of active channels in patch.

### Solutions

Extracellular solution (in mM): NMDG (150), aspartic acid (150), CaCl₂ (5), MgCl₂ (2), and HEPES (10) (pH adjusted to 7.35 with Tris base).

Intracellular solution (in mM): NMDG-Cl (150), MgCl₂ (2), EGTA (5), TES (10), and Tris base (14) (pH adjusted to 7.35 with HCl).

### Cell Culture

NIH3T3 mouse fibroblasts stably expressing ΔF508-CFTR are used for excised-membrane patch-clamp recordings. The cells are maintained at 37° C. in 5% CO₂ and 90% humidity in Dulbecco's modified Eagle's medium supplemented with 2 mM glutamine, 10% fetal bovine serum, 1×NEAA, β-ME, 1×pen/strep, and 25 mM HEPES in 175 cm² culture flasks. For single channel recordings, 2,500-5,000 cells were seeded on poly-L-lysine-coated glass coverslips and cultured for 24-48 hrs at 27° C. before use.

Using the procedures described above, the activity, (EC₅₀), of Compound 2 has been measured and is shown in Table 2.

TABLE 2
IC50/EC50 Bins: +++ <= 2.0 < ++ <= 3.0 < +
PercentActivity Bins: + <= 25.0 < ++ <= 100.0 < +++

| | | Binned |
|---|---|---|
| Cmpd. | Binned EC50 | MaxEfficacy |
| Compound | +++ | +++ |
| 2 | | |

Using the procedures described above, the activity, i.e. EC50s, of Compound 3 has been measured and is shown in Table 3.

TABLE 3
IC50/EC50 Bins: +++ <= 2.0 < ++ <= 5.0 < +
PercentActivity Bins: + <= 25.0 < ++ <= 100.0 < +++

| | | Binned |
|---|---|---|
| Cmpd. | Binned EC50 | MaxEfficacy |
| Compound | +++ | +++ |
| 3 | | |

What is claimed is:

1. A pharmaceutical composition comprising:
A Compound of Formula I 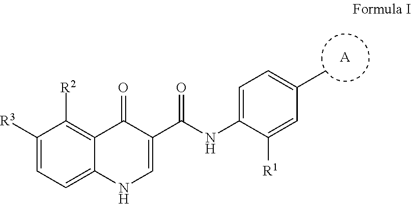
or pharmaceutically acceptable salts thereof, wherein:
ring A is selected from: 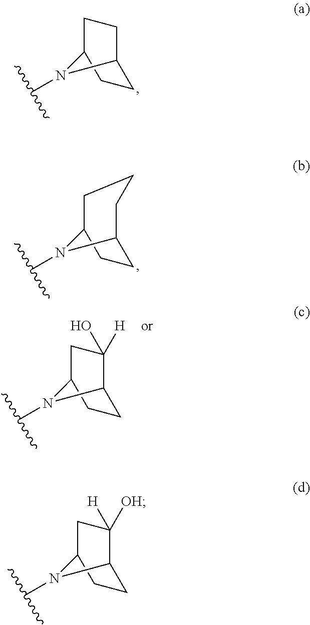
R¹ is —CF₃, —CN, or —C≡CCH₂N(CH₃)₂;
R² is hydrogen, —CH₃, —CF₃, —OH, or —CH₂OH;
R³ is hydrogen, —CH₃, —OCH₃, or —CN;
provided that both R² and R³ are not simultaneously hydrogen; and
one or both of the following:
B. A Compound of Formula II 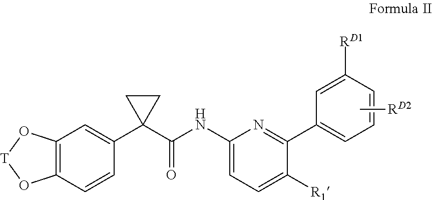
or pharmaceutically acceptable salts thereof, wherein:
T is —CH₂—, —CH₂CH₂—, —CF₂—, —C(CH₃)₂—, or —C(O)—;
R₁′ is H, C₁₋₆ aliphatic, halo, CF₃, CHF₂, O(C₁₋₆ aliphatic); and
R^{D1} or R^{D2} is Z^{D}R₉
wherein:
Z^{D} is a bond, CONH, SO₂NH, SO₂N(C₁₋₆ alkyl), CH₂NHSO₂, CH₂N(CH₃)SO₂, CH₂NHCO, COO, SO₂, or CO; and
R₉ is H, C₁₋₆ aliphatic, or aryl; and/or
C. A Compound of Formula III 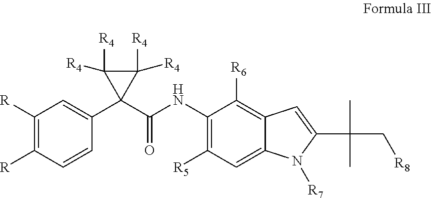
or pharmaceutically acceptable salts thereof, wherein:
R is H, OH, OCH₃ or two R taken together form —OCH₂O— or —OCF₂O—;
R₄ is H or alkyl;
R₅ is H or F;
R₆ is H or CN;
R₇ is H, —CH₂CH(OH)CH₂OH, —CH₂CH₂N⁺(CH₃)₃, or —CH₂CH₂OH;
R₈ is H, OH, —CH₂CH(OH)CH₂OH, —CH₂OH, or R₇ and R₈ taken together form a five membered ring.

2. The pharmaceutical composition of claim 1, comprising a Compound of Formula I and Compound of Formula II.

3. The pharmaceutical composition of claim 1, comprising a Compound of Formula I and Compound of Formula III.

4. The pharmaceutical composition of claim 1, comprising a Compound of Formula I, a Compound of Formula II and a Compound of Formula III.

5. The pharmaceutical composition of claim 1, wherein the Compound of Formula I is Compound 1

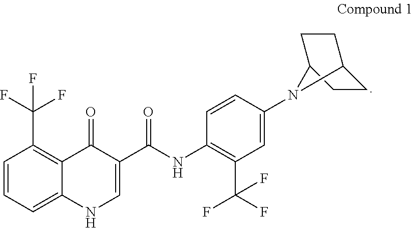

6. The pharmaceutical composition of claim 1, wherein the Compound of Formula II is Compound 2

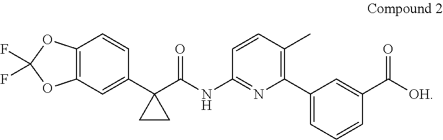

7. The pharmaceutical composition of claim 1, wherein the Compound of Formula III is Compound 3

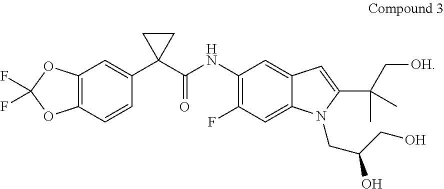

8. The pharmaceutical composition of claim 2, wherein the Compound of Formula I is Compound 1 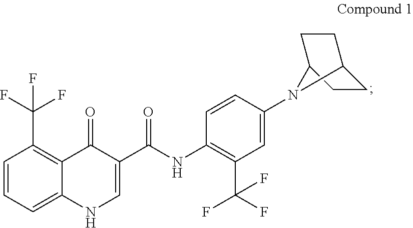
and
the Compound of Formula II is Compound 2

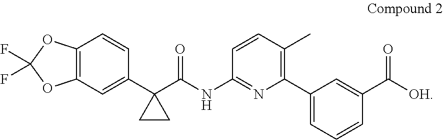

9. The pharmaceutical composition of claim 3, wherein the Compound of Formula I is Compound 1 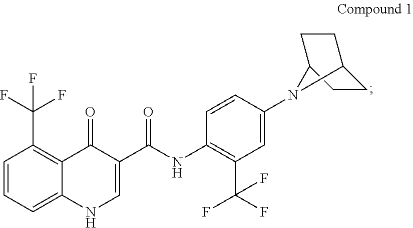
and
the Compound of Formula II is Compound 2

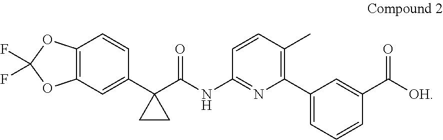

10. The pharmaceutical composition of claim 4, wherein the Compound of Formula I is Compound 1 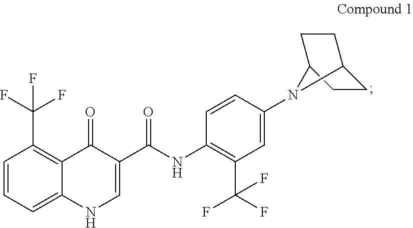
the Compound of Formula II is Compound 2 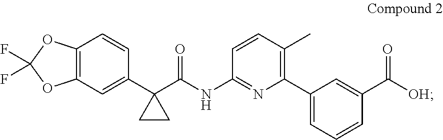
and
the Compound of Formula III is Compound 3

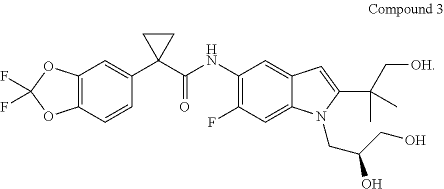

11. A method of treating a CFTR mediated disease in a human comprising administering to the human an effective amount of a pharmaceutical composition according to claim 1.

12. The method of claim 11, wherein the CFTR mediated disease is selected from cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, idiopathic pancreatitis, allergic bronchopulmonary aspergillosis (ABPA), liver disease, hereditary emphysema, hereditary hemochromatosis, coagulation-fibrinolysis deficiencies, such as protein C deficiency, Type 1 hereditary angioedema, lipid processing deficiencies, such as familial hypercholesterolemia, Type 1 chylomicronemia, abetalipoproteinemia, lysosomal storage diseases, such as I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myleoperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases such as Alzheimer's disease, Parkinson's disease, amyotrophic lateral sclerosis, progressive supranuclear palsy, Pick's disease, several polyglutamine neurological disorders such as Huntington's, spinocerebullar ataxia type I, spinal and bulbar muscular atrophy, dentatorubal pallidoluysian, and myotonic dystrophy, as well as spongiform encephalopathies, such as hereditary Creutzfeldt-Jakob disease (due to prion protein processing defect), Fabry disease, Straussler-Scheinker syndrome, COPD, dry-eye disease, or Sjogren's disease, Osteoporosis, Osteopenia, bone healing and bone growth (including bone repair, bone regeneration, reducing bone resorption and increasing bone deposition), Gorham's Syndrome, chloride channelopathies such as myotonia congenita (Thomson and Becker forms), Bartter's syndrome type III, Dent's disease, hyperekplexia, epilepsy, lysosomal storage disease, Angelman syndrome, and Primary Ciliary Dyskinesia (PCD), a term for inherited disorders of the structure and/or function of cilia, including PCD with situs inversus (also known as Kartagener syndrome), PCD without situs inversus and ciliary aplasia.

13. The method of claim 12, wherein the CFTR mediated disease is cystic fibrosis, COPD, emphysema, dry-eye disease or osteoporosis.

14. The method of claim 13, wherein the CFTR mediated disease is cystic fibrosis.

15. The method of claim 14, wherein the patient possesses one or more of the following mutations of human CFTR: ΔF508, R117H, and G551D.

16. The method of claim 15, wherein the method includes treating or lessening the severity of cystic fibrosis in a patient possessing the ΔF508 mutation of human CFTR.

17. The method of claim 15, wherein the method includes treating or lessening the severity of cystic fibrosis in a patient possessing the G551D mutation of human CFTR.

18. The method of claim 16, wherein the method includes treating or lessening the severity of cystic fibrosis in a patient possessing the ΔF508 mutation of human CFTR on at least one allele.

19. The method of claim 16, wherein the method includes treating or lessening the severity of cystic fibrosis in a patient possessing the ΔF508 mutation of human CFTR on both alleles.

20. The method of claim 17, wherein the method includes treating or lessening the severity of cystic fibrosis in a patient possessing the G551D mutation of human CFTR on at least one allele.

21. The method of claim 17, wherein the method includes treating or lessening the severity of cystic fibrosis in a patient possessing the G551D mutation of human CFTR on both alleles.

22. A kit for use in measuring the activity of a CFTR or a fragment thereof in a biological sample in vitro or in vivo, comprising:
a pharmaceutical composition according to claim 1;
(ii) instructions for:
a) contacting the composition with the biological sample;
b) measuring activity of said CFTR or a fragment thereof.

23. The kit of claim 22 further comprising instructions for
a) contacting an additional compound with the biological sample;
b) measuring the activity of said CFTR or a fragment thereof in the presence of said additional compound, and
c) comparing the activity of said CFTR or fragment thereof in the presence of said additional compound with the activity of the CFTR or fragment thereof in the presence of a composition comprising a pharmaceutical composition according to claim 1.

24. The kit of claim 23, wherein the step of comparing the activity of said CFTR or fragment thereof provides a measure of the density of said CFTR or fragment thereof.
